(19) United States
(12) Patent Application Publication
(10) Pub. No.: US 2014/0342369 A1
(43) Pub. Date: Nov. 20, 2014

(54) HAPLOID CELLS

(71) Applicant: IMBA - INSTITUT FÜR MOLEKULARE BIOTECHNOLOGIE GMBH, Vienna (AT)

(72) Inventors: Ulrich Elling, Vienna (AT); Josef Penninger, Vienna (AT); Jasmin Taubenschmid, Stratzing (AT)

(73) Assignee: IMBA - INSTITUT FÜR MOLEKULARE BIOTECHNOLOGIE GMBH, Vienna (AT)

(21) Appl. No.: 14/361,655

(22) PCT Filed: Nov. 30, 2012
(86) PCT No.: PCT/EP2012/074112

§ 371 Date: May 29, 2014

Related U.S. Application Data
(60) Provisional application No. 61/565,626, filed on Dec. 1, 2011.

(30) Foreign Application Priority Data
Nov. 30, 2011 (EP) 11191408.1

(51) Int. Cl. C12Q 1/02; C12Q 1/68

(52) U.S. Cl. CPC C12Q 1/025; C12Q 1/686; C12Q 2600/106
USPC 435/6.12; 435/378; 435/440; 435/325; 435/29; 435/441; 506/10; 435/375

(57) ABSTRACT

The present invention relates to the generation of stable haploid cell cultures, uses of said cells in forward and reverse genetics, especially the identification of target genes associated with a modified phenotype and in particular identifying genetic targets associated with toxin resistance, especially ricin toxicity resistance, and therapeutic uses of target compounds.

The present invention relates to haploid cell and their uses as a genetic screening tool as well as newly identified genes associated with cell survival and toxin resistance.

The diploid genome of complex organisms severely limits many genetic approaches in model species research, but also in human research. Some organisms such as yeast or social insects are haploid, i.e. they carry a single set of chromosomes (Otto and Jarne, 2001). Haploidy in yeast has been utilized to identify fundamental mechanisms of biology (Hartwell et al., 1974). However, all somatic mammalian cells carry two copies of chromosomes, i.e. exhibit diploidy that obscure mutational screens. Organisms with a single copy of their genome, such as yeast, provide a basis for genetic analyses where any recessive mutation of essential genes will show a clear phenotype due to the absence of a second gene copy (Hartwell et al., loc. cit.). It has been shown recently that haploid mammalian cells allow forward genetic screens (Carette et al., 2009, 2011a, 2011b). However, the generation of somatic haploid cells has proved difficult in mammals, likely because haploidy is incompatible with mammalian development (Latham et al., 2002). To this date, haploidy has been achieved in fish embryonic stem cells (Yi et al., 2009), in human KBM-7 leukemia cells (Carette et al., 2009, 2011a), and by electrofusion to generate hybrid cells (Yan et al., 2000). Attempts to establish pluripotent stem cell lines from haploid mouse embryos have resulted in the isolation of parthenogenetic embryonic stem cells with a diploid karyotype (Kaufman et al, 1983). These studies reported the development of apparently normal haploid mouse blastocysts with a defined inner cell mass (Kaufman et al., 1983; Latham et al., 2002). Kaufman et al. (1983) describe a method of activating murine oocytes that develop into a mixture of haploid and diploid stem cells. However the haploid cells could not be stably cultivated and cultures immediately converted into an all diploid state.

The WO 2008/033469 A1 describes methods of producing parthenogenetic embryonic stem cell with a diploid, heterozygous genome. These cells are matched to a certain donor and can be used for transplants.

The WO 01/32015 A1 relates to a method of isolating haploid cells, especially cells that are produced from diploid oocytes (e.g. after fertilization or introduction of a female pronucleus) in which half the chromosomes are then extruded in the polar body. Such haploid cells are converted to diploid cells, e.g. by electrofusion or injection of another haploid genome, to form a diploid cell.

Leeb et al. (2011) describe the generation of a mammalian haploid embryonic stem-like cell culture.

Though loss-of-function genetic screens in model organisms have elucidated numerous biological processes, the diploid genome of mammalian cells has precluded large-scale gene disruption so far. In light of this, means and methods for high-throughput approaches which allow for recessive genetics in mammals would be highly desirable, but are not yet available.

Accordingly, the technical problem underlying the present invention can be seen as the provision of means and methods for complying with the aforementioned needs. The technical problem is solved by the embodiments characterized in the claims and herein below.

Especially, it is a goal of the present invention to provide a culture of haploid stem cells and methods for generating such cells. Such cell should be stable for a sufficient amount of time that allows manipulation and analysis during forward and reverse genetics experiments.

These goals are solved by the invention, which provides a method of generating a mammal cell or cell line of haploid cells comprising obtaining a plurality of haploid cells in an multicellular embryonic stage of development, preferably a blastocyst stage, isolating cells of said plurality, expanding isolated cells of said plurality, selecting and isolating one or more single cells with a haploid genome from said expanded cells, thereby obtaining a cell of a cell line of stable haploid cells. Optionally several isolated cells can be combined to a cell culture. Alternatively, each individual cell can be proliferated and grown into a cell culture (subcloning). The invention further relates to a method for generating a haploid embryonic stem cell line derived from a parthenogenetic morula or blastocyst, the method comprising: (a) activating non-fertilized oocytes from a female subject in vitro to induce parthenogenetic development; (b) culturing said activated oocytes of step (a) to generate morulae and/or blastocysts; (c) isolating embryonic stem cells from said morulae and/or blastocysts of step (c) and, optionally, transferring said embryonic stem cells to a cell culture medium that inhibits differentiation of said embryonic stem cells; (d) subjecting the embryonic stem cells of step (c) to FACS analysis and identifying and/or enriching embryonic stem cells displaying a haploid DNA content; and (e) optionally, repeated FACS purification of the embryonic stem cells displaying a haploid DNA content and expansion of said embryonic stem cells, thereby generating a haploid embryonic stem cell line. Also provided is a cell culture of cells with a haploid genome obtainable or obtained by the inventive method and its preferred variants. The invention further pertains to haploid embryonic stem cell lines generated by the aforementioned method. In addition, the invention provides for morula- or blastocyst-derived embryonic stem-like cell lines comprising at least 60% haploid embryonic stem cells, as well as for kits and test systems using said haploid embryonic stem cells. In addition, the invention relates to methods and uses of haploid stem cells as well as products derived from such methods and uses. Such novel uses and methods are not limited to using the inventively created and improved stem cells, but may also make use of prior haploid stem cells. Further aspects of the invention are described and defined in the claims.

A plurality of haploid cells in a multicellular embryonic stage of development can be obtained from activated oocytes that can be parthenogenically proliferated in a haploid state. In a preferred embodiment the inventive method comprises the steps of obtaining a haploid parthenogenic oozyte or haploid embryonic cell of the mammal, transferring said oozyte or embryonic cell into a pseudopregnant female, allowing said oozyte or embryonic cell to grow to a multicellular stage, optionally culturing cells of said multicellular stage to a blastocyst stage, thereby providing said plurality of haploid cells in an multicellular embryonic stage of development. In further steps the method can comprise obtaining a cell of said multicellular stage, expanding said cells of multicellular stage (simple maintenance e.g. as a culture, especially in isolated form, without expansion is also possible), selecting and isolating a single cell with a haploid genome from said expanded cells, expanding said cell to a cell culture thereby obtaining a cell line of stable haploid cells.

The first step of the method comprises obtaining a haploid parthenogenic oozyte or embryonic cell. This can be done by common methods known in the art, e.g. by activating an oocyte by alcohol or strontium treatment. The activated cells are capable of proliferation and contain only one genome copy (haploidy).

Haploidy relates to the presence of only one genome copy (1n) per cell during the G1 or G0 phase of the cell cycle. The inventive haploid cells are able to replicate the genome in the S phase, establishing a second copy of the genome (2n) in the G2 phase. This copied genome in the G2 phase is homozygous. Haploidy can be distinguished from diploidy, with two genomes (usually heterozygous) during the G1 or G0 phase and four genomes during the G2 phase after the S phase. All inventive cells that eventually lead to the established haploid cell culture maintain haploidy during all steps of the method. Some cell may obtain a diploid genome. These cells are selectively removed.

The activated oocytes can be grown in a pseudopregnant female, wherein said cells are grown to a multicellular stage, such as a blastula or morula stage. Growth in a pseudopregnant female optimizes early embryo-like development of the activated oocyte into haploid cells for culturing. Alternatively, the cells can be cultured as described by Leeb (2011). The cells of the multicellular stage can be isolated and also further grown in vivo or in vitro. In said further growing step the plurality of cells of said multicellular stage can further expand and/or develop, including to a blastula stage. Although haploid cells cannot complete embryonic development while maintaining haploidy, early stages of development are possible. According to the invention such embryo-like multicellular aggregations can be a plurality of haploid cells in an embryonic stage of development. In such a stage the cells are attached by intercellular bonds within said plurality. Such a plurality can be generated de novo or provided, e.g. from a frozen supply. Alternatively it is also possible to simply obtain an isolated cell from such a plurality or multicellular stage, such as a haploid parthenogenic embryo-like stage.

In a next step one or more cell from said plurality of cells in a multicellular stage is isolated and expanded. Isolation of a cell from a multicellular stage usually involves separating the multicellular stage into individual cells, i.e. individualization, e.g. by disrupting cell adhesion such as by trypsinization, or removing at least one haploid cell from the multicellular stage. The isolated cell is then further expanded or maintained, e.g. in a cell culture medium. From the expanded or maintained cells the inventive haploid cell is selected and isolated. It appears that this additional isolation step is crucial when attempting to obtain a cell culture of haploid cells. Although such a cell is most likely present from the beginning of the parthenogenic activation, the presence of other cells obtained from the blastula stage seem to mask the unique properties of a few selected cells that are present in the plurality of cells in a multicellular stage. Only with this additional isolation step does it seem possible that these selected few cells can maintain their haploid state for a prolonged time. Individualization of the multicellular stage, e.g. morula or blastocyst, may be performed on the (entire) multicellular stage or on parts thereof, preferably on the multicellular stage itself. Although it is possible to use only the inner cell mass, e.g. after removing the zona pellucida, removing the trophectoderm and after immunosurgery to obtain and culture the inner cell mass (Leeb, 2011), these steps are not required according to the invention. The invention provides a much simpler method of individualization of the multicellular stage or by culturing cells including the trophectoderm. This method is much easier to handle, is less destructive on the cells of the multicellular stage and saves costs of reagents and labour. In other embodiments, it is of course possible to perform these steps. The zona pellucida of the multicellular stage may or may not be removed during or prior to isolating the cell of said multicellular stage. Inner cell mass may or may not be isolated.

The isolated cells are single cells without attachment to any other cell of said original plurality of cells of multicellular (embryonic) stage. Several isolated cells are usually present in a cell culture. Alternatively, each individual cell can be proliferated and grown into a cell culture (subcloning). Subcloning is particularly advantageous as it provides a standardized origin for further developmental experiments, such as forward mutagenesis experiments and differentiation experiments. If mixtures of cells, potentially deriving from separate original haploid cells are used, is not possible to identify characteristics of individual cells, e.g. the stem-cell like nature or the differentiation capability. In a mixture, one cell might be responsible for expressing stem-cell markers, while another one is responsible to develop into differentiated tissues. According to the invention, it was shown for the first time that single cells are stem cell-like and simultaneously are capable to develop into differentiated cells.

The cells can be maintained and/or expanded on common feeder cultures, preferably on a layer of feeder cells, such as fibroblasts, preferably mouse embryonic fibroblasts (MEF), especially with inactivated cell cycle e.g. by radiation or mitomycin C. The cells may also be maintained or grown in feeder cell free culture conditions. Preferably the cells are maintained on a feeder layer and then maintained in a feeder cell free culture. During the maintenance of the cells, the cells may also be expanded, proliferated and/or grown. Although, immediately after generation of the inventive cells, maintaining the cells on a feeder layer appears important to improve cell viability, the cells could surprisingly adapt for growth in feeder free conditions. Preferably the cells are trained to grow under feeder free conditions by gradually reducing and eventually removing the feeder cell density. Feeder free cells have a significant advantage over feeder dependent cells as e.g. described by Leeb (2011). In a toxicity (resistance) screen when haploid cells are challenged with a toxin and screened for resistances (e.g. after random mutation), the feeder layer would also be affected by the toxin. When feeder cells die, so would the feeder dependent haploid cells—even if a toxicity resistance did develop. Thus false negative results would be caused in feeder cell cultures. Thus preferably, for further uses as described in more detail below, especially for cells being contacted with a toxin or growth inhibitor, a feeder free haploid cell is used.

“Isolation” of a cell as used herein refers to the removal of said cell from other cells of other properties. These other properties may not lie in the genetic make-up of the cells but also in state of activation, differentiation, or expression that may be linked with gene silencing or activation events. In most cases isolation relates to the individualization of single cells but may also relate to isolation of same property or activation status.

Suitable media for stem cell maintenance are known in the art and commercially available; examples are media containing Dulbecco's phosphate buffered saline, MgCl₂, CaCl₂, L glutamine, non-essential amino acids, antibiotics like penicillin/streptomycin (P/S), Fetal bovine serum (FBS), LIF (Leukemia Inhibitory Factor). 0.25% (w/v) trypsin-EDTA may optionally additionally be used to split cells. Additional factors contained in the medium may be human transferrin, putrescine dihydrochloride, human recombinant insulin, L thyroxine, tri-iodothyronine, progesterone, sodium selenite, heparin, and corticosterone.

The initial expansion, after isolation of the cells from a multicellular stage, may be between 10 min to 1 week, preferably, at least 15 min, 20 min or 25 min, and/or up to any one of 5 days, 4 days, 3 days 2 days, 1 days, 18 hours, 12 hours, 6 hours, 3 hours, 1 hour. From the expanded cells single haploid cells are isolated, that can form the basis of a new relatively stable haploid cell culture. These haploid cells have stem cell like characteristics and are referred therein as “stem cells”, although of course the development of a haploid mammal or differentiated tissues of haploid cells is not possible. A “stem cell” is a cell that has the ability to proliferate in culture, producing some daughter cells that remain relatively undifferentiated, and other daughter cells that give rise to cells of one or more specialized cell types. The inventive haploid cells are capable of proliferating indefinitely and can differentiate into the three embryonic germ layers. They cannot differentiate into all somatic cell lineages, and the germ line as embryonic stem cells can. However it is possible to transform the haploid cells into diploid cells which are capable of such diverse differentiation capabilities. In general, the inventive cells may be pluripotent. They are not cells of an embryo since no fertilization of the oocyte occurred. The cells are sometimes referred to embryonic stem or embryonic stem-like since the early stages of development to, e.g. a blastula or morula stage resemble the development of an embryo. However, the inventive cells are as such and without further modification not capable of developing into an embryo or further into a mammal. They are however suitable for development into a cell line. Likewise, “parthenogenic embryos”are for the lack of fertilization not truly embryos.

If all these steps are followed, the inventive method of generating mammal cells or a cell line of haploid cells comprise the steps of obtaining a haploid parthenogenic oozyte or embryonic cell of said mammal, transferring said oozyte or embryonic cell into a pseudopregnant female, allowing said oozyte or embryonic cell to grow to a multicellular stage, preferably morula or blastula stage, optionally culturing cell of said multicellular stage to a blastocyst stage, obtaining a cell of said multicellular stage, expanding said cell of multicellular stage, selecting and isolating one or more single cells with a haploid genome from said expanded cells, thereby obtaining a cell of a cell line of stably haploid cells.

For selection and isolation haploid cells may be labelled, in particular it is preferred to label the genome, chromosomes or DNA in the cells. Preferably haploid cells are fluorescence labelled. To select and isolate single cells may be separated or individualized, e.g. by trypsinization. Individual cells may then be sorted according to their genome content. In special cases, with the method in accordance with the invention the cells are selected and isolated with a high throughput of at least 20, preferably at least 50, more particularly at least 100 and especially preferably 200, cells per second. High throughput methods have the advantage that a large number of cells can be isolated per unit of time. One such method is, for example, flow cytometry or FACS, with which up to 1000 cells per second or more can be categorized and isolated. It is also possible to label the cells according to another marker, e.g. a marker of an embryonic cell, to further distinguish and select the cells. Preferably this additional selection is done concurrently with the selection of haploid cells, such as by using a further fluorescence dye labelling that additional marker (“multicolour”-based method). The cells may further be selected according to size.

As shown herein and in the examples, following these method steps it as possible to generate haploid mouse stem cell lines from parthenogenetic embryos. These cells carry 20 chromosomes, express stem cell markers, and can develop into all germ-layers (endoderm, mesoderm, ectoderm) in vitro and in vivo.

The inventive method comprises culturing an isolated cell or several isolated cells that have been expanded from the multicellular stage or plurality into a cell culture. Especially the method comprises a regular reseeding of the cell culture or enrichment of haploid cells or removal of diploid cells to maintain haploid integrity of the cell culture. Preferably the method further comprises the steps of culturing said cells of the cell culture and isolating one or more haploid cells from said cell culture after up to 50 passages and expanding said isolated haploid cells as new continued cell culture.

The invention provides the first relatively stable culture of a mammal cell. Preferably the parental and/or the cells of the cell culture are from a mammal that is preferably non-human. Thus especially preferred, for ethical reasons, the cell is a non-human cell. The cell may be murine, primate, porcine, bovine, caprine, equine, ovine, canine, feline, rabbit.

The parental cell can be heterozygous or homozygous, preferably heterozygous. A heterozygous cell contains two different genomes that can give rise to an individual haploid genome in the inventive cell line. Each generation event of a haploid cell from a heterozygous cell usually gives rise to a different combination of the parental genomes and thus a different haploid cell. Preferably the inventive cells of the cell culture are clones, comprising identical haploid genomes. It is also possible that the parental cells are heterozygous, i.e. comprise two identical genomes. All haploid cells from such parental cells are usually identical. The invention is of particular advantage for heterozygous parental cells, since it is possible with the inventive methods to generate homozygous progeny cells via the haploid stem cell route.

The inventive haploid cells may be converted to diploid cells by e.g. via failed cytokinesis and/or endoreplication of the genome or fusion of at least two haploid cells, or by other methods known in the art including electrofusion or injection of another haploid genome (such as in WO 01/32015 A1). Such diploid cells are still homozygous and capable of further embryonic or tissue development—even for stages later than germ layer development, including differentiation, e.g. into tissue cells. Thus, the present invention provides the combination homozygous mutagenesis (as further detailed below) with the differentiation potential of stem cells. If other genomes than that of the haploid cells are artificially introduced, the diploid cells may also be generated heterozygous. Equivalently, the genome of the inventive cells can be introduced into other cells to generate heterozygous cells. These other cells may or may not be haploid, e.g. an oocyte, or diploid. Thus the inventive method may further comprise converting a cell of the inventive culture into a diploid cell and optionally further differentiating said cell, e.g. into a somatic stem cell, or a pluripotent or omnipotent stem cell, such as a interior stomach lining, gastrointestinal tract, lung, muscle, bone, blood, urogenital, epidermal or nervous system cell. In particular the cells may be differentiated to hematopoietic stem cells giving rise to blood cells; bone marrow stromal cells (mesenchymal stem cells) that give rise to e.g. bone cells (osteocytes) and cartilage cells (chondrocytes); multipotent peripheral blood stem cells (PBSCs); adult bone marrow stem cells with the potential to give rise to hepatocytes, cardiomyocytes, neural cells and muscle cells; neural stem cells in the brain giving rise to nerve cells (neurons) as well as non-neuronal cells (astrocytes and oligodendrocytes); epithelial stem cells giving rise to e.g. absorptive cells, goblet cells, Paneth cells, and enteroendocrine cells; skin stem cells (epidermal stem cells giving rise to keratinocytes and follicular stem cells giving rise to the hair follicle); hepatic stem cells giving rise to pancreatic endocrine hormone-producing cells; pancreatic stem and progenitor cells, giving rise to islet cells; stem cells and progenitor cells of the eye (corneal and retinal stem cells); mesoangioblasts (vessel-associated stem cells). By way of example, bone marrow cells and cord blood stem cells are therapeutically useful for blood disorders such as leukemia, multiple myeloma and lymphoma. Stem cells from bone marrow and peripheral blood may be injected either into the coronary arteries or directly into the myocardium for treating severe ischaemic heart disease, transplantable cells including mesenchymal stem cells from bone marrow and CD34+cells from peripheral blood. Therapeutic benefit may be increased vascularization of myocardium, and formation of new myocardial cells.

The inventive cultures of haploid cells are relatively stable but single cell conversions to a diploid state may occur. It has been shown that the inventive cells are stable for 70 passages. In some cases 2-3% of cell may convert to diploid cells per day. Preferably the inventive cell cultures are regularly purified to remove diploid cells or reseeded. In preferred embodiments the inventive cell cultures comprise or consist of at least 30% haploid cells, preferably at least 40%, at least 50%, at least 60%, at least 70%, at least 80% or at least 90%, haploid cells. Newly seeded cultures or frozen cultures may comprise or consist of 100% haploid cells.

In preferred embodiments the rate of conversion to diploid cells is at most 10% per day, preferably at most 8% per day, at most 6% per day, at most 5% per day, at most 4% per day or at most 3% per day, especially when averaged over 5 consecutive days.

A cell culture of the invention usually contains a plurality of haploid stem cells, preferably at least 10, especially preferred at least 50, more preferred at least 100, at least 500, at least 1000, at least 5000, at least 10000 at least 50000 or even at least 100000 cells, of which cells with the above percentages, e.g. at least 30%, are haploid.

The inventive haploid stem cells were genetically analysed and several characteristic genes with high discriminatory value in gene expression have been identified by quantitative PCR. In the inventive cells, preferably gene Nanog and/or Sall4 are upregulated as compared to IB 10/C cells. Preferably the upregulation is at least 1.2 times, preferably at least 1.5 times. For Nanog the upregulation may also be at least 2 times.

The inventive haploid cells are further capable of embryoid body (EB) formation. Such EB cells may express the endodermal marker Gata4. Differentiation was further confirmed using real time PCR. The expression of prototypic ES cell markers Nanog, Rex1, Oct4, Sox2, Klf4, Sall4, or Kfl2 or any combination thereof may be downregulated in EBs while mRNA expression of the lineage commitment markers Hand1 (mesoderm, trophectoderm), Nkx2-5 and Brachyury (both mesoderm), Nestin (neural), Gata4, Gata6, Foxa2 (all early endoderm), Sox17 (endoderm and mesoderm), Cxcr4R (endoderm), Keratin18 (ectoderm) or any combination thereof may be upregulated relative to the parental haploid stem cells without EB differentiation. These differences also indicate that the inventive haploid stem cells are capable of differentiation into several lineages of all three germ-layers.

Preferably the inventive haploid cells express at least one stem cell marker, such as Oct4 and/or Sox2. Preferably the haploid cells of the invention are able to form EBs that contain Gata4⁺endodermal cells and/or Tuj1⁺neurons.

It is one particular object of the present invention to provide a method for generating a pluripotent, haploid embryonic stem cell line derived from a parthenogenetic morula or blastocyst, the method comprising:

(a) activating non-fertilized oocytes from a female subject in vitro to induce parthenogenetic development;
(b) culturing said activated oocytes of step (a) to generate morulae and/or blastocysts;
(c) isolating embryonic stem cells from said morulae and/or blastocysts of step (b) and, optionally, transferring said embryonic stem cells to a cell culture medium that inhibits differentiation of said embryonic stem cells;
(d) subjecting the embryonic stem cells of step (c) to FACS analysis and identifying and/or enriching embryonic stem cells displaying a haploid DNA content; and
(e) optionally, repeated FACS purification of the embryonic stem cells displaying a haploid DNA content and expansion of said embryonic stem cells, thereby generating a haploid embryonic stem cell line.

As used herein, the singular forms “a”, “an” and “the” include both singular and plural reference unless the context clearly dictates otherwise. By way of example, “a cell” refers to one or more than one cell.

The terms “comprising”, “comprises” and “comprised of” as used herein are synonyms with “including”, “includes” or “containing”, “contains”, and are inclusive or open-ended and do not exclude additional, non-recited members, elements or method steps. Evidently, the term “comprising” encompasses the term “consisting of”.

The term “in vitro” as used herein denotes outside, or external to, the animal or human body. The term “in vitro” as used herein should be understood to include “ex vivo”. The term “ex vivo” typically refers to tissues or cells removed from an animal or human body and maintained or propagated outside the body, e.g., in a culture vessel. The term “in vivo” as used herein denotes inside, or internal to, the animal or human body.

The term “parthenogenesis” or “parthenogenetic development” as used herein means a process which entails the development of an embryo directly from an oocyte, without fertilization. As used herein, the mentioned terms refer to the process by which activation of the oocyte, i.e. the female gamete, occurs in the absence of sperm penetration. Parthenogenesis as used herein refers to the development of an early stage embryo comprising trophectoderm and inner cell mass that is obtained by activation of an oocyte, comprising DNA of all female origin. Accordingly, “parthenogenetic morula or blastocyst or embryo” refers to a morula/blastocyst/embryo that only contains all female chromosomal DNA that is derived from female gametes. Such parthenogenetic embryonic stages or embryos can be derived by activation of unfertilized female gametes, e.g., unfertilized, mammalian oocytes, as further explained below.

In a further embodiment, it is also envisaged that parthenogenetic embryos can be derived from androgenesis.

“Gametes” are specialized haploid cells (e.g., spermatozoa and oocytes) produced by meiosis and involved in sexual reproduction.

The term “oocyte” as used herein means a female gametocyte or germ cell involved in reproduction. In other words, it is an immature ovum, or egg cell. An oocyte is produced in the ovary during female gametogenesis. Methods for isolation of oocytes are well known in the art. Essentially, this will comprise isolating oocytes from the ovaries or reproductive tract of a female subject (see for example “Principles and Practice of Fertility Preservation”, Cambridge University Press 2011, edited by J. Donnez and S. S. Kim).

Oocytes used in the context of the present invention may be obtained from a mammalian female subject, preferably selected from a human subject, a non-human primate for example of the genus macaca (e.g. Macaca fascicularis, Macaca mulatta) or the genus callithrix (such as Callithrix jacchus) or the family of the great apes (e.g. gorillas, chimpanzees, and orangutans, however specifically excluding in the context of the present invention humans), a mouse, rat, goat, pig, horse, cow, cat, dog, sheep or camel. The oocyte can be a non-fertilized and immature oocyte or a non-fertilized and mature oocyte. Preferably, the oocyte is a non-fertilized and immature oocyte. Said immature oocytes can be matured in vitro by methods described in the literature (see exemplarily Krotz et al. 2010).

In one embodiment of the method of the invention, unfertilized and immature oocytes are isolated and then matured in vitro, prior to the activation.

As used herein, “activation” of an oocyte refers to a process wherein an unfertilized oocyte is (preferably exogenously) activated such that it undergoes a process typically including separation of the chromatid pairs and extrusion of the second polar body, resulting inter alia in oocytes having a haploid number of chromosomes, each with one chromatid. “Activation” also includes methods whereby a cell containing DNA of all female origin is induced to develop into an embryo that has a discernible inner cell mass and trophectoderm, which is useful for producing pluripotent embryonic stem cells. Embodiments of the invention also include activation of oocytes or blastomere cells such as inner cell mass cells or trophoblast cells that have been transplanted with two male (androgenesis) or two female haploid nuclei (gynogenesis). An “activated oocyte” as used herein further includes an unfertilized and optionally mature(d) oocyte which has been incubated with ethanol or strontium chloride, as known in the art (see for example Kaufmann et al. 1983) and as also described in the appended examples. For instance, oocytes from superovulated female mice or human oocytes can be activated by exposing them to ethanol, preferably in a range of 4% or 8%, more preferably 5% to 7% ethanol (preferably in PBS or saline or other suitable buffers) for a period of time of preferably 3 to 7.5 minutes, more preferably for a period of time of 4 to 6 minutes, most preferred for a period of time of 5 minutes (even more preferred for 5 minutes combined with 5% EtOH as shown in the appended examples), or strontium chloride, preferably 25 mM as outlined exemplarily in Leeb et al. 2011. Appropriate culture conditions for the generation of morulae and blastocysts derived from activated oocytes are described in the art (for example Leeb et al. 2011; or Krotz et al. 2010, to name some).

The term “blastomere” as used herein means a type of cell produced by division of the activated oocyte.

The term “morula” as referred to herein is a structure formed in the early embryogenesis of mammals and means a solid ball of about 16 undifferentiated, spherical cells. As cell division continues in the morula, the blastomeres change their shape and tightly align themselves against each other. This process is known as compaction.

The term “blastocyst” as used herein is an early embryonic stage in the mammalian development after the formation of the morula. It is a specifically mammalian example of a blastula. It possesses an inner cell mass (ICM), or embryoblast, which subsequently forms the embryo, and an outer layer of cells, or trophoblast, which later forms the placenta. The ICM is one source for embryonic stem cells of the present invention, while the trophoblast is another source. The trophoblast surrounds the inner cell mass and a fluid-filled blastocyst cavity known as the blastocoele or the blastocystic cavity. Blastocyst formation begins at about day 5 after fertilization in humans, when the blastocoele opens up in the morula. Accordingly, the term “inner cell mass” refers to the inner portion of an embryo which gives rise to fetal tissues. In the context of the present invention, these cells of the inner cell mass (see the appended examples) but also the trophoblasts, preferably Oct4-induced trophoblasts or OiPS as disclosed for example in Wu et al. (2011) can be used to provide a continuous source of the pluripotent embryonic stem cells of the present invention. It will be understood, that trophoblasts that are used in the context of the present invention (preferably induced trophoblast, whereby induced means an induction of the pluripotence of these cells, which is exemplified by Oct4-induced trophoblasts—OiPS) can be derived from the blastocyst (including human blastocyst) without destroying the blastocyst, i.e. the blastocyst-“embryo” will not be harmed—corresponding methods are well known to the skilled person, for example from the technical field of preimplantation diagnosis (sometimes referred to as “PID”) which is frequently conducted on trophoblasts derived from a mammalian (including human) embryo.

In the present invention, the “inner cell mass” refers to the inner portion of the embryo that results from parthenogenesis.

The term “progenitor cell” refers generally to an unspecialized or relatively less specialized and proliferation-competent cell, which or the progeny of which can give rise to at least one relatively more specialized cell type. By means of example and not limitation, a progenitor cell may give rise to descendents that can differentiate along one or more lineages to produce increasingly relatively more specialized cells, wherein such descendents and/or increasingly relatively more specialized cells made themselves be progenitor cells, or even to produce terminally differentiated cells, i.e. fully specialized cells, which may be post-mitotic. The term also encompasses embryonic stem cells as defined herein.

The term “stem cell” as used herein refers to a progenitor cell capable of self-renewal, i.e., which can proliferate without differentiation, whereby the progeny of a stem cell or at least part thereof substantially retains the unspecialized or relatively less specialized phenotype, the differentiation potential, and the proliferation competence of the mother stem cell. The term encompasses stem cells capable of substantially unlimited self-renewal, i.e., wherein the capacity of the progeny or part thereof for further proliferation is not substantially reduced compared to the mother cell, as well as stem cells which display limited self-renewal, i.e., wherein the capacity of the progeny or part thereof for further proliferation is demonstrably reduced compared to the mother cell. Based on the ability to give rise to diverse cell types, a progenitor or stem cell may be usually described as totipotent, pluripotent, multipotent or unipotent.

A “totipotent” cell can differentiate into any cell type in the body, including the germ line following exposure to stimuli like that normally occurring in development. Accordingly, a totipotent cell may be defined as a cell being capable of growing, i.e. developing, into an entire organism.

It will be understood that the cells that are subject of the present invention (including the further embodiments of the present invention) are preferably not totipotent, but (strictly) pluripotent.

In a particular preferred embodiment, the cells of the present invention (including all further embodiments related thereto), are human cells or non-human primate cells, pluripotent, and derived from a blastocyst (preferably from the trophoblast).

A “pluripotent” stem cell is not able of growing into an entire organism, but is capable of giving rise to cell types originating from all three germ layers, i.e., mesoderm, endoderm, and ectoderm, and may be capable of giving rise to all cell types of an organism. As used herein, “pluripotent cell” refers to a cell derived from an embryo produced by activation of a cell containing DNA of all female or male origin that can be maintained in vitro for prolonged, theoretically indefinite period of time in an undifferentiated state that can give rise to different differentiated tissue types, i.e., ectoderm, mesoderm, and endoderm. This includes, for example, but not limited to, mesenchymal stem cells that can differentiate into bone, cartilage and muscle; hematopoietic stem cells that can differentiate into blood, endothelium, and myocardium; neuronal stem cells that can differentiate into neurons and glia; and so on. In one embodiment, the pluripotent state of said cells is maintained by culturing inner cell mass or cells derived from the inner cell mass or trophoblasts (preferably induced trophoblast which are reprogrammed to become pluripotent) of an embryo produced by parthenogenetic methods under appropriate conditions, e.g. by culturing on a fibroblast feeder layer or another feeder layer or well-known culture media that includes optionally leukemia inhibitory factor (LIF). The pluripotent state of such cultured cells can be confirmed by various methods known in the art, e.g., (a) confirming the expression of markers characteristic of pluripotent cells; (b) production of chimeric animals that contain cells that express the genotype of said pluripotent cells; (c) injection of cells into animals, e.g., SCID mice, with the production of different differentiated cell types in vivo; and (d) observation of the differentiation of said cells (e.g., when cultured in the absence of feeder layer or LIF) into embryoid bodies and other differentiated cell types in vitro. The cells of the present invention are preferably pluripotent.

A “multipotent” cell is capable of giving rise to at least one cell type from each of two or more different organs or tissues of an organism, wherein the said cell types may originate from the same or from different germ layers, but is not capable of giving rise to all cell types of an organism. In contrast, a “unipotent” cell is capable of differentiating to cells of only one cell lineage.

An “embryonic stem (ES) cell or cell line” as used herein means a cell or cell line with the characteristics of the, preferably mammalian, embryonic stem cells isolated from morulae or blastocysts (as reported, e.g., by Martin (1981); and Evans (1981)); i.e., embryonic stem cells are capable of proliferating indefinitely and can differentiate into all of the specialized cell types of an organism, including the three embryonic germ layers, all somatic cell lineages, and preferably also the germ line. The term “cell line” as used herein means in its broadest sense a permanently established cell culture or cell population, e.g. derived from a preferably mammalian morula or blastocyst, which is (at least theoretically) capable of dividing indefinitely in culture, given appropriate cell culture conditions. “Derived from morula or blastocyst” means that the embryonic stem cells are isolated from a morula or blastocyst. For example, the embryonic stem cells can be isolated from the inner cell mass or the trophoblast of the blastocyst. Preferably, the morula or blastocyst is not damaged or destroyed by the isolation of the embryonic stem cells, i.e. the “remaining” embryo is not harmed by that procedure. Embryonic stem cells have high nuclear-to-cytoplasm ratio, prominent nucleoli, are capable of proliferating indefinitely and can be differentiated into most or all of the specialized cell types of an organism, such as the three embryonic germ layers, all somatic cell lineages, and preferably also the germ line. Embryonic stem cells that can differentiate into all of the specialized cell types of an organism and can contribute to the germ line are totipotent. In some cases, embryonic stem cells are obtained that can differentiate into almost all of the specialized cell types of an organism; but not into one or a small number of specific cell types. For example, Thomson et al. (1995) described the isolation of a primate embryonic stem cell that, when transferred into another blastocyst, does not contribute to the germ line. The term “embryonic stem cell line” is intended to include embryonic stem-like cells (ES-like cell) which are cell lines isolated from a mammal inner cell mass or epiblast that has a flattened morphology, prominent nucleoli, is immortal, and is capable of differentiating into all somatic cell lineages, and when transferred into another blastocyst cannot contribute to the germ line. For example, it has been found in a recent study that trophoblast stem cells can be converted into ES-like pluripotent stem cells when overexpressing Oct4 (Wu et al. (2011)). In light of this, Oct4 overexpressing trophoblast cells can also be used for the generation of haploid ES-like cells and cell lines in one embodiment of the means and method of the invention. The term “embryonic stem cell line” is also intended to include “inner cell mass-derived cells” (ICM-derived cells) that are cells directly derived from isolated ICMs or morulae without passaging them to establish a continuous ES or ES-like cell line. Methods for making, culturing and using embryonic stem cells are described, e.g., in U.S. Pat. No. 6,235,970 or U.S. Pat. No. 7,592,175, the contents of which are incorporated herein in their entirety. For example, said cells can be cultured under appropriate conditions, e.g. by culturing them on a fibroblast feeder layer or another feeder layer in the presence of leukemia inhibitory factor (LIF) which inhibits differentiation of the ES cells. The term “embryonic stem cell” as used herein comprises cell lines established from isolated embryonic stem cells, primary embryonic stem cells, or a population or cell line thereof. It encompasses embryonic stem cells in their non-differentiated form or in their differentiated form. It also comprises progenitor of embryonic stem cells, cell lines thereof or cell populations comprising such, undifferentiated or differentiated embryonic stem cells. Optionally, said embryonic stem cells are genetically modified, e.g. mutated, as set forth elsewhere herein. Prototypic embryonic stem cell markers are known in the art and include, for example, Alkaline Phosphatase, Oct4, Sox2, Nanog, Klf4, Rex1, Klf2, cMyc Sall4, or SSEA-1. The non-differentiated, pluripotent or totipotent state of embryonic stem cells can be confirmed by various methods known in the art, e.g., by confirming the expression of markers characteristic of pluripotent/totipotent cells. For example, pluripotent/totipotent ES cells can be characterized by high level expression of Oct4 (a member of the POU transcription factors) and Nanog. A critical amount of Oct4 and Nanog is required to sustain stem-cell pluripotency/totipotency. When ES cells are induced to differentiate, Oct4 and Nanog are downregulated, which has proven to be essential for a proper and divergent developmental program (see, e.g., Cavaleri et al. (2003); Mitsui et al. (2003)).

By “propagating haploid embryonic stem cells or cell line(s)” is meant an embryonic stem cell line of proliferating haploid cells produced artificially outside of the haploid cell's host organism. Typically such haploid cell line will be comprised in an in vitro culture. Alternatively, a haploid cell may be propagated in vivo, e.g. by injection into a pseudopregnant mouse female or into a SKID mouse to produce differentiated cell types.

As used herein, the term “isolated cell” or “isolated (haploid) embryonic stem cell” refers generally to a cell that is not associated with one or more cells or one or more cellular components with which the cell is associated in vivo. For example, an isolated cell may have been removed from its native environment, or may result from propagation, e.g., ex vivo propagation, of a cell that has been removed from its native environment. Preferably, the embryonic stem cell as used herein is an isolated embryonic stem cell. For instance, embryonic stem cells may be isolated from embryos, embryonic tissue or embryonic stages, for example, from the morula or blastula stage (e.g the mammalian blastocyst), preferably from the inner cell mass of the blastocyst. However, a recent study by Wu et al. (2011) suggests that trophoblast cells can be re-programmed to pluripotent ES-like cells when overexpressing Oct4. The haploid embryonic stem and ES-like cell lines generated by the method of the invention can be derived, e.g., from a human, a non-human primate such as macaca (e.g. Macaca fascicularis, Macaca mulatta) or the genus calithrix or great apes (e.g. gorillas, chimpanzees, and orangutans), a mouse, rat, goat, cat, dog, sheep or camel.

The term “cell population” as used herein refers generally to a group of cells. Unless indicated otherwise, the term refers to a cell group consisting of or comprising isolated haploid embryonic stem cells of the invention. It is thereby envisaged that the mentioned cell population may still comprise diploid cells, although these cells are in minority, representing for example not more than 20, 15, 10, 5, 4, 3, 2, or 1% of the total cell count of the respective population. A cell population may consist of cells having a common phenotype or may comprise at least a fraction of cells having a common phenotype. Cells are said to have a common phenotype when they are substantially similar or identical in one or more demonstrable characteristics, including but not limited to morphological appearance, the presence, absence or level of expression of particular cellular components or products, e.g., RNA, proteins, cell-specific markers or other substances, activity of certain biochemical pathways, proliferation capacity and/or kinetics, differentiation potential and/or response to differentiation signals or behavior during in vitro cultivation, e.g., adherence, non-adherence, monolayer growth, proliferation kinetics or the like. Such demonstrable characteristics may therefore define a cell population or a fraction thereof. For example, two haploid ES cell lines of the invention designated HMSc1 and HMSc2 showed an expression profile in transcriptome analysis which closely resembled the one of the diploid ES cell line IB10/C (see FIG. 9).

When a cell population is said herein to be “heterogeneous”, this generally denotes a cell population comprising two or more cells or fractions of cells not having a common phenotype, e.g., a cell population comprising cells of two or more different cell types. By means of example and not limitation, a heterogeneous cell population can be isolated from embryonic tissue or stages such as morulae or blastocysts and may comprise diverse cell types including but not limited to cells of the inner cell mass or the trophoblast.

When a cell population is said herein to be homogenous, it consists of cells having a common phenotype, which cells are also haploid. A cell population said herein to be “substantially homogeneous” comprises a substantial majority of cells having a common phenotype and haploidy. A “substantially homogeneous” cell population may comprise at least 70%, at least 80%, at least 85%, preferably at least 90%, at least 95%, 96%, 97%, 98%, or even at least 99% of cells having a common phenotype and haploidy such as the phenotype specifically referred to, e.g. the phenotype of embryonic stem cells as defined herein or progenitors of embryonic stem cells as defined herein. As used herein, the term “substantially homogeneous” does also encompass a homogeneous population. For instance, a haploid embryonic stem cell line of the invention as described in the following examples may be considered as a substantially homogenous or even homogeneous cell population.

The term “isolating” as used herein means taking out of the embryonic stem cells from the morula or inner cell mass or trophoblast of the blastocyst. For example, human embryos reach the blastocyst stage 4-5 days post fertilization, at which time they consist of about 50-150 cells. Preferably, isolating the embryonic stem cells from the morula or blastocyst according to the invention does not result in the destruction or damage of the blastocyst. For example, a single cell of the inner cell mass of a parthenogenetic blastocyst can be isolated and propagated to a haploid embryonic cell line under appropriate cell culture conditions.

The term “generating” a haploid embryonic stem cell line derived from a parthenogenetic morula or blastocyst in the method of the invention means producing a haploid embryonic stem cell line isolated from a morula or blastocyst (derived from a non-fertilized, activated oocyte).

“FACS” or “FACS analysis” is a well described means of measuring certain physical and chemical characteristics of cells or particles as they pass in a fluid stream by a beam of laser light. The term “flow cytometry” derives from the measurement (meter) of single cells (cyto) as they flow past a series of detectors. Flow Sorting extends flow cytometry by using electrical or mechanical means to divert and collect cells with one or more measured characteristics falling within a range or ranges of values set by the user. The major applications of FCM include the analysis of cell cycle, apoptosis, necrosis, multicolor analysis, cell sorting, functional analysis and stem cell analysis. FACS analysis can be used to identify, sort, purify and enrich embryonic stem cells displaying a haploid DNA content, as described in the following examples. “FACS analysis” as used herein refers in the context of the method of the present invention preferably to the enrichment or purification of haploid cells.

As used herein, “diploid cell” refers to a cell, e.g., an oocyte, blastomer or embryonic stem cell, having a diploid DNA content. A diploid oocyte has 40 chromosomes in mouse, one set (20 chromosomes) originating from each parent. A diploid oocyte has 46 chromosomes in human, one set (23 chromosomes) originating from each parent. As used herein, “diploid DNA” refers to 46 chromosomes in human, one male and one female set, and 40 chromosomes in mice. A “diploid cell” has its chromosomes in homologous pairs, and has two copies (2n) of each autosomal genetic locus. A “zygote” is the diploid cell resulting from the fusion of a male and a female gamete during fertilization.

As used herein, “haploid cell” refers to a cell, e.g., an oocyte, blastomer or embryonic stem cell, having a haploid DNA content, wherein the haploid DNA is of all male or all female origin, preferably female origin. Similarly, a haploid (parthenogenetic) blastomere, morula or blastocyst, or an embryonic stem cell or cell line or cell population of the invention are characterized by a haploid DNA content. As used herein “haploid DNA” refers to 23 chromosomes of all male or all female origin in human. In mouse, haploid DNA refers to 20 chromosomes. By “haploid cell” is meant a cell with a haploid number (1n) of chromosomes. As shown in the following examples, haploid embryonic stem cells according to the invention can be generated by the following exemplary procedure: In mice, oocytes from superovulated female mice can be activated by exposing them to 5% ethanol or 25 mM strontium chloride. After activation, the oocytes are transferred to pseudopregnant recipients. At embryonic day (ED) 3.5, compacted morulae and blastocysts are harvested and cultivated under conditions used to derive embryonic stem cells; see also FIG. 1A.

The term “superovulated mice” as used herein means that superovulated mice can be used as a source for donor oocytes, typically at 3.5 days post-coitus (pc). Said mice can be produced, e.g., by an injection regimen of PMSG and hCG hormones to prepubescent females.

The terms “differentiation”, “differentiating” or “derivatives thereof” as used herein denote the process by which an unspecialized or a relatively less specialized cell becomes relatively more specialized. In the context of cell ontogeny, the adjective “differentiated” is a relative term. Hence, a “differentiated cell” is a cell that has progressed further down a certain developmental pathway than the cell it is being compared with. A differentiated cell may, for example, be a terminally differentiated cell, i.e., a fully specialized cell that takes up specialized functions in various tissues and organs of an organism, and which may but need not be post-mitotic. In another example, a differentiated cell may also be a progenitor cell within a differentiation lineage, which can further proliferate and/or differentiate. Similarly, a cell is “relatively more specialized” if it has progressed further down a certain developmental pathway than the cell it is being compared with, wherein the latter is therefore considered “unspecialized” or “relatively less specialized”. A relatively more specialized cell may differ from the unspecialized or relatively less specialized cell in one or more demonstrable phenotypic characteristics, such as, for example, the presence, absence or level of expression of particular cellular components or products, e.g., RNA, proteins, specific cellular markers or other substances, activity of certain biochemical pathways, morphological appearance, proliferation capacity and/or kinetics, differentiation potential and/or response to differentiation signals, etc., wherein such characteristics signify the progression of the relatively more specialized cell further along the said developmental pathway.

Non-limiting examples of differentiation may include, e.g., the change of a pluripotent stem cell into a given type of multipotent progenitor or stem cell, the change of a multipotent progenitor or stem cell into a given type of unipotent progenitor or stem cell, or the change of a unipotent progenitor or stem cell to more specialized cell types or to terminally specialized cells within a given cell lineage. Differentiation of an unspecialized or less specialized cell to a more specialized cell may proceed through appearance of cells with an intermediate degree of specialization. For example, the isolated haploid embryonic stem cells referred to herein can differentiate into all germ layers, i.e. endoderm, ectoderm and mesoderm, in vitro and in vivo, as shown in the following examples.

By “genetic screening,” “genetic diagnosis,” “genetic analysis” and “genetic testing” is meant the analysis of the haploid embryonic cells or cell lines of the present invention by conventional methods to detect the presence or absence of a specific DNA associated with a phenotype, disease or condition. Such methods include, e.g., in situ hybridization, polymerase chain reaction, nested polymerase chain reaction, fluorometric detection methods, RFLP analysis, VNTR or STR detection methods (which screen for usage in a number of tandem repeat dinucleotide or other short tandem repeat (STR) sequences, single-strand conformational polymorphism (SSCP) analysis, denoting gradient gel electrophoresis (DGGE) and mismatch cleavage analysis i.e., by enzymatic (RNAse A) or chemical (piperidine) means. Such methods are also described in the following examples and well known in the art (Sambrook, Molecular Cloning: A Laboratory Manual, Cold Spring Harbor Laboratory, third edition, 2001).

By “genetic selection” is meant the directed choice of a genotype using genetic testing.

By “genetic modification” or “genetic manipulation” is meant the modification of the genome of a cell, preferably a haploid embryonic stem cell of the invention. This includes insertion, deletion and substitute modifications. Preferably the modification will be effected at a target site in the genome. In a preferred embodiment, the modified haploid ES cell will eventually be used in nuclear transplantation for production of an animal which expresses the modified/manipulated gene.

By “multiplication” is meant increasing the number of cells comprising the desired haploid genome of male or female origin.

The term “nuclear transfer” or “nuclear transplantation” refers to a method of cloning wherein the nucleus from a donor cell is transplanted into an enucleated oocyte. Nuclear transfer techniques or nuclear transplantation techniques are known in the literature (see, e.g., the review by Lorthongpanich et al.(2010) and references cited therein). In the subject application, nuclear transfer or nuclear transplantation or NT are used interchangeably. The present definition also embraces the implantation of one or two selected haploid genomes to produce an embryo.

By “lack a functional gene” is meant either the entire gene is missing from the subjects genome, or the gene is mutated to an extent that it can no longer function (e.g., produce a wild-type protein).

By “genetic defect” is meant a nucleic acid deletion or insertion which corresponds to an alteration in transcription of the gene, translation of the gene's mRNA into a protein, alteration of the half-life of the protein or the gene's mRNA or other change from wild-type expression of the gene. Different forms of a given gene are called “alleles.” The “wild-type alleles” of a gene are those that exist at relatively high frequencies in natural populations and yield wild-type or normal phenotypes. Alleles of a gene that result in abnormal or non-wild-type phenotypes are “mutant alleles.” Zygosity refers to the similarity of alleles for a trait in an organism. If both alleles are the same, the organism is homozygous for the trait. If both alleles are different, the organism is heterozygous for that trait. If one allele is missing, it is hemizygous, and if both alleles are missing, it is nullizygous.

The invention relates to the production and multiplication, by any method, of embryonic stem cells containing a haploid chromosome content, the use of these cells for genetic evaluation, such as forward genetic screens or genetic recessive screens, genetic modification or multiplication of a specific haploid genome, and the use of these cells in producing an embryo with a diploid content of DNA.

In order to generate a haploid embryonic stem cell line derived from a parthenogenetic morula or blastocyst, non-fertilized oocytes have to be isolated from a female subject. The female subject of the present invention is a mammalian female, preferably selected from human, non-human primates such as e.g. macaca (e.g. Macaca fascicularis, Macaca mulatta) or the genus calithrix or great apes (e.g. gorillas, chimpanzees, and orangutans), a mouse, rat, goat, cat, dog, sheep or camel etc, as mentioned hereinbefore. Methods for the isolation of mammalian oocytes are described in the art. The isolated oocytes can be immature or mature oocytes. Preferably, the isolated non-fertilized oocyte is an immature oocyte which can be matured in vitro as known in the literature. In a next step, said oocytes are activated in vitro to induce parthenogenetic development which can be carried out by exposing the oocytes to ethanol or strontium salts. For example, 5% or 7% ethanol or 25 mM strontium chloride can be used to this end. The activated oocytes are then cultured under appropriate cell culture conditions which allow the generation of morulae and/or blastocysts. Said culture conditions are known in the art. Said morulae or blastocysts can then be used as a source for the isolation of embryonic stem cells of the present invention, whereby the cells of the present invention are in a most preferred embodiment pluripotent cells derived from the blastocyst. The isolation of embryonic stem cells is described in the art (e.g. Teratocarcinomas and Embryonic Stem Cells: A Practical Approach” Robertson E. J., ed 1987, Oxford: IRL Press; “Manipulating the mouse embryo” Nagy A. et al., 2003, Cold Spring Harbor Press, Third edition). Optionally, the isolated embryonic stem cells are transferred to a cell culture medium that inhibits differentiation of said embryonic stem cells. Means and methods to achieve this are well known to the skilled person and include for example the addition of LIF into the culture media or buffers. The isolated or cultured embryonic stem cells are in a next step subjected to FACS analysis in order to identify and/or enrich embryonic stem cells displaying a haploid DNA content. In a further optional step, FACS purification of the embryonic stem cells displaying a haploid DNA content and expansion of said embryonic stem cells is repeated one or more times, preferably two, three, four, five or more times, thereby generating a stable haploid embryonic stem cell line. The gist of the present invention resides in one aspect in the finding that oocytes which are activated in vitro to induce parthenogenetic development.

FIG. 1A shows an overview of induction of parthenogenesis and the derivation of haploid ES cell lines of the invention from mice. Mouse oocytes were activated with either 5% ethanol or 25 mM strontium chloride (SrCl₂) and implanted into pseudopregnant females. ES cells were then generated from blastocysts and haploid cells subsequently sorted by FACS. Cultures were routinely resorted until stable haploid cells were derived.

The maturation of human oocytes to blastocysts can be carried out by adapted in vitro fertilization (IVF) protocols known in the art. For example, ovarian hyperstimulation can be carried out in order to retrieve multiple eggs. Thereafter, ultrasoundguided transvaginal oocyte retrieval can be performed to obtain oocytes directly from the ovaries. The isolated oocytes can be matured in vitro for 36 to 48 hours. Appropriate parthenogenetic activation as described herein and cell culture conditions known in the art can be used to generate morulae or blastocysts from which human haploid embryonic stem cells of the invention can be isolated.

The invention further pertains to haploid embryonic stem cell lines, as exemplified by haploid murine embryonic stem cell lines. Parthenogenetic embryos develop from haploid oocytes and, thus, contain only the maternal genome. However, all reported cell lines derived from parthenogenetic embryos carry a diploid set of chromosomes (Kaufman et al., 1983). The present inventors hypothesized that haploid cells might still be present in parthenogenetic early embryos and that haploid ES cells could be derived from such blastocysts. To accomplish this, the inventors activated oocytes from superovulated C57BL/6×129 F1 females by exposure to 5% ethanol. Activated oocytes were then transferred into pseudo-pregnant recipients (see FIG. 1A). At embryonic day (ED) 3.5, compacted morulae and blastocysts were harvested and cultivated under conditions used to derive ES cells. FACS analysis showed that a small number of the parthenogenetically derived cells indeed displayed a reduced DNA content (FIG. 8A). Several rounds of FACS purification of this population and subsequent expansion resulted in two independent cell lines derived from two distinct blastocysts, hereafter termed HMSc1 and HMSc2, with a 1n chromosome set in the G1 phase and a 2n chromosome set in the G2 phase of the cell cycle (FIG. 1B, FIG. 8A). Chromosome spreads showed that both cell lines carry a haploid set of 20 chromosomes (FIG. 1C, FIG. 8B). Of note, both cell lines have now been passaged for more than 50 times without any signs of proliferative crisis. Thus, exploiting activation of meiotic oocytes and parthenogenetic derivation of blastocysts has allowed the inventors to establish mouse cells with a haploid chromosome set.

As appreciated by those skilled in the art, the haploid embryonic stem cells and cell lines of the present invention can be used for a variety of approaches, for instance, for the study of imprinting regulation. Since the present invention provides for haploid embryonic stem cells and cell lines of exclusively maternal origin, it offers a novel tool for analyzing the mechanisms governing allele specific expression or repression.

In addition, said haploid embryonic stem cells and cell lines of the present invention can be used for homozygous mutagenesis. Due to the combination of the haploid chromosome content and the intrinsic tendency to become diploid over time, the haploid embryonic cells and cell lines of the invention are ideally suited for generating homozygous mutations that enable recessive phenotype analysis. To this end, the inventors have successfully used retroviral gene trap mutagenesis as well as transposon facilitated mutagenesis to generate homozygous mutations at large scale. In a similar fashion, radiation or chemical agents can be used for random mutagenesis. The inventors have also successfully performed homologous recombination to introduce any desired mutation at will.

Further, the haploid embryonic stem cells and cell lines of the present invention can be used for the rapid and immediate generation of homozygous targeted mice, thereby circumventing the need for time consuming breeding. This is due to the fact that said cells and cell lines show an intrinsic property of diploidization, most likely due to endoreplication or failed cytokinesis. Such diploid, female embryonic stem cells might be able to contribute significantly to many tissues when, for example, injected into murine blastocysts, thus, leading to homozygous murine tissue, either within a homozygous or a chimeric mouse. Preferably, the haploid embryonic stem cells of the invention contribute to the germ line.

Another approach in which the haploid embryonic stem cells and cell lines of the present invention can be applied is semi-cloning in mammals: Semicloning is defined as the artificial genetic mixing of somatic genomes to give rise to new genetic combinations, as is usually the case for sexual reproduction. To this end, the following two experiments can, for example, be carried out:

1) Oozytes can be artificially be fertilized with haploid embryonic stem cell nuclei, as opposed to sperm. If this approach is successful, this would be the first viable offspring of two females.
2) The female pronucleus of fertilized oozytes could be removed and replaced with a haploid genome of the haploid embryonic stem cells of the invention to allow fusion to the male pronucleus. By doing so, the haploid embryonic stem cells could give rise to heterozygous mice and potential mutations introduced could be taken in vivo and analyzed in detail.

Moreover, the haploid embryonic stem cells and cell lines of the present invention can be used for the generation of a genome wide library of gene knockouts in homozygosity in the context of haploid or diploid embryonic stem cells or primary cells by mutagenesis: Such cells may be used in tissue culture or in vivo to study gene function. In order to generate thousands of mutations and sequence them all, the inventors have, for example, established a protocol to mutate, pick, expand, freeze and analyze thousands of clones in parallel. In short, colonies of ES cells derived from a single, randomly mutated cell were picked, each colony was frozen individually and a fraction of the colony was also expanded to extract DNA. Thereby, thousands of individual cell lines with identified mutations could be generated, each separately frozen, as shown in the following examples.

Finally, the haploid embryonic stem cells and cell lines of the present invention can be used for rapid screens in cell culture: Subsequently to a saturated mutagenesis protocol, the cell pool can be used to identify novel gene functions for multiple developmental or pathological processes. Such novel gene functions will in part be interesting drug targets. The required technology for such screens was published recently (Carette et al., 2009). As a proof of principle, the inventors have successfully conducted a screen for ricin-toxicity and identified multiple new genes involved in ricin toxicity.

A detailed analysis of the haploid embryonic stem cells and cell lines of the invention as well as their application in a variety of genetic and pharmaceutical screens and approaches, such as a reversible mutagenesis protocol which allows saturated genetic recessive screens and results in homozygous alleles or forward genetic screens, is demonstrated in the following examples.

In a preferred embodiment of the method of the invention, said non-fertilized oocytes of step (a) are in vitro matured oocytes.

In another preferred embodiment of the method of the invention, the oocytes of step (a) are activated by exposure of the oocytes to ethanol (preferably 5%) or strontium chloride (SrCl2) (preferably 25 mM).

In a further preferred embodiment of the method of the invention, steps (d) and/or (e) is/are repeated at least 5 times.

In a still further preferred embodiment of the method of the invention, the female subject is a mammalian, preferably a human subject, a non-human primate for example of the genus macaca (e.g. Macaca fascicularis, Macaca mulatta) or the genus callithrix (such as Callithrix jacchus) or the family of the great apes (e.g. gorillas, chimpanzees, and orangutans, however specifically excluding in the context of the present invention humans), a mouse, rat, goat, pig, horse, cow, cat, dog, sheep or a camel.

In a preferred embodiment of the method of the invention, the haploid embryonic stem cells of said cell line are characterized by having a 1n chromosome set in the G1 phase and a 2n chromosome set in the G2 phase of the cell cycle.

In another preferred embodiment of the method of the invention, at least 60%, or preferably 65%, 70%, 75%, 80%, 85%, 90%, 95%, 96%, 97%, 98%, 99% or even 100% of the generated embryonic stem cells of said cell line are haploid.

In a further preferred embodiment of the method of the invention, the haploid embryonic stem cells of said cell line are characterized by expression of one or more of the embryonic stem cell markers selected from Alkaline Phosphatase, Oct4, Sox2, Nanog, Klf4, Rex1, Klf2, cMyc Sall4, and/or SSEA-1.

In a still further preferred embodiment of the method of the invention, the haploid embryonic stem cells of said cell line show stable growth for at least 50, 55, 60, 65, 70, 75 or even more passages.

In an also preferred embodiment of the method of the invention, the haploid embryonic stem cells of said cell line maintain haploidy for at least 7, 8, 9, 10, 11, 12, 13, 14, 15, 20, 25, 30, 35, 40, 50 or even more passages.

In another preferred embodiment of the method of the invention, the method further comprises differentiating the haploid embryonic stem cells of said cell line.

In an additional preferred embodiment of the method of the invention, the method further comprises genetically modifying the haploid embryonic stem cells of said cell line.

In a further embodiment, the method further comprises trans-forming the haploid cells of the invention (preferably cells which have been genetically modified, e.g. by way of a mutation or insertion of a nucleic acid sequence, such as a vector etc.) into diploid cells. These diploid cells are isogenic (including an identical set of sex chromosomes).

The invention further pertains to haploid embryonic stem cell lines generated by the aforementioned method of the invention.

In addition, the invention provides also for morula- or blastocyst-derived embryonic stem-like cell lines comprising at least 60% haploid embryonic stem cells.

The definitions and embodiments set forth with respect to the method of the invention apply mutatis mutandis to the haploid embryonic stem cells and cell lines of the invention.

In addition, the invention pertains to a library obtained from the morula- or blastocyst-derived embryonic stem cells or cell line of the invention. Such a library can be used as a tool for forward and reverse genetics at the genomic scale. For example, the inventors have infected 5×10⁸ cells of a freshly FACS purified haploid culture of one of the embryonic stem cell lines of the invention designated HMSc2-27 with a previously reported retrovirus containing a reversible gene trap (Schnutgen et al., 2008) in order to demonstrate the power of mutagenesis in haploid mouse ES cells. This vector also contains removable Oct-4 binding sites (Schnutgen et al., 2008) which allow for insertions into genes that show minimal or none detectable expression in stem cells. After infection, 7.5×10⁶ independent genomic insertions were generated as estimated from colony formation assays. Embryonic stem cell colonies were then pooled and 10 μg of genomic DNA corresponding to 3 million cells were analyzed to map the viral insertion sites by inverse PCR and deep sequencing. The inventors could unambiguously identify 176, 178 insertions. Considering that this library consists of 40× more (7.5×10⁶) independent integrations, the mutagenesis protocol has, in principle, the power to disrupt most genes. The use of this library as a tool for high throughput reverse genetics is shown in the following examples in more detail. Briefly, said system allowed the generation of a knockout cell line for the microRNA processing enzyme Drosha. In addition, in a forward genetic screen, Gpr107 has been identified as a molecule essential for killing by ricin, a toxin being used as a bioweapon. It will be appreciated by those skilled in the art that mutations introduced into the haploid embryonic stem cells of the invention are homozygous in the diploid status of said embryonic stem cells. Accordingly, said diploid embryonic stem cells can be used for forward and reverse genetic approaches as well (see FIG. 6: the integrations result in homozygous mutations of the respective loci).

Also encompassed by the invention is a kit or a test system comprising the morula- or blastocyst-derived embryonic stem cells or cell line of the invention.

The term “kit” as used herein refers to a collection of means comprising the morula- or blastocyst-derived haploid embryonic stem cells or cell line of the invention and an instruction sheet which are provided in separate or common vials in a ready-to-use manner for carrying out the approaches as set forth herein. A kit may further comprise PCR primers or antibodies which specifically bind to prototypic embryonic stem cell markers, such as Oct4, Nanog, SSEA-1, alkaline phosphatase (AP), Rex1, Sox2, KLf4, Klf2, or Sall4, or other markers referred to herein. Pluripotent ES cells can be characterized by high level expression of Oct4 and Nanog. A critical amount of Oct4 and Nanog expression is required to sustain stem-cell pluripotency. When ES cells are induced to differentiate, Oct4 and Nanog are downregulated. The undifferentiated state of ES cells is often characterized by the expression of the cell surface antigens, SSEA-1, SSEA-3 and SSEA-4. SSEA-1 is expressed on the surface of preimplantation-stage murine embryos at the eight-cell stage and has been found on the surface of teratocarcinoma stem cells but not on their differentiated derivatives. SSEA-3 and -4 are synthesized during oogenesis and are present in the membranes of oocytes, zygotes and early cleavage-stage embryos. Biological roles of these carbohydrate-associated molecules have been suggested in controlling cell surface interactions during development. Undifferentiated primate ES cells, human ES cells and human Embryonic Carcinoma (EC) cells express SSEA-3 and SSEA-4, but not SSEA-1. Undifferentiated mouse ES cells, however, do express SSEA-1 but do not express SSEA-3 or SSEA-4. Alkaline Phosphatase is an enzyme in the blood, intestines, liver, and bone cells and exists as membrane-bound isoforms of glycoproteins sharing a common protein structure but differing in carbohydrate content. These enzymes are most active at alkaline pH—hence the name. Undifferentiated human EC, ES and embryonic germ (EG) cells have been shown to express a very high level of the liver/bone/kidney isozyme of alkaline phosphatase. Expression levels of alkaline phosphatase decrease following stem cell differentiation. In another preferred embodiment, the kit can comprise PCR primers specific for lineage commitment markers such as, for instance, Hand1 (mesoderm and trophectoderm), Nkx2-5 and Brachyury (both mesoderm), Nestin (neural), Gata4, Gata6, Foxa2 (all early endoderm), Sox17 (endoderm and mesoderm), Cxcr4R (endoderm), and Keratin18 (ectoderm). Further markers are described in the following examples. In light of this, the kit of the invention can be used, for example, for studying the differentiation (potential) of haploid embryonic stem cells in vitro and in vivo (e.g. in teratoma assays). In another preferred embodiment, the kit comprises additional means for the administration of the haploid embryonic stem cells as set forth herein, such as syringes and injection needles, which can be used e.g. for the injection of the haploid embryonic stem cells of the invention into blastocysts in order to generate transgenic or knockout mice. The instructions comprise information with respect to the cell culture conditions, cell number to be administered, the route of administration, and the like. In a further preferred embodiment, the kit comprises a library obtained from the morula- or blastocyst-derived embryonic stem cells or cell line of the invention.

The invention also relates to a test system comprising the morula- or blastocyst-derived embryonic stem cells or cell line of the invention. The test system is particularly useful for the development of pharmaceuticals, as shown in the following examples.

It is also preferred in the methods and haploid embryonic stem cells and cell lines of the invention that the blastocyst-derived embryonic stem-cells are derived from the inner cell mass (ICM) of the blastocyst. It is further preferred in the methods and haploid embryonic stem cell lines of the invention that the blastocyst-derived embryonic stem cells are derived from the trophoblast, preferably Oct-4 overexpressing trophoblast.

Embryonic stem cells and trophoblast stem (TS) cells are both derived from early embryos, yet these cells have distinct differentiation properties. Embryonic stem cells can differentiate into all three germ layer cell types, whereas TS cells can only differentiate into placental cells. It has not been determined whether TS cells can be converted into ES-like pluripotent stem (PS) cells. In a recent study by Wu et al. (2011) it has been found that overexpression of a single transcription factor, Oct-4, in TS cells is sufficient to reprogram TS cells into a pluripotent state. These Oct-4-induced PS (OiPS) cells have the epigenetic characteristics of embryonic stem cells, including X chromosome reactivation, elevated H3K27 me3 modifications, and hypomethylation of promoter regions in Oct-4 and Nanog genes. Meanwhile, methylation of promoter region in the Elf5 gene occurred during reprogramming of TS cells. The gene expression profile of OiPS cells was very similar to ESCs. Moreover, OiPS cells can differentiate into the three germ layer cell types in vitro and in vivo. More importantly, chimeric mice with germline transmission could be efficiently produced from OiPS cells. These results demonstrate that one single transcription factor, Oct-4, could reprogram the non-embryonic TS cells into PS cells (Wu et al. (2011)).

The inventive haploid cells open the possibility to combine the power of a haploid genome with pluripotency of stem cells to uncover fundamental biological processes in defined cell types at a genomic scale. The inventive cells can therefore be used in mutation analysis, including forward and reverse genetics.

For example, the invention provides the use of a cell with a haploid genome for mutation analysis, comprising introducing a mutation into a genetic locus of interest and observing modified activity of the cell related to said genetic locus. The cells with a haploid genome is preferably a mammal cell, especially with stem cell like properties as described above (such as capability to differentiate into EBs), in particular a cell as obtained or obtainable by the inventive method. Other haploid cells such as described by Leeb et al. (2011), WO 2001/32015 A1, WO 2012/117254 A1 or Elling et al. (2011) may also be used. Due to the haploid nature of the inventive cell recessive mutations can be investigated with increased efficiency due to the absence of other copies of a single gene in other chromosomes. Thus preferably the inventive cells are used to investigate recessive and/or single copy genes in the inventive haploid cells.

Also for this and further uses of the inventive cells, cells may be selected that are stable in haploid form for at least 10 passages, preferably a cell obtainable or obtained by the method according to the present invention. After e.g. 10 passages there should still be at least 40%, at least 50%, at least 60%, at least 70%, at least 75% of the initial haploid cells, especially with the introduced mutation at the genetic locus of interest. Such a genetic locus of interest may e.g. be Drosha or Rarg.

In particular, the invention allows the introduction of conditional knock-out of essential genes (such as Dosha) or knockins of lethal genes by using suitable conditional mutation vectors, e.g. comprising a reversible gene trap. Conditional knockouts preferably facilitate reversible mutations, which can be reversed to a gene-active state upon stimulation, e.g. as in the double-Flex system (Schnutgen et al. 2008; WO 2006/056615 A1; WO 2006/056617 A1; WO 2002/88353 A2; WO 2001/29208 A1; see also FIG. 15). Thus in preferred embodiments of the invention reversible mutations are introduced into the haploid cells, either by random (forward) or site directed (reverse) mutagenesis. Suitable vectors comprising insertion cassette with a reversible mutations, e.g. knock-outs, can be used. After mutation, the cells may or may not be converted diploid cells. This usually has no effect on the usability of the cells since upon diploidization the mutation will be homozygous. Especially preferred, two or more incompatible inversion cassettes are used, such as the incompatible loxP/lox5171 and FRT/F3 site pairs.

Reversion can be also done once the cells have been differentiated (e.g. as described above) or one “repairs” a locus, differentiates the cells under wild type conditions, and then turns around the insertion again to mutagenize in a (optionally terminally) differentiated cell type.

A reversible mutagenesis protocol was developed that allows saturated genetic recessive screens and results in homozygous alleles. This system allowed to generate the first knock-out cell line for the microRNA processing enzyme Drosha.

Thus the invention further provides a cell of a cell culture of haploid cells of the invention, wherein one or more genes of interest have been inactivated. Preferably the cell is of a cell culture wherein all cells have been similarly modified. Especially provided is a feeder free haploid mammal cell. Feeder freedom, or feeder cell independence is described above.

Further provided is a haploid mammal cell comprising a knock-out of the gene Drosha and/or Rarg. Said cell is preferably of a stem cell-like phenotype as described above and is e.g. capable to differentiate into EBs. Said cell is preferably of a cell culture obtained or obtainable by the inventive methods.

Also provided is a method of modifying expression of a gene of interest, such as by knockout or knockdown, in a haploid cell and observing modified activity of the cell related to said gene of interest.

In a further method of genetic analysis and mutagenesis, the inventive cells can be used in classical forward genetics. Random mutations, especially insertional mutations, can be introduced in the genome of the haploid cells, which result in a different phenotype, which in turn may be analysed in selected cells. The mutations may result in increase or decrease in gene expression. A decrease, including a complete ablation of expression, may occur when a gene is disrupted by the mutation. An increase is possible when an e.g. promoter activity is increased or a repressor becomes disrupted or removed from a gene of interest.

Usability of the inventive haploid cells can be increased by introduction of expression cassettes or expression reporters. Therefore the present invention provides a haploid mammalian cells, preferably as obtained as described above, comprising an expression cassette or expression reporter. Further provided is a method of generating such cells, comprising obtaining a haploid mammalian cell, preferably as obtained as described above, and introducing an expression cassettes or expression reporter. Examples of expression reports include positive or negative selection markers, such as antibiotics resistance genes, HPRT1, diphtheria toxin, bioluminescent proteins. The expression reporters can be driven by a pre-selected promoter that activates at a certain condition of interest. Such a condition may be a stage of development of the cell, especially a degree of differentiation or being a certain cell type. Such promoters may be activated by the occurrence of certain DNA modifications of the genome, preselected DNA portions, in particular transgenes, such as BAC transgenes, or by integration of a certain genetic element of interest into a certain gene locus. Such a genetic element may be a knock-out construct as will be detailed below.

Thus the invention also relates to a method of generating cells comprising a phenotype of interest, comprising randomly mutating a plurality of cells with a haploid genome, preferably mammal cells, especially preferred cells obtainable or obtained by the inventive method, and selecting cells having the phenotype of interest.

Such a phenotype of interest is e.g. cell survival or cell growth, especially when said cells are contacted with a toxin or growth inhibitor. Thus cells with modified genes involved in resistance to said toxin or growth inhibitor can be generated. The genetic mutation can then be analysed and said gene identified.

Such a toxin is e.g. ricin or a ricin-like toxin. The toxin may be a ribosome-inhibiting protein, such as PAP, PAP-S, PAP-II, dodecandrin, ricin or modeccin or toxic, especially enzymatically active, parts thereof, such as enzymatic chains like ricin A chain or modeccin A chain (Ready et al., 1984). In particular preferred the toxin is a AB5 toxin, or the A subunit of an AB5 toxin, which comprises the toxic activity. AB5 toxins are e.g. Campylobacter enterotoxin, cholera toxin, heat-labile enterotoxins (e.g. LT and LT-II), pertussis toxin, shiga toxin, verotoxin, EHEC toxin, pseudomonas exotoxin A. With the inventive methods it was possible to identify gene Gpr107 as a molecule essential for killing by ricin, a toxin being used as bioweapon, by such a forward genetic screen. Based on this proof-of-principle it is now of course also possible to generate resistant cell and identify relevant genes against any other toxin or growth inhibitor or other agents that induce a changed phenotype.

The development of resistances to a potent toxin such as ricin demonstrates that the inventive system of haploid stem cell mutation is capable of screening for and obtaining resistances of virtually any toxin or growth inhibitor, preferably including toxic proteins, toxic small molecules or toxic pathogens.

The inventive introduction of random mutations is preferably non-site specific and can potentially target any site in the genome. Preferred mutation inducers are retroviral or lentiviral infections or the used of nucleotide constructs capable of insertion in the genome, such as transposons. Of course minor site preferences of such insertion constructs or viral mutagens may exist, but in general most genes may potentially be targeted. Usually the inventive mutation is a gene disruption, preventing or reducing gene expression of a functional gene product. Further random mutations can be effected by contacting the cells with mutagenic chemicals or exposure to radiation.

For efficient random mutagenesis, a plurality of cells is used in these experiments, each cell potentially gaining a different mutation. Preferably at least 1000 cells, especially preferred at least 5000, at least 10000, at least 50000, at least 100000, at least 200000, at least 500000 or at least 1000000, cells are used.

Also provided is a cell obtained or obtainable by said method having a resistance to a toxin or growth inhibitor. E.g. there is provided a haploid cell having a resistance to a toxin of interest, preferably ricin or a ribosome inhibitory toxin mentioned above, such as PAP, PAP-S, PAP-II, dodecandrin, ricin or modeccin or toxic (ribosome inhibitory) parts thereof, or a AB5 toxin as mentioned above.

The invention also provides a method of screening a cell for genetic targets having activity in toxin resistance comprising generating cells with a toxin or growth inhibitor resistance further comprising identifying a mutation in said cells having toxin resistance as compared to cells without said random mutation.

When a gene of interest, that needs modification, especially inhibition but also increased expression, has been identified by the inventive random screen it is also possible to modify other cells by modifying expression of the identified gene, e.g. by knock-out or knock-down mutations (for expression decreases) or introduction of expression vectors comprising said gene (for expression increase). Such genes have been identified for ricin resistance, which include genes selected from Table 1 or Table 2, in particular Gpr107, Fut9, Tcf711, Slc35c1, Fgfr2, Galnt2, Mid1, B4galt1, B4galnt3, Plcd3, Ror2, Samd4b, Gcnt2, Ggta1. Especially preferred for this and further aspects of the invention are Gpr107, Fut9 and/or Slc35c1. These genes can be inhibited, e.g. by knock-out or knock-down, to generate cells with increased resistance to ricin or other toxins described above. Such generated cells may be haploid cells, such as described above—that have been used to identify these targets, but may also be other cells, including diploid cells. Cells, including diploid cells may be from the same animal sources as described above for the inventive haploid cells.

Thus the present invention also provides a method of modifying a mammalian haploid cell, preferably as obtained by the inventive method described above, and inhibiting a gene of interest or modifying a genetic locus. Preferably such inhibition is by knock-out mutation, e.g. facilitated by homologous recombination of an inactivating gene construct that prevents expression of the gene of interest, or by modifying the gene of interest by using sequence specific nucleases, or by knock-down, especially using inhibitory nucleic acids for RNAi, including siRNA or shRNA. Genes of interest might or may not have been previously identified by an inventive random mutagenesis screen with a selection for a certain phenotype of interest. Genes of interest may be selected from the genes of Table 1 or Table 2, preferably Gpr107, Fut9, Tcf711, Slc35c1, Fgfr2, Galnt2, Mid1, B4galt1, B4galnt3, Plcd3, Ror2, Samd4b, Gcnt2, Ggta1 or any combination thereof. Also provided is a mammalian haploid cell obtainable by this method, comprising an inhibition of a gene as compared to a non-modified parental mammalian haploid cell.

In particular, the invention provides a modified cell with increased resistance to ricin comprising a reduced expression in any one of the proteins of the genes of Table 1 or 2, preferably Gpr107, Fut9, Tcf711, Slc35c1, Fgfr2, Galnt2, Mid1, B4galt1, B4galnt3, Plcd3, Ror2, Samd4b, Gcnt2, Ggta1, or combinations thereof, as compared to an unmodified cell (e.g. parental cell of the same type without modification). Said modification can be by knock-out or knock-down, especially preferred by administration of an inhibitory nucleic acid, such as one that is or encodes an inhibitory RNS, including siRNA or shRNA. Such a modified call may be a haploid cell, especially a cell as obtained by the present invention or any other cell. Preferably the cell is a mammal cell and/or a non-human cell. In other embodiments the cell may be a human cell. Preferably the cell is an isolated cell and/or in vitro or ex vivo. In other embodiments, the cell may be in vivo, e.g. in a living organism. The cell may be haploid, e.g. as obtained by the inventive method, or diploid. Cells, including diploid cells, may be from the same animal sources as described above for the inventive haploid cells.

As used herein, “ex vivo” cell culture refers to culturing cells outside of the body. Ex vivo cell culture includes cell culture in vitro, e.g., in suspension, or in single- or multi-well plates. Ex vivo culture also includes co-culturing cells with two or more different cell types, and culturing in or on 2- or 3-dimensional supports or matrices, including methods for culturing cells alone or with other cell types to form artificial tissues.

The invention further relates to a method of identifying a therapeutic agent against a toxin, comprising identifying a genetic target as mentioned above and contacting a therapeutic candidate molecule with said genetic target or the gene product of said target, preferably in an isolated cell, and identifying binding events of the candidate with the genetic target or gene product or modified resistance of the cell to the toxin. The toxin is preferably ricin or a ribosome inhibitory toxin mentioned above, such as PAP, PAP-S, PAP-II, dodecandrin, ricin or modeccin or toxic (ribosome inhibitory) parts thereof, or a AB5 toxin as mentioned above, and preferably the genetic target is selected from Table 1 or 2, in particular Gpr107, Fut9, Tcf711, Slc35c1, Fgfr2, Galnt2, Mid1, B4galt1, B4galnt3, Plcd3, Ror2, Samd4b, Gcnt2 or Ggta1. But of course other toxins or growth inhibitors can be selected to identify genetic targets that are involved in resistance. In principle it is possible to test any kind of compound on its activity for being a therapeutic agent against a toxin by challenging a cell culture according to the invention. Once a genetic target (including its gene product, the encoded proteins) has been identified according to the invention, it is possible to directly test compounds for their activity to bind modulate, increase or decrease, especially inactivate these genetic targets, including the encoded proteins. Since genomic sequencing projects are complete for most organisms of interest, the sequence of the target gene is known once the genetic target has been identified. It is then easy to screen for candidate inhibitors that target these genes or the transcribed mRNA. Thus candidate nucleotide inhibitors can easily be screened. Inhibitors of proteins can be obtained by contacting a compound library to identify active compounds that bind the proteins. Toxin resistance activity of binders may then be confirmed by analysing the activity on the proteins or the cells or cell cultures of the invention directly. A preferred class of inhibitors targeting the proteins are antibodies. Antibodies against these target proteins can easily be generated by immunizing model animals, such as mice, rats, other rodents, like hamsters or rabbits, goats, etc. Antibodies as used herein also encompass antibody fragments that bind the target protein, including fragments with a Fv portion, including F(ab′), F(ab) or F(ab)₂ antibodies.

The invention also relates to a method of treating toxin, especially ricin poisoning, in a subject or in a cell or inducing a ricin resistance in a subject or in a cell comprising inhibiting any one of the genes or gene products of the genes of Table 1 or Table 2, preferably Gpr107, Fut9, Tcf711, Slc35c1, Fgfr2, Galnt2, Mid1, B4galt1, B4galnt3, Plcd3, Ror2, Samd4b, Gcnt2 or Ggta1 to said subject. The toxin may be a ricin-like toxin. The toxin may be a ribosome-inhibiting protein, such as PAP, PAP-S, PAP-II, dodecandrin, ricin or modeccin or toxic, especially enzymatically active, parts thereof, such as enzymatic chains like ricin A chain or modeccin A chain (Ready et al., 1984). In particular preferred the toxin is a AB5 toxin, or the A subunit of an AB5 toxin, which comprises the toxic activity. AB5 toxins are e.g. Campylobacter enterotoxin, cholera toxin, heat-labile enterotoxins (e.g. LT and LT-II), pertussis toxin, shiga toxin, verotoxin, EHEC toxin, pseudomonas exotoxin A. Inhibiting can be by administration of an inhibitor. An inhibitor of said genes relates to a compound that decreases the level of expression or activity of said genes, or their respective gene products, including the expressed proteins. Said inhibitor can be an antibody or an inhibitory nucleic acid, especially comprising or encoding a small inhibitory RNA molecule, especially preferred an inhibitory nucleic acid that reduces mRNA expression or content of said genes by at least 20%, especially by at least 50%, in a cell. Nucleotide inhibitors can be selected from antisense molecules, ribozymes or small inhibitory RNA molecules (small interfering RNA (siRNA); in the meaning of the present invention, regulatory RNAs such as “micro RNA” (“miRNA”) and “short hairpin RNA” (“siRNA”) are used interchangeably with the term “siRNA”). Inhibition can be by reducing expression, protein content or activity.

A specific embodiment of nucleotide-type inhibitors employs the application of RNA interference (RNAi). RNAi is the process of sequence-specific post-transcriptional gene silencing initiated by double-stranded RNA that is homologous in sequence to the silenced gene. Small interfering RNA (siRNA) duplexes of 21 to 22 nucleotides are shown to be a powerful tool for inhibiting gene function in mammalian cells. Vector-based systems for stable expression of short interfering RNAs are preferred. These systems are based on a vector, in which a synthetic, gene-specific target sequence encoding the siRNA is expressed under the control of a promoter that is suitable for transcription of small, non-coding RNA. The siRNAs are thus produced from the vector following its introduction into mammalian cells by standard transfection (e.g. electroporation, lipofection) or viral infection protocols (e.g. retroviral infection). A suitable small interfering RNA molecule can be easily selected by known methods in the art. E.g. common is the synthesis of a plurality of about 20-50 different said duplexes of 21 or 22 nucleotides in length and contacting test cells. Cells with the most reduced expression of the target gene are selected. According to the present invention, it is of course also possible to use the phenotype of the haploid cells, e.g. toxin or growth inhibitor resistance, as a marker to select the best inhibitory RNA molecules. Methods for selecting designing siRNAs, including selection of the targeted sequence, preparation of the siRNA duplexes, vector design and delivery are well known in the art, e.g. described in detail in U.S. Pat. No. 7,235,654.

As an alternative to siRNA, antisense oligonucleotides can be used as nucleotide-type inhibitors to interfere with the expression of the target genes, gene products or proteins. Anti-sense oligonucleotides are short stretches of nucleotides that are complementary to a region of the target mRNA and can specifically suppress expression of that particular transcript. The antisense nucleic acid can take the form of RNA expressed from a vector, which has been transfected into the cell or take the form of a DNA or RNA oligonucleotide which can be introduced into cells through a variety of means, e.g. by means of cationic liposomes, cationic porphyrins, fusogenic peptides, and artificial virosomes, or cell permeabilization with streptolysin-O and electroporation. Cationic lipids form stable complexes with oligonucleotides, which exhibit improved cellular uptake, thus resulting in enhanced antisense activity.

In a further embodiment, the invention relates to ribozymes directed against a target gene, gene product or protein. Similarly to antisense oligonucleotides, ribozymes bind to substrate RNA through Watson Crick base pairing, which leads to sequence-specific cleavage of transcripts. Two types of ribozymes, the hammerhead ribozyme and the hairpin ribozyme, have been extensively studied due to their small size and rapid kinetics. Their application has been reviewed in several publications. Ribozymes can be imported into the cell by various means, as described above for antisense oligonucleotides, or they can be expressed from a vector, which offers the advantage of continued intracellular production of these molecules.

Preferably, the nucleotide-type inhibitors are produced from a viral vector, e.g. a retroviral or adenoviral vector, an adeno-associated viral vector or lentiviral vector.

In one embodiment, nucleotide-type inhibitors or constructs encoding the nucleotide-type inhibitors are delivered to cells by transfection, i.e. by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, dendrimers and liposomes. A colloidal system may be a lipid-complexed or liposome-formulated nucleotide-type inhibitor. Formulation of the inhibitor, e.g. with various lipid or liposome materials, may then be effected using known methods and materials and delivered to the recipient cell or mammal.

Particular preferred are small molecule inhibitors of any of the genes of Table 1 or Table 2, preferably of genes involved in fucosylation, such as Fut9 or Slc35c1. The molecular target of these inhibitors may be the gene product, i.e. the encoded protein, or a regulatory protein. Preferred fucosylation inhibitors are disclosed in (Büchsel et al., 1980, Burkart et al., 2000; Lee et al., 2003, Hosoguchi et al., 2010; Rillahan et al., 2012). The inhibitor may also be an inhibitor of fucosyltransferases FucTIII, V, VI, and/or VII. Especially preferred inhibitors comprise fluorinated fucose, such as 2F or 6F fluorinated fucose, especially preferred 2F-Peracetyl-Fucose, 6F-Peracetyl Fucose, GDP-2F-Fucose, GDP-6F-Fucose (Rillahan et al., 2012, Burkart et al., 2000). Further preferred inhibitors are Cytidine-5′-monophospho-5-acetamido-9-azido-3,5,9-trideoxy-β-D-glycero-r-D-galacto-non-2-ulosonic acid, 5-Azidoacetamido-3,5-dideoxy-D-glycero-β-D-galacto-non-2-thiophenyl-1-methyl Ester, 5-Azidoacetamido-3,5-dideoxy-D-glycero-β-D-galacto-non-2-ulosonic-1-methyl Ester, 5-Azidoacetamido-3,5-dideoxy-D-glycero-r,β-D-galacto-non-2-ulosonic Acid, Cytidine-5′-monophospho-5-azidoacetamide-3,5-dideoxy-β-D-glycero-r-D-galacto-non-2-ulosonic Acid, Cytidine-5′-monophospho-5-(2-(4-H8R,9S,13S,14S,17S)-3,17-dihydroxy-13-methyl-7,8,9,11,12,13,14,15,16,17-decahydro-6H-cyclopenta[a]phenanthren-17-yl)-1H-1,2,3-triazol-1-yl)acetamido)-3,5-dideoxy-β-D-glycero-r-D-galacto-non-2-ulosonic Acid; 6-Deoxy-6-(2-(4-((8R,9S,13S,14S,17S)-3,17-dihydroxy-13-methyl-7,8,9,11,12,13,14,15,16,17-decahydro-6H-cyclopenta-[a]phenanthren-17-yl)-1H-1,2,3-triazol-1-yl))-β-L-galactopyranos-1-yl-guanosine 5′-Diphosphate; 6-Deoxy-6-(1-methyl-4-(4-pentylphenyl)-1H-1,2,3-triazol-1-yl)-β-L-galactopyranos-1-yl-guanosine 5′-Diphosphate (Hosoguchi et al., 2010); a GDP-fucose compound such as the compound of formula I: Formula I (Lee et al., 2003):

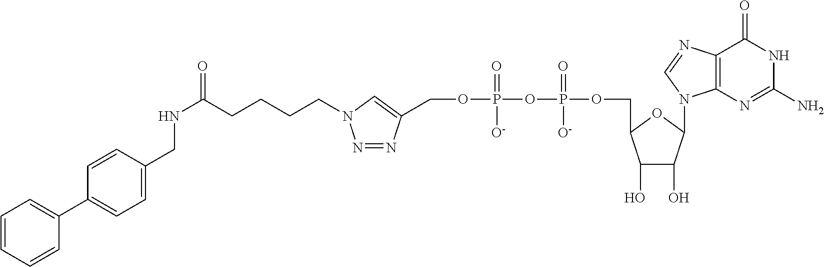 [SMILES: Nc1nc2c(ncn2C2OC(COP(=O)([O-])OP(=O)([O-])OCc3cn(CCCCC(=O)NCc4ccc(-c5ccccc5)cc4)nn3)C(O)C2O)c(=O)[nH]1]

Further preferred inhibitors are selected from Di(trimethylammonium)phosphoryl-2,3,4-tri-O-benz-oyl-6-deoxy-6-uoro-L-fucopyranoside, Di(cyclohexylammonium)phosphoryl 6-fluoro-L-fucopyranoside, Guanosine 5′-diphospho-6-fluono-L-fucopyranoside (Burkart et al., 2000), 2-deoxy-D-galactose (Büchsel et al., 1980).

In particular preferred embodiments, the invention provides the method of treating an AB5 toxin poisoning, preferably ricin or pseudomonas exotoxin A poisoning, comprising administering a fucosylation inhibitor, preferably a small molecule fucosylation inhibitor, especially preferred wherein said inhibitor inhibits a fucosyltransferase, e.g. Fut9, and/or Slc35c1.

Such inhibitors may be administered in form of a pharmaceutical composition. Pharmaceutical compositions may comprise further carrier, including buffers, emulsifiers, and/or additives suitable for a specific mode of administration. The pharmaceutical compositions can be administered systemically or locally, such as, but not limited to, by direct injection into the tissue of interest. The compositions are in general administered intravenously, intramuscularly, as implants, or topically. Suitable pharmaceutical preparation forms are, for example, injectable solutions in ampule form, emulsions, suspensions, creams, aerosols, preparations with sustained release of active compounds.

The present invention is further illustrated by the following figures and examples, without being restricted to these embodiments of the invention.

### FIGURES

FIG. 1. Generation of Haploid Murine Stem Cell Lines.

A) Schematic overview of induction of parthenogenesis and the derivation of haploid stem cell lines. Mouse oocytes were activated with either 5% ethanol (or 25 mM Strontium Chloride (SrCl₂)) and implanted into pseudopregnant females. Stem cells were then generated from blastocysts and haploid cells subsequently sorted by FACS. Cultures were routinely resorted until stable haploid cells were derived. B) Flow cytometric analysis of DNA content in the control diploid stem cell line IB10/C and the haploid HMSc2 cell line. DNA content was determined using Hoechst33342. 1n and 2n chromosome sets for haploid and 2n and 4n chromosome sets for diploid stem cells are indicated. The histograms show data from cells at the 10^{th} sort. C) Representative chromosome spreads of control diploid stem cells and haploid HMSc1 and HMSc2 cells. Spreads from anaphase (1n) and prophase (2n) of mitosis are shown for haploid cells. As a control, anaphase (2n) and prophase (4n) spreads are shown for diploid stem cells. D, E) Sequence coverage relative to the common reference of parental in-house C57BL/6 and 129 strains is shown on a log 2 scale. Haploid cells were derived from C57BL/6×129 crosses. Chromosomes are arranged in numerical order and separated by small gaps. See also FIG. 8.

FIG. 2. Marker Analysis and In Vitro Differentiation Potential of Haploid Stem Cell Lines.

A) Both haploid HMSc1 and HMSc2 cell lines exhibit a morphology characteristic of stem cell colonies (asterisk). Representative phase contrast images are shown. Note the feeder layer of mouse embryonic fibroblasts (MEF) (arrowheads). Haploid cells stain also positive for the stem cell marker alkaline phosphatase (blue, bottom panels). B) Expression of Oct4, Nanog, and Sox2, prototypical markers for murine embryonic stem cells. Phalloidin staining indicates the feeder cell layer. Haploid HMSc1 and HMSc2 cell were co-stained for Oct4 (FITC) and Nanog (TRITC). In both cases, stainings are shown separately in the red channel. Scale bars are 50 μm. Data are from cells after the 4^{th} sort. C) Expression of prototypic stem cell marker genes in the haploid HMSc1 (blue) and HMSc2 (red) cells. mRNA expression was determined using qPCR and normalized to diploid IB10/C ES cells (black bars). D) Gata4 protein expression in embryoid bodies (EB, day 7) as a marker for endoderm. Representative EBs are shown for both haploid HMSc1 and HMSc2 ES cell lines counter-stained with phalloidin (green). Scale bars are 50 μm. E) qPCR revealed down-regulation of the stem cell markers Nanog, Rex1, Oct4, Sox2, Klf2, Klf4, and Sall4 in EBs (day 7) derived from the haploid stem cell line HMSc2 accompanied by expression of the indicated lineage commitment markers (see text). mRNA expression was normalized to the parental, undifferentiated haploid ES cells (set at 1). See also FIG. 9.

FIG. 3. In Vivo Differentiation Potential of Haploid Stem Cell Lines.

A) Haploid stem cells can contribute to tissues in adult mice. Diploid cells from the Agouti⁺clone HMSc2 were injected in into C57BL/6 blastocysts and coat color chimerism was observed (brown fur patches). B) Histological and immunohistological analysis of teratomas derived from control diploid IB10/C stem cells and the haploid ES cell lines HMSc1 and HMSc2. Haploid stem cells can contribute to all three germlayers, namely muscle cells (H&E), intestinal endoderm (mucin producing goblet cells stained by Alcian blue, counterstained with nuclear fast red), Tuj1⁺ neurons, and Cytokeratin 5 (K5) expressing ectoderm. Tuj1⁺ and K5⁺cells were detected by immunohistochemistry (DAB, brown), counter-stained with hematoxylin (blue). Scale bars: 100 μm. See also FIG. 10.

FIG. 4. Haploid Stem Cells have the Intrinsic Ability for Stable Growth and Differentiation.

A) Immunostaining for Oct4 protein expression (red) on three different subclones that were established by plating single haploid cells directly after FACS purification (top panels). The middle and bottom panels show immunostaining for Oct4 (red) and Tuj1 (green) expression and expression of the endodermal marker Gata4 (red, counterstained with DAPI) in attached embryoid bodies (EBs, day 10) derived from the indicated subclones. Data are from cells that were subcloned after >30 passages of the parental line. Scale bars are 50 μm. B) Proliferation rates and C) percentages of haploid cells in control cultures containing 100% diploid MHSc2-27 cells and cultures of HMSc2-27 cells seeded at 80:20 and 50:50 ratios of haploid:diploid cells. Multiplication rates and percent haploidy were determined every 24 hours using FACS analysis of Hoechst33342 stained cells. Note that for this experiment cells were continuously kept in culture for 7 passages (14 days). Based on this experiment, we estimate that-2-3% of haploid cells became diploid each day over the course of the experiment. D) Development of myoblasts from the haploid ES cell subclone HMSc2-27. The feeder cell free diploid stem cell line CCE was used as a control for this experiment. Representative phase contrast images are shown. Scale bars are 100 μm. E) GFP expression (green) in a GFP-tagged haploid stem cell subclone. Non-GFP labeled cells are shown as control (grey shaded histogram). Flow cytometry of DNA content (Hoechst33342) is shown for the same subclone demonstrating that both haploid and diploid cells express GFP. F) Differentiation of haploid and diploid HMSc2-27 cells into EBs (day 13) that contain Tuj1 neurons (green) and Gata4 expressing endodermal cells (red). Note down-regulation of Oct4 expression (red, upper panel) and the presence of residual clusters of Nanog⁺ cells (green, bottom panels). In the top panels, cells were counterstained with DAPI to visualize nuclei. See also FIG. 11-13.

FIG. 5. Differentiation Potential of Haploid Stem Cells.

A) Analysis of the haploid stem cell clone HMSc2-27 cultured under conditions to maintain an stem cell fate (“stem cells”), in vitro differentiated into Nestin⁺neural stem cells (NS cells), and further differentiation into GFAP⁺astrocytes by withdrawal of EGF and FGF2 in the presence of 1% serum (differentiated NS cell culture conditions). Immunofluorescence labeling of Oct4, Nestin, and GFAP are shown, counterstained for DAPI. Representative imagines are shown. Scale bar is 100 μm. B) Flow cytometry analysis of DNA content in cells gated for Oct4 and Nestin expression and grown under stem cell (top), NS cell (middle), and differentiated NS cell (bottom panels) conditions. The gates used and percentages of cells are inserted in each plot. Haploid cells are prominent in Oct4⁺ fractions under all conditions while Nestin⁺ cells differentiated for 4 days are devoid of haploid cells. The red line in the top left panel shows the representative DNA content of the diploid control IB10/C stem cell line gated for Oct4 expression. C) Haploid cells exit the pluripotent state following the same dynamics as diploid cells. The left panel shows control Oct4 levels (mean intensity depicted) in haploid HMSc2-27, mixed (haploid and diploid) HMSc2-27, and control diploid CCE stem cells after 72 h under control (plus LIF) conditions. Differentiation by LIF withdrawal leads to diminished Oct4 expression of diploid and haploid cells (middle panel). Differentiation induction using 0.5 μM retinoic acid results in a rapid loss of Oct4 expression in both haploid and diploid cells, indicative of differentiation (right panel). The same results were obtained when we used 0.1 μM retinoic acid (not shown). Data are shown as mean Oct4 fluorescence intensity +/−SEM analysing more than 50,000 cells per condition. 1way ANOVA (p>0.05) showed increased expression of Oct4 in diploid cells (consistent with increased nuclear area) in all conditions except the 48- and 72-hour retinoic acid treatments wherein Oct4 expression was at background levels. See also FIG. 14.

FIG. 6. Reverse Genetics in Haploid Stem Cells.

A) Analysis of virus integration sites after neomycin selection. 176,178 insertions were determined by deep sequencing. The retrovirus landed in 49% in intergenic and in 51% in intragenic regions, with a high frequency of integration into introns, especially the first intron. B) Graph shows percentage of genes with virus integrations following a single round of retroviral mutagenesis for different fractions of the total viral integration sites (X-axis). Genes with the 10% lowest expression (0-10%) showed the least integration efficiency while higher expressed genes (50-100%) show more efficient gene trapping. For increasing fractions of the total viral integration sites (X-axis) higher saturation is reached, up to 67% yet not reaching saturation, indicating that additional genes are trapped in the total library of 7.5 million independent insertions. C,D) PCR analysis using site specific primers for the indicated genes and a primer specific for the LTR of the inserted retrovirus. The location of the used PCR primers is schematically indicated on top of each panel. Of note, all primers were used for all 10 different genes showing (C) that the virus has indeed integrated into the site identified by initial sequencing and (D) that the integrations result in homozygous mutations of the respective loci. Lane 1=Madcam1; lane 2=Drosha; lane 3=Retinoic acid receptor gamma (Rarg); lane 4=Ap4s1; lane 5=Arap1; lane 6=Evx1; lane 7=Bc1211; lane 8=2210012G02RIK; lane 9=Titin; lane 10=Chr2:50928851. Positive wild type (Wt) and negative H2O controls are shown. E) qPCR analysis of RARG mRNA expression in haploid HMSc2-27 cells that are wild type for rarg (Wt), HMSc2-27 cells that contain the splice acceptor in antisense orientation (AS), and HMSc2-27 cells that contain the splice acceptor in the sense orientation (S). mRNA expression was normalized to the parental HMSc2-27 cells. F) Representative images of cultures containing the indicated wild type, antisense, and sense RARG HMSc2-27 cells treated with 0.1 μM retinoic acid for 10 days. Note the near complete absence of cells in the Wt and antisense cultures. Scale bars are 100 μm. G) qPCR analysis of Drosha mRNA expression in haploid HMSc2-27 cells that are wild type for Drosha (Wt), HMSc2-27 cells that contain the splice acceptor in anti-sense orientation (AS), and HMSc2-27 cells that contain the splice acceptor in the sense orientation (S). mRNA expression was normalized to parental HMSc2-27 cells. H) Complete absence of cystic embryoid bodies in Drosha deficient HMSc2-27 cells as compared to Drosha expressing wild type HMSc2-27 cells and cells containing the splice acceptor in the antisense orientation. Representative images for embryoid bodies are shown on day 10 after EB induction. Of note, a single cystic EB in Drosha mutant cells even in prolonged culture was not observed. Scale bars are 100 μm. I) Histograms showing Venus reporter gene expression in wild type HMSc2-27 cells (Wt), HMSc2-27 cells that contain the splice acceptor in antisense orientation (AS), and HMSc2-27 cells that contain the splice acceptor in the sense orientation (S) transduced with pSENSOR-based miRNA constructs harboring a potent shRNA targeting Firefly Luciferase with (target) or without its target (no target) site in the 3′UTR of Venus. Cells were gated on shRNA expressing (dsRed⁺) cells and Venus expression levels were compared to non-transduced control cells (grey).

FIG. 7. Forward Genetic Screen for Ricin Toxicity in Haploid Stem Cells

A) Haploid HMSc2-27 with/without gene-trap mutagenesis was exposed to ricin from Ricinus communis for 3 weeks. Colonies only appeared in the mutagenized batch and were processed for deep sequencing. B) Top hits identified in the ricin toxicity screen. Sense (green) and antisense (red) insertions in Gpr107, Fut9, and Slc35c1 genomic loci. The vertical lines indicate the respective exons for each gene with the first exon always moved to the left side of each diagram. Insertions in antisense might disrupt gene function, sense integrations will do so in almost all cases. Note that nearly all insertions are in sense for the splice acceptor and that some antisense integrations map to exons, all of which should result in disruptive mutations. Considering that ˜50% of intragenic insertions are sense and ˜50% in antisense, these data also show that the screen has indeed strongly enriched for disruptive mutations (p>1.13E⁻¹⁰ for Gpr107; p>3.95E⁻⁶ for Fut9; p>0.000019 for Slc35c1). C) Genes identified in the ricin toxicity screen. The numbers of distinct retroviral insertions predicted to disrupt gene expression (either because intragenic regions containing the sense orientation of the splice acceptor, or sense and antisense integrations into exons) are indicated. Enrichment for sense mutations vs. anti-sense integrations was assessed using a binomial test and the respective p values are indicated. Of note, anti-sense integrations can also lead to gene disruption. Assigned biochemical pathways and allocation to the Golgi apparatus are also indicated. D, E) Validation of Gpr107 in Ricin toxicity. HMSc2-27 stem cells and NIH3T3 cells were transfected with shRNAs designed to knockdown Gpr107 expression or transfected with control shRNA and then challenged with a lethal dose of ricin for 2 days. Images show representative cultures after 48 hours of ricin treatment (D). Scale bars are 100 μm. (E) The ricin survival rate as ratio between recovered cells of ricin treated vs. ricin untreated cells is shown in % (as determined by quantitative FACS analysis of cells gated for viability by forward scatter, side scatter, and PI staining after 48 hours of ricin treatment). Cells were cultured in 10 cm dishes in triplicates and average survival +/−SD was determined for eGFP negative (untransfected) and eGFP positive haploid HMSc2-27 ES cells and NIH 3T3 cells for each plate.

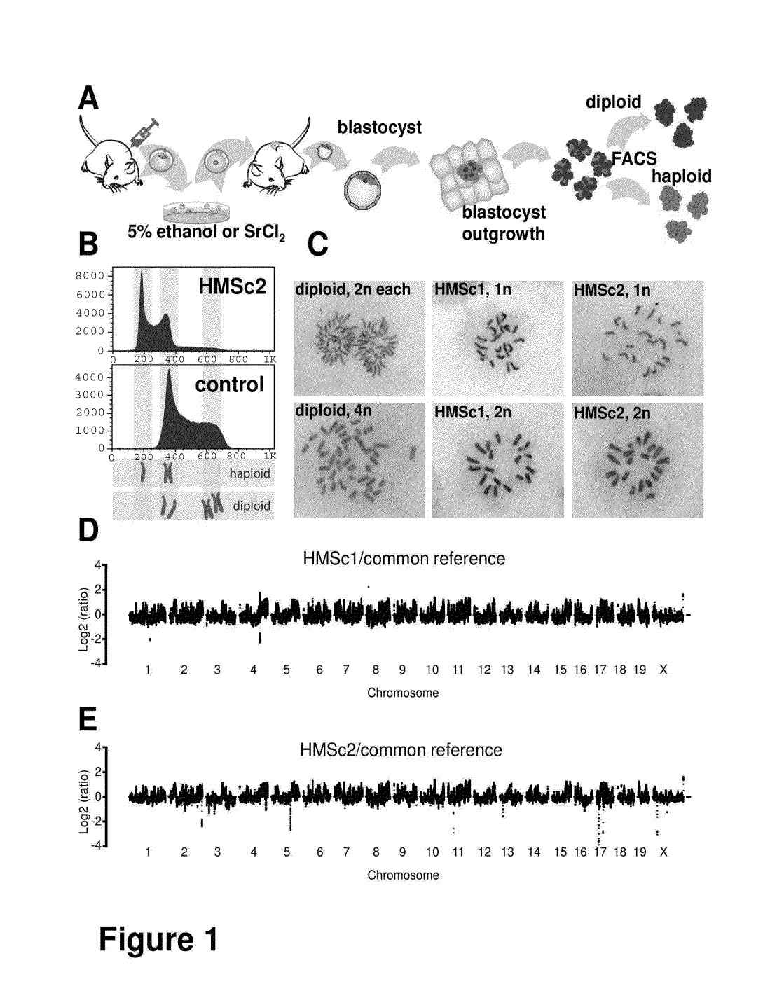

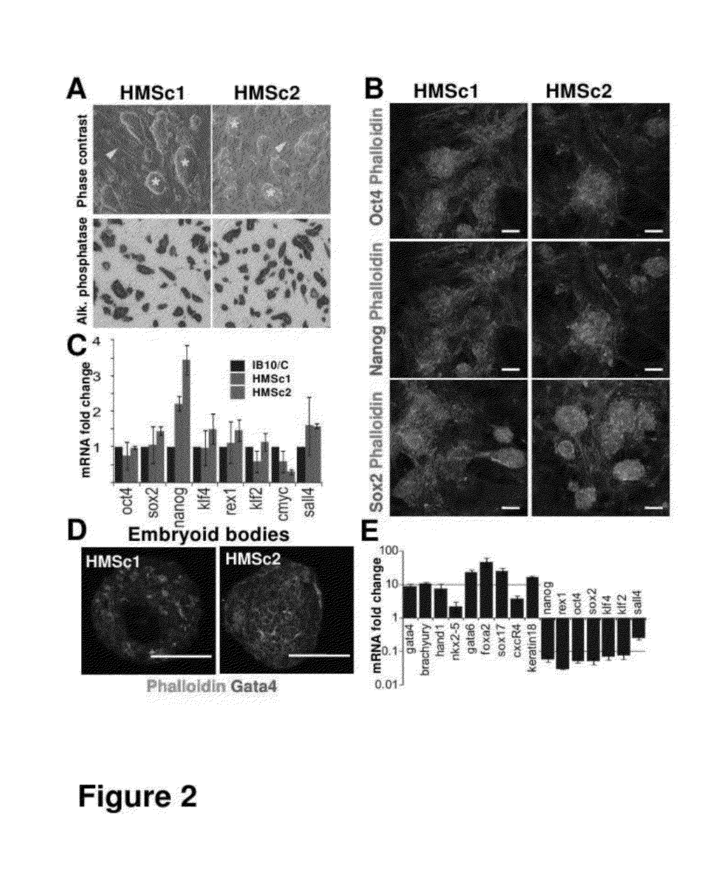

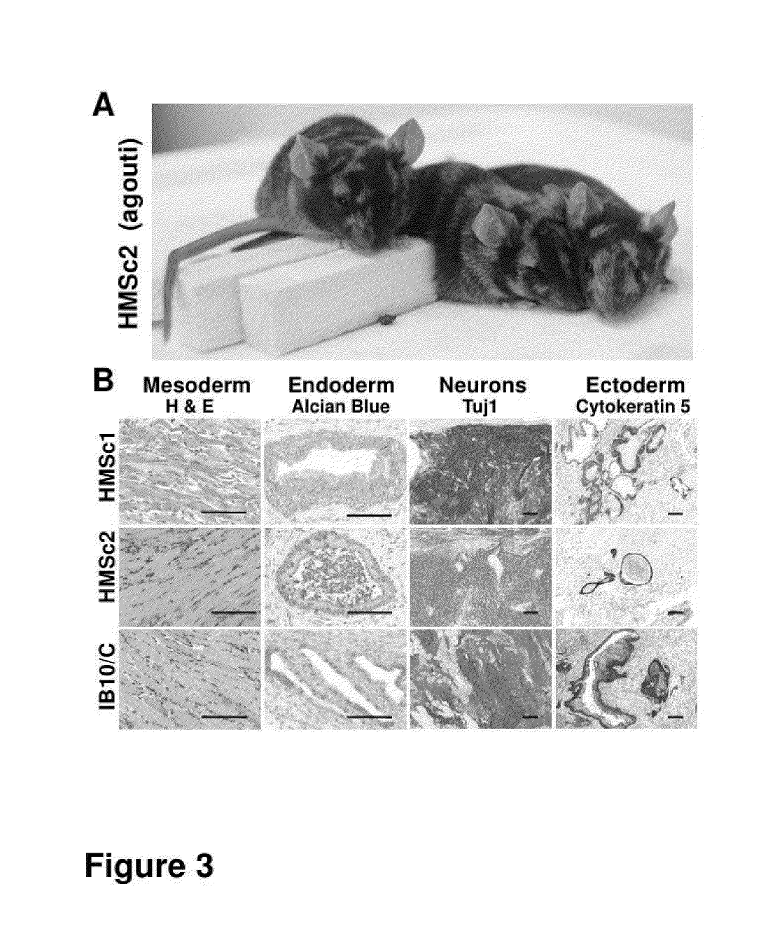

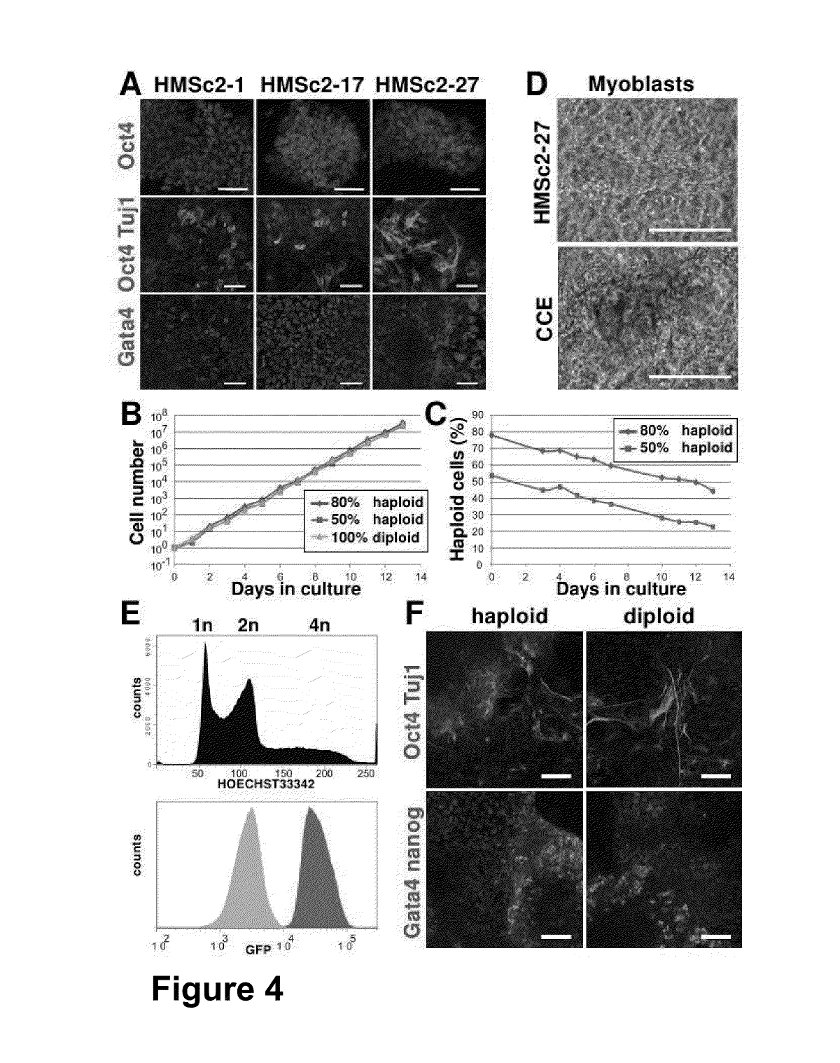

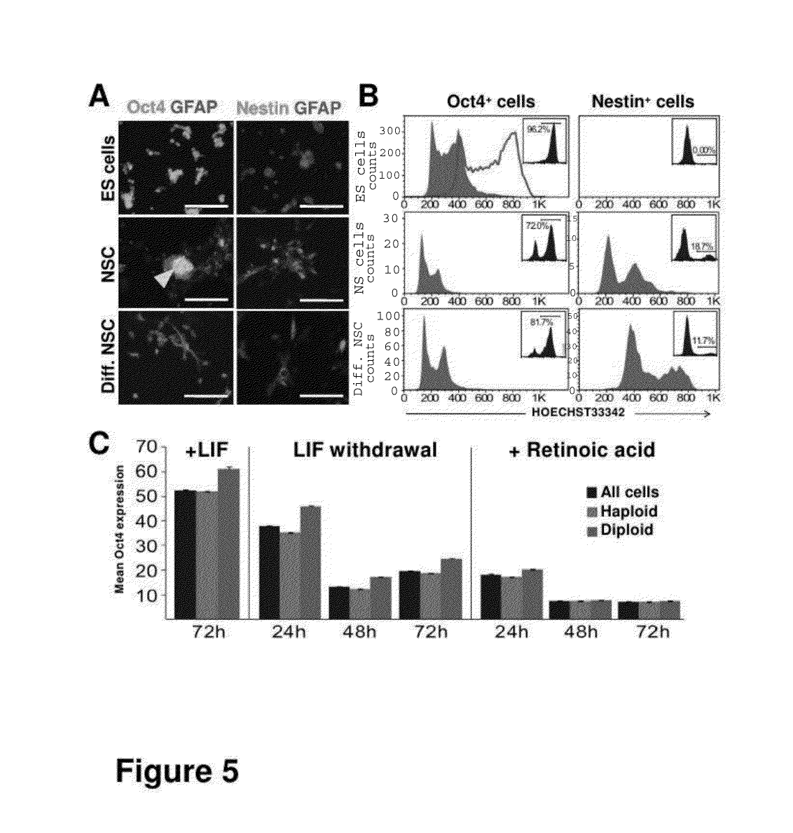

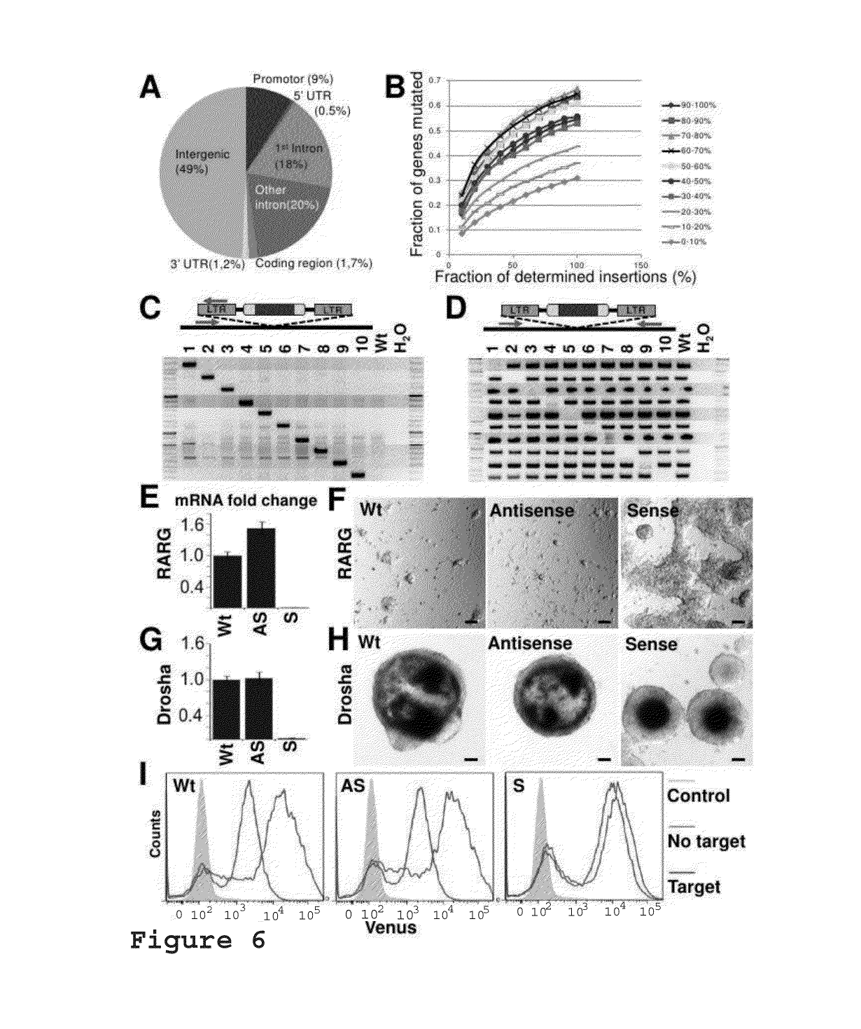

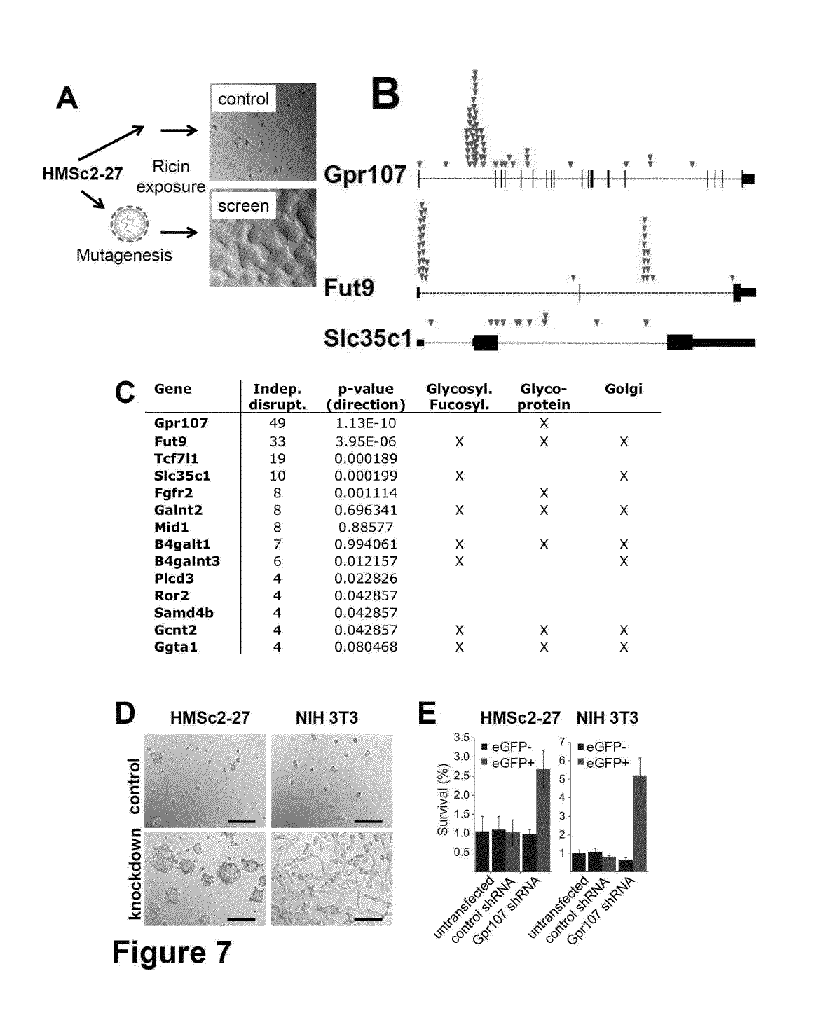

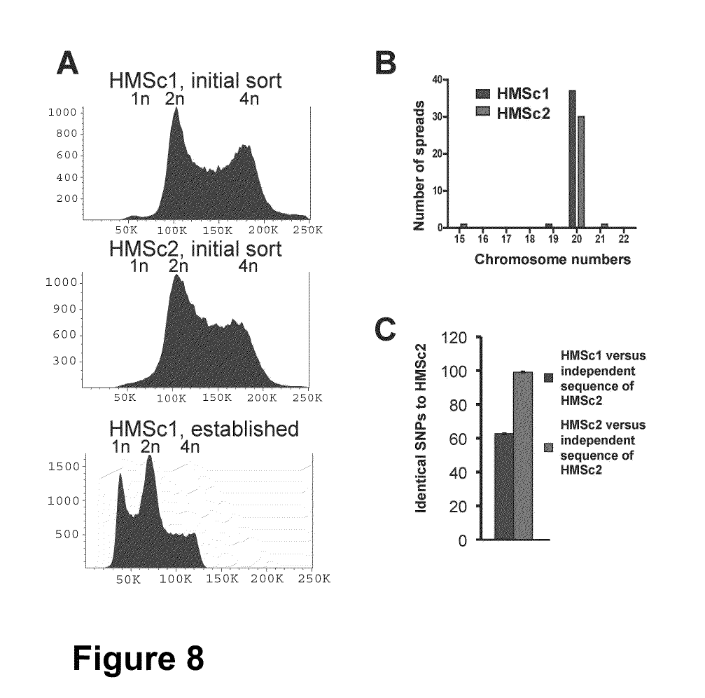

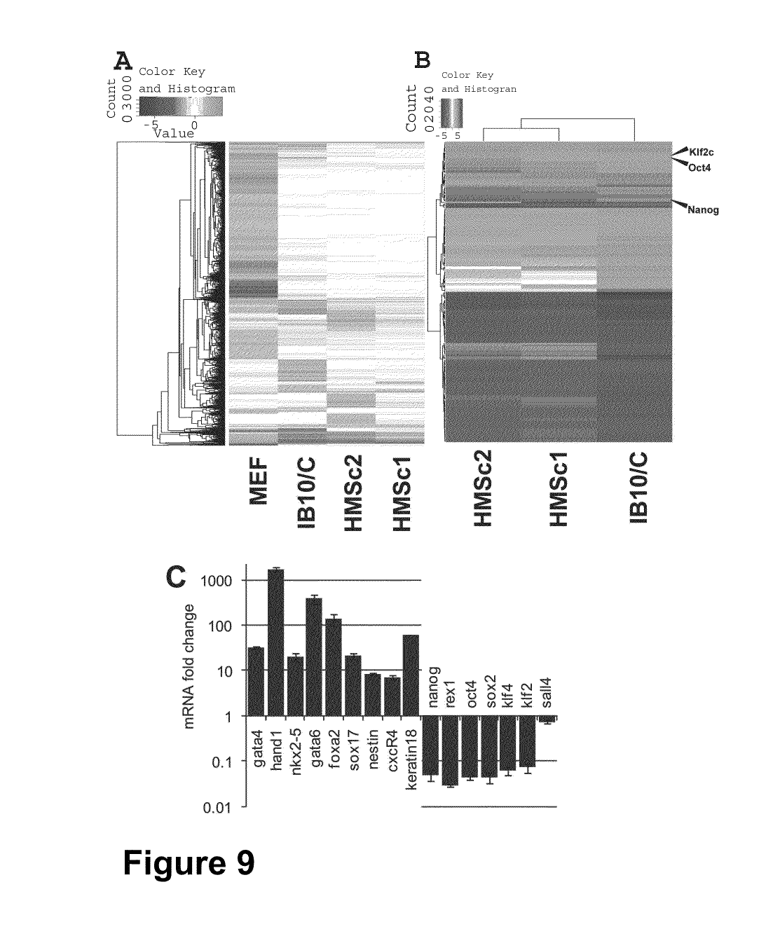

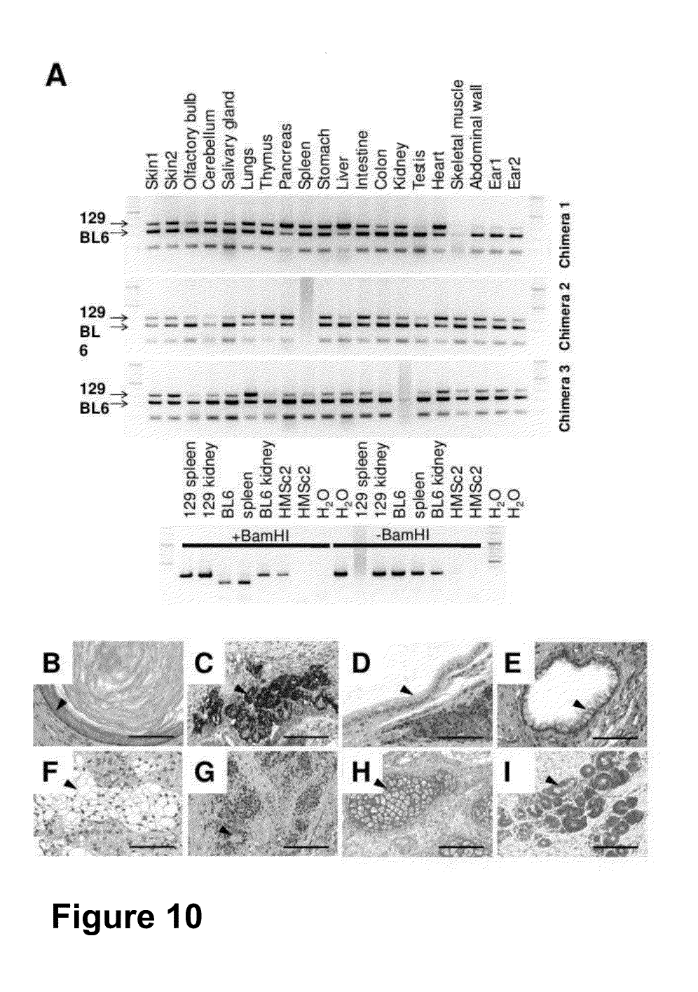

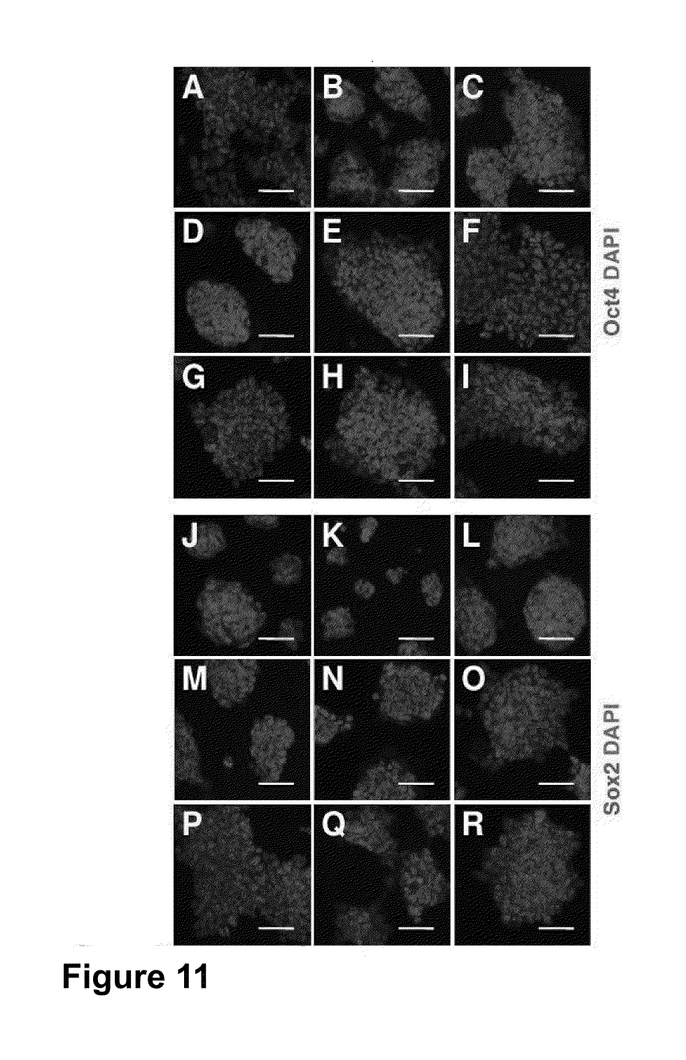

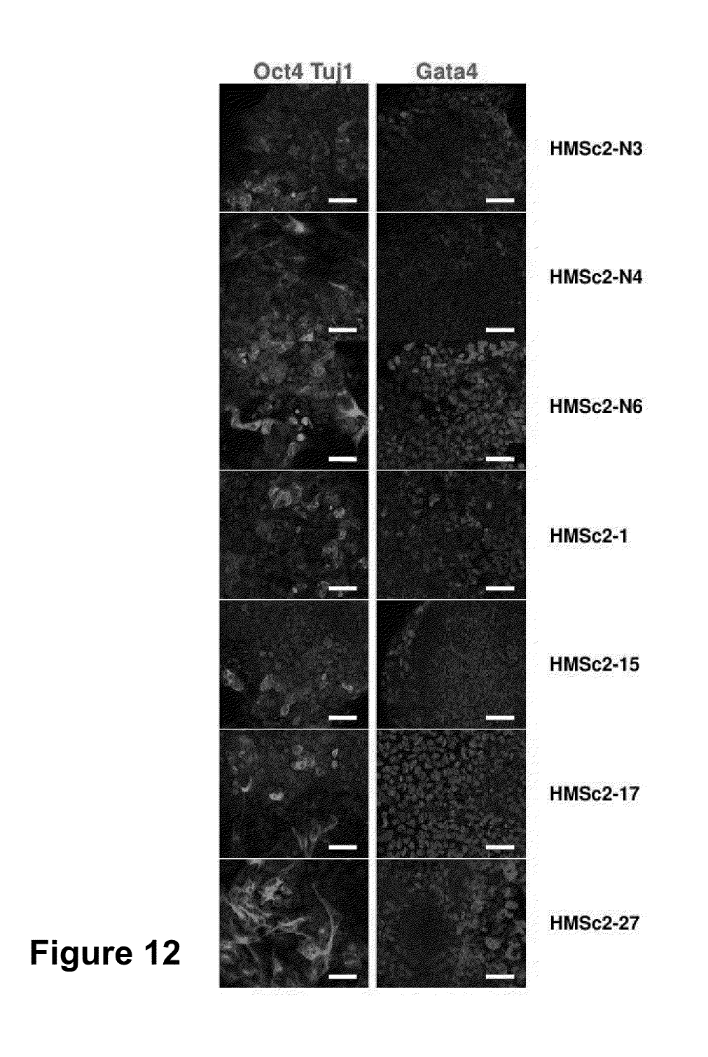

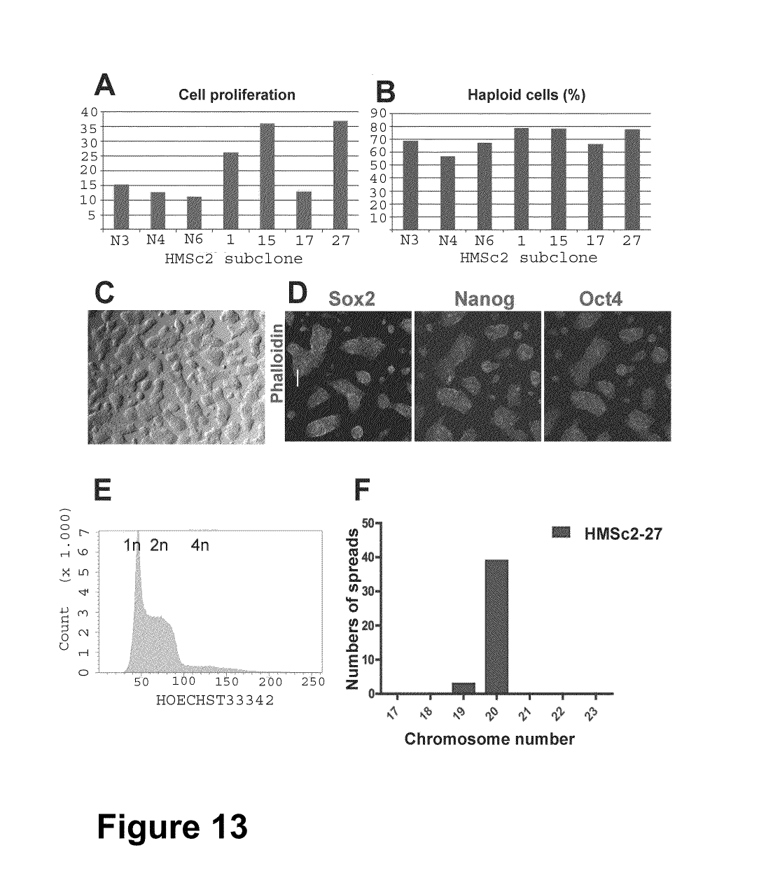

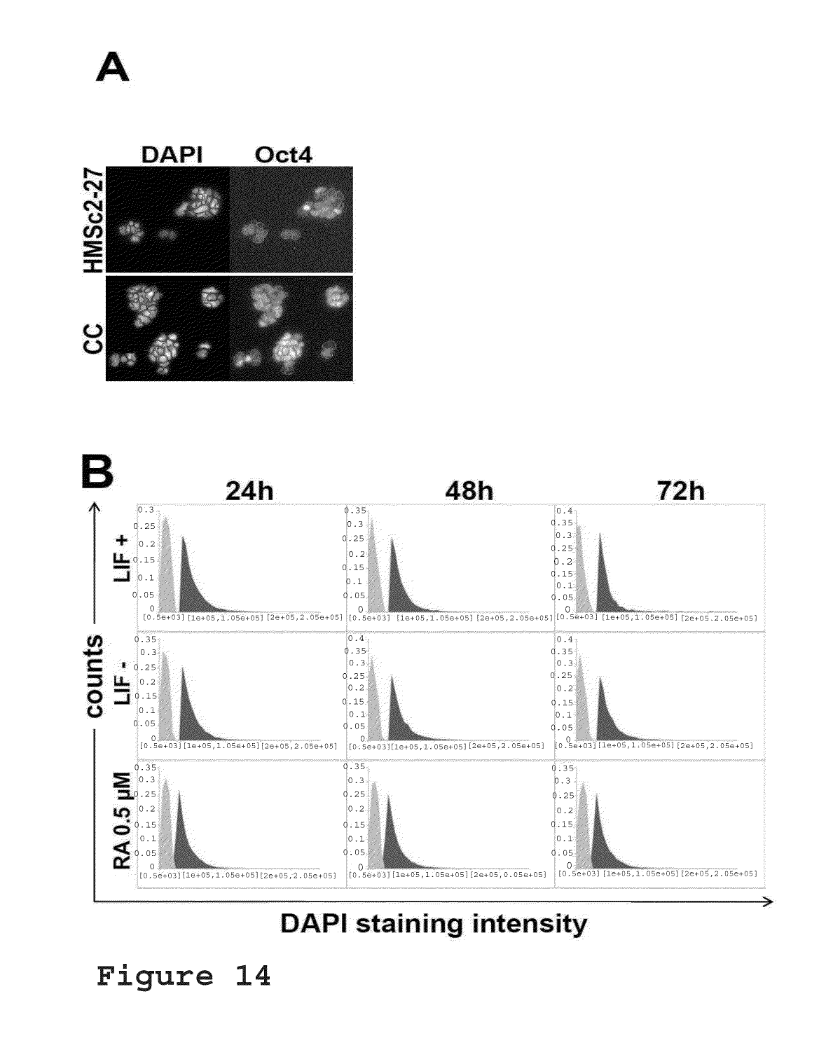

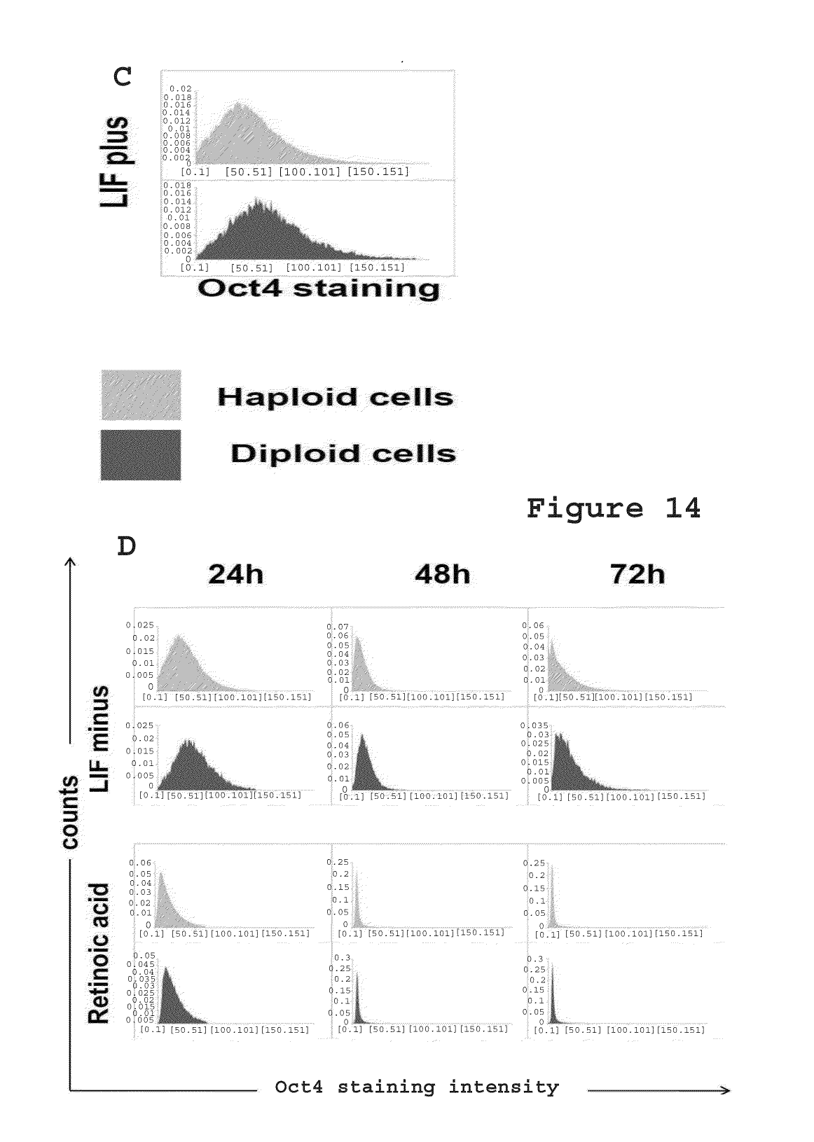

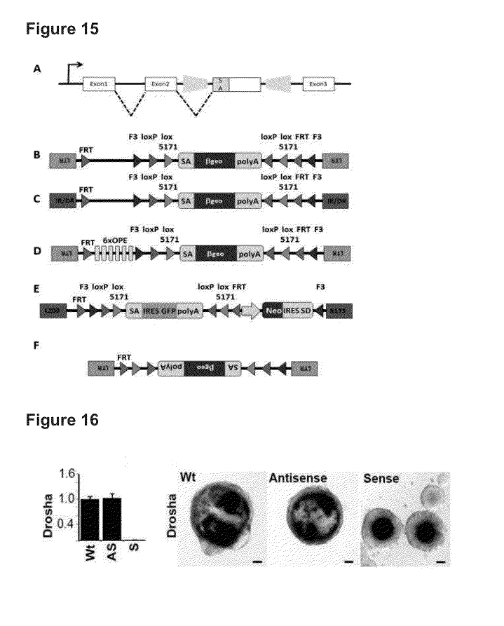

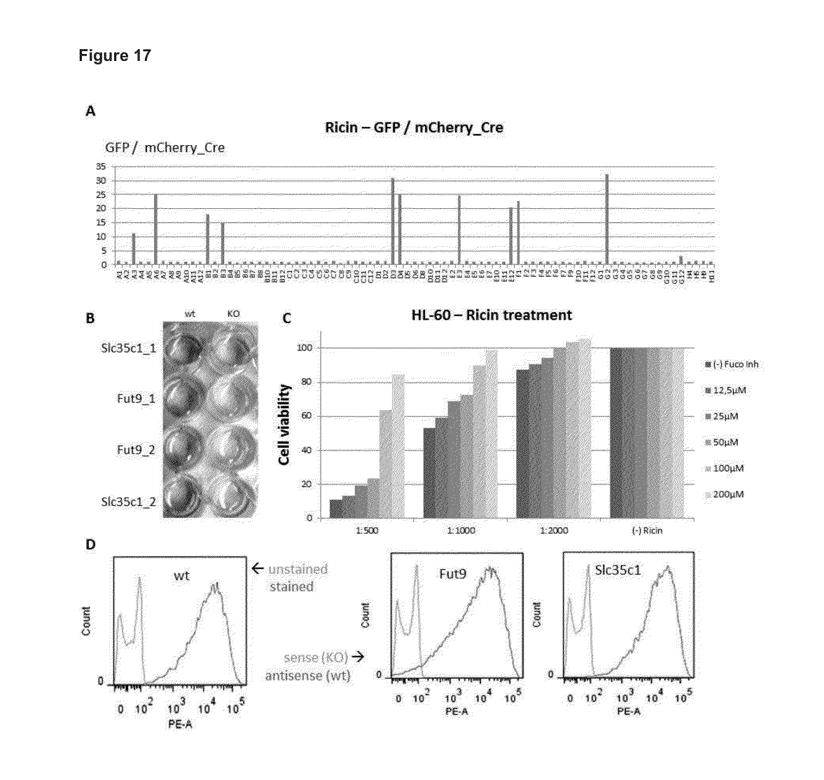

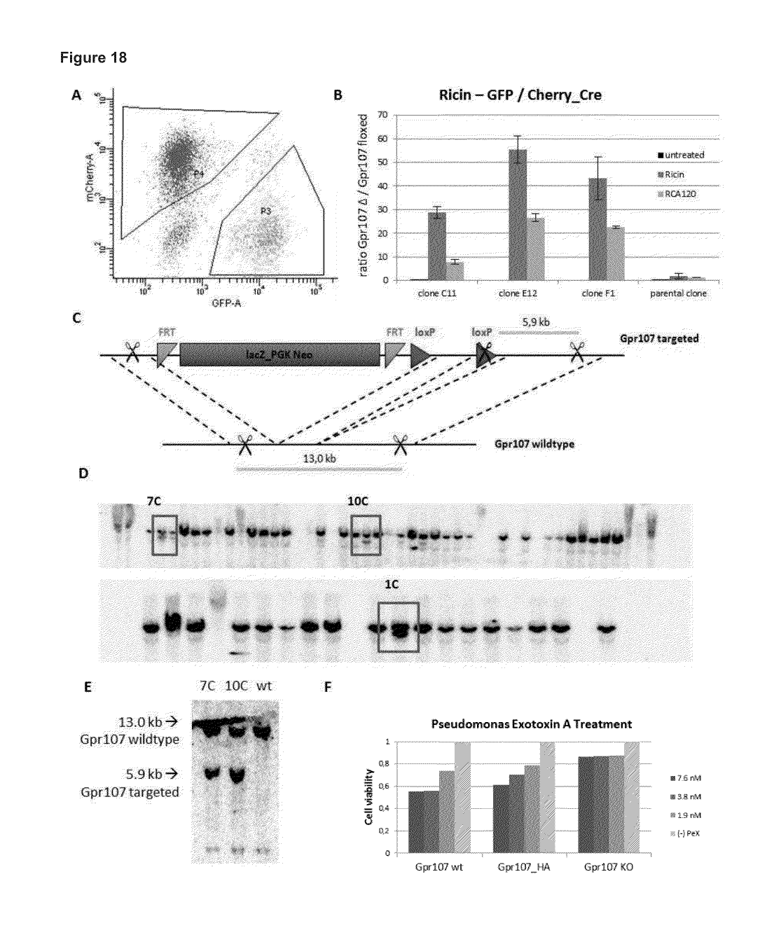

FIG. 8. DNA Content Analyses During Derivation of Haploid Cells, Chromosome Spread, and SNP Analyses; Refers to FIG. 1.

A) The first FACS analysis upon derivation of our two cell lines showed a small subpopulation of haploid cells in G1 of cell cycle (1n) derived from parthenogenotes. The initial sorts are shown. Upon repeated FACS purification (sort 7 is shown), a population of HMSc1 was enriched for haploidy (bottom panel). Of note, since HMSc1 cells always exhibited a larger number of diploid cells we primarily focused on HMSc2 cells. B) The vast majority of chromosome spreads displayed a precisely haploid genome (20 chromosomes). Rare spreads with changed chromosome number can be due to overlapping or washed off chromosomes. C) SNP comparison between HMSc1 or HMSc2 to an independent sequencing run of HMSc2 based on discriminatory SNPs between the 129 and C57BL/6 mouse strains. Haploid cell lines were derived from C57BL/6×129 F1 intercrosses. Data are shown as identical SNPs to HMSc2 confirming genetic independence of the two haploid clones (Student's t-test: p<5.788E-08).

FIG. 9. Transcriptome Analysis and Lineage Markers in Embryoid Bodies; Refers to FIG. 2.

A) Haploid HMSc1 and HMSc2 display a gene expression profile that clusters with that of an established control ES cell line (diploid IB10/C ES cells). The transcriptome profile of MEFs is shown as control. Values are relative to the reference pool of all 4 RNA samples. B) Clustering shows that the transcriptional profile of both haploid HMSc1 and HMSc2 cells closely resembles that of diploid IB10/C ES cells. For clustering, the 100 most up- or down-regulated genes between MEFs and diploid IB10/C stem cells were selected. Three prototypical ES cell genes, namely Nanog, Oct4, and Klf2 are indicated. Supplementary Table 2 lists the 100 genes included in the analysis. For both A) and B), upregulated genes are shown in blue, downregulated genes are shown in red (see color keys), and hierarchical clustering of genes is shown as tree on the side of the heat maps. C) qPCR analysis reveals down-regulation of the stem cell markers Nanog, Rex1, Oct4, Sox2, Klf2, Klf4, and Sall4 in EBs (analysed on day 7) derived from the haploid stem cell line HMSc1 accompanied by expression of the indicated lineage commitment markers (see text). mRNA expression was normalized to undifferentiated haploid stem cells (set at 1).

FIG. 10. Haploid Stem Cells can Contribute to Various Tissues in Adult Mice and Teratomas; Refers to FIG. 3.

A) Contribution of the HMSc2 derived cells to multiple tissues was determined by PCR followed by BamHI digest using primers GAATGTGAGCGCACAGGGTGATGTGCC (SEQ ID NO: 1) and CCCACAGAACACAGTCACAGGGTCC (SEQ ID NO: 2). The indicated tissues were harvested from mice displaying coat color chimerism, processed, and analysed for the presence C57BL6 (BL6) and 129 specific bands. The presence of a 129 band indicates tissue contribution by HMSc2 cells. The specificity of the primers in the BamHI digests are shown in the bottom panel. B-I) Histological examination of teratomas stained with hematoxylin & eosin (B-G) or Alcian blue, counterstained with alizarin red (H,I). Arrowheads point towards B) keratinized stratified epithelium, C) pigmented epithelium, D) ciliated respiratory epithelium, E) glandular tubules with goblet cells, F) adipocytes, G) neurotubules, H) cartilage tissue, and I) sweat gland structures. Scale bars are 100 μm.

FIG. 11. Oct4 and Sox2 Expression on Haploid Stem Cell SubClones; Refers to FIG. 4.

Immunostaining for Oct4 protein expression (red, top panels) and Sox2 protein expression (red, bottom panels) on 9 different subclones that were established by plating single haploid cells directly after FACS purification. Subclones were derived from both HMSc1 and HMSc2 haploid stem cells. The subclones HMSc1-N1 (A, J), HMSc1-N3 (B, K), HMSc2-N3 (C, L), HMSc2-N4 (D, M), HMSc2-N6 (E, N), HMSc2-1 (F, O), HMSc2-15 (G, P), HMSc2-17 (H, Q) and HMSc2-27 (I, R) were seeded on gelatin coated coverslips and immunostained for Oct4 and Sox2 expression. Data are from cells that were subcloned after >30 passages of the parental line confirming stability of expression of bona fide stem cell markers. Cells were counter-stained with DAPI (blue). Scale bars are 50 μm.

FIG. 12. Developmental Potential of Haploid Stem Cell SubClones; Refers to FIG. 4.

Immunostaining for Oct4 (red) and Tuj1 (green) expression and expression of the endodermal marker Gata4 (red) in attached embryoid bodies (EBs, day 10) derived from the indicated subclones. Cells were counterstained with DAPI (blue). Data are from cells that were subcloned after >30 passages of the parental line. Scale bars are 50 μm.

FIG. 13. Proliferation and Stability of the Haploid Cell Fraction in Different Stem Cell Subclones; Refers to FIG. 4.

A) Proliferative kinetics was monitored in 7 subclones derived from haploid HMSc2. 1×10⁶ cells were plated on a 10 cm dish and cell numbers were determined 72 hours later. All clones displayed robust growth. Clones HMSc2-15 and HMSc2-27 reached >30 million cells within a 72 hour timeframe while the HMSc2-N4 and HMSc2-N6 completed more than 1 cell cycle less. B) Percentages of haploid cells present in the populations of seven different subclones derived from the haploid HMSc2 stem cell line. Upon FACS purification of a purely haploid population for all subclones (1n peak as defined by Hoechst33342 histogram), relative populations of haploid versus diploid cells were determined after ten days of culture by FACS analysis on DNA content and quantified using the ModFit software. C) Morphology of the HMSc2 subclone HMSc2-27 cultured under feeder cell free conditions. HMSc2-27 cells remain a large haploid fraction for several weeks even without FACS sorting and exhibit the typical morphology of stem cells forming rounded, compact colonies. Representative DIC (differential interference contrast, Nomarski) images are shown. D) Expression of the prototypical murine embryonic stem cell markers Oct4, Nanog, and Sox2. Note absence of Phalloidinpositive feeder cells. Nanog and Oct4 were co-stained and are shown separately in the red channel. Scale bar: 50 μm. E) Flow cytometry of DNA content in HMSc2-27 cells. DNA content was determined using Hoechst33342. The 1n and 2n chromosomes for haploid and 2n and 4n chromosomes for diploid stem cells are indicated. F) Chromosome spreads of HMSc2-27 cell confirming a haploid genome.

FIG. 14. Separation of Haploid and Diploid Cells Using High Content Imaging Analysis and Quantitative Assessment of Oct4 Levels; Refers to FIG. 5.

A) Representative high content scanning images of haploid HMSc2-27 and diploid CCE cells from 24 hour LIF withdrawal cultures. Parallel cultures of haploid HMSc2-27 and diploid CCE cells were used to develop an algorithm to distinguish haploid versus diploid cells on nuclear area (nuclear mask) and DNA content (DAPI staining intensity). The nuclear masks defined by DAPI staining are depicted in blue. Orange masks were rejected as nuclei from the algorithm as they are too small. The green lines in the Oct4 stained section cells depict nuclear masks as defined by DAPI staining. Of note, as expected, staining is less intense in the haploid cells for both DAPI and Oct4. B) Relative frequency plots showing separation of haploid (yellow) and diploid (blue) HMSc2-27 cells as a function of total DAPI intensity (x-axis) in the presence of LIF (top panels), the absence of LIF (bottom panels), and following treatment with 0.5 μM retinoic acid (bottom) at 24 h, 48 h and 72 h. To avoid inclusion of diploid cells in the haploid group and vice versa, only cells lying below the means of haploid controls were considered haploid, and only cells lying above the means of diploid controls are considered diploid by our algorithm. C) Baseline Oct4 expression in haploid versus diploid HMSc2-27 ES cells cultured in the presence of LIF. D) Oct4 expression of haploid versus diploid HMSc2-27 stem cells cultured in absence of LIF or in the presence of 0.5 μM retinoic acid to induce differentiation. Representative frequency histograms of Oct4 intensity corresponding to the data in FIG. 5C are shown. Note that at the time-points analysed haploid cells (yellow) display nearly identical differentiation dynamics to control diploid cells (blue). Oct4 intensity decreases over time under both differentiation conditions but particularly rapidly under retinoic acid treatment. Note that Oct4 expression decreases in haploid as well as diploid HMSc2-27 cells at comparable rates upon initiation of differentiation.

FIG. 15. Vectors Used for Insertional Mutagenesis.

SA, splice acceptor site; SA, splice donor, site IRES, internal ribosomal entry site; GFP, Green fluoresent protein; Neo, G418 selection cassette; βgeo, lacZ-neomycin fusion gene; LTR, Long terminal repeat; LoxP and lox5171, “wild type” and mutated LoxP sites; FRT, Flp recombinase recognition target; F3, variant Flp recognition site that recombines with other F3s, but not the heterotypic type FRT site. R175, L200, Tol2 transposon terminal essential sequences; OPE, Osteopontin enhancer element, Oct4 binding; IR/DR Inverted/Direct Repeat of sleeping beauty transposon.

FIG. 16. PCR Analysis of Drosha mRNA in Haploid ESCs that are Wild-Type (Wt), or Contain the Splice Acceptor in Antisense (AS) or Sense Orientation (S)

mRNA was normalized to parental ESCs. Mean+/−SD. Note complete absence of cystic EBs in Drosha-deficient ESCs compared to Wt ESCs and ESCs containing the splice acceptor in antisense. Representative EBs are shown (day 10). Bars: 100 μm.

FIG. 17

A Ratio of GFP to mCherry Cre expressing cells under ricin treatment compared to control cells; cells with ricin resistance mutation show an increase viability in the range of 10 to 33 fold (bars).

B Reversion of the ricin resistant phenotype of valid clones through Cre recombinase expression and reconstitution of wildtype locus; all wild type (wt) cells die upon ricin challenge (red).

C Cell viability of human HL-60 cells under Ricin toxicity upon treatment with fucosylation Inhibitor (Fuco Inh); bars from left to right: increasing Fuco Inh concentration; bar blocks from left to right: decreasing ricin concentration; Different dilutions of a Ricinus communis seed extract (1:2000, 1:1000, 1:500 and nor ricin) have been used.

D Expression of the sugar epitope SSEA-1 in Fut9 and Slc35c1 cells upon inversion of the targeting cassette (anti SSEA-1-PE staining)

FIG. 18

A FACS blot of GFP and mCherry expression pattern of virus transfected cells

B Ratio of GFP to mCherry Cre expressing cells under ricin treatment compared to control cells

C Conditional gene targeting cassette of Gpr107

D Screening for homologous recombination via southern blotting

E Confirmation of germline transmission through southern blot analysis

F Comparison of the cell viability of Gpr107 wildtype, reconstitution and knock out cells under Pseudomonas Exotoxin treatment; bars from left to right: decreasing Pseudomonas Exotoxin (PeX) concentration; Knock out-cells (Gpr107 KO) show higher cell viability; knock-out and reconstitution of a Gpr107 gene (Gpr107 HA) resotre the wild type (wt) phenotype.

### EXAMPLES

The data herein shows that it is possible to generate mammalian haploid stem cell lines from parthenogenetic mouse blastocysts derived from ethanol or strontium activated oocytes. Detailed molecular characterization of the haploid stem cells shows that these cells express all classical markers of diploid ES cells, carry 20 chromosomes, and largely maintain genome integrity. Functionally, these haploid stem cells can differentiate into cells from all three germlayers in vitro and in vivo. Although our lines and subclones are stable and in some cases have been maintained for over 70 passages, some haploid cells become diploid. The mutagenesis data suggest that these cells do not become diploid via cell fusion, but rather via failed cytokinesis and/or endoreplication of the genome. Most importantly, the haploid stem cells can be mutated and, in all cases, these mutations are homozygous indicating that such haploid cells can be used to analyse recessive and disease phenotypes in various cell lineages in vitro. Although contribution of the cell lines to multiple tissues was detected in vivo in chimeras. Germline transmission could be attempted using semi-cloning techniques as previously reported for Medaka (Yi et al., 2009).

These results allow to combine the power of a haploid genome with pluripotency of embryonic stem cells. Recessive genetic screens have elucidated a wide variety of biological processes over the last century and thus markedly contributed to the understanding of normal development, basic physiology, and disease. Reverse genetics is feasible using a vector system that provides immediate confirmation of gene function in the same clones using Cre mediated conversion of the splice acceptor sites. Using this system the inventors have indeed been able to functionally validate our approach using clones with conditional Rarg and Drosha mutations. Moreover, a forward genetic screen was performed for ricin toxicity, one of the most dangerous poisons also being used/investigated as a biological weapon by governments. Ricin also gained notoriety for its potential use as toxin in bioterrorism. This screen in haploid stem cells identified the GPCR Gpr107 as an essential molecule required for ricin induced killing. Since no antitoxins are available for treatment of ricin poisoning, molecular inhibition of Gpr107 (and other molecules identified by our screen) are expected to be useful to alleviate ricin toxicity in vivo.

### Summary of Methods

### Parthenogenetic Activation of Oocytes

Oocytes were collected and incubated in 5% Ethanol for 5 minutes. Subsequently, viable oocytes were transferred into pseudopregnant mice and blastocysts were collected at embryonic day 3.5.

### Derivation of ES Cells

Blastocysts were placed on a feeder coated cell culture dish with ES cell derivation medium as previously described (Bryja et al., 2006). Blastocyst outgrowths were trypsinized and replated on a feeder layer to allow for ES cell colony outgrowth.

### Genomic Sequencing

Purified DNA from mouse kidney or ES cells was sheared by sonication and subjected to the Illumina adaptor ligation protocol and sequenced in Illumina HiSeq. Reads were mapped to the genome using bowtie.

### Stem Cell Characterization

Cells were analyzed for the presence of stem cell markers using qPCR or immunofluorescent labeling as well as an enzymatic assay for alkaline phosphatase.

### Differentiation

Cells were differentiated by withdrawal of LIF and EB formation or by direct neuronal differentiation (Pollard et al., 2006). For teratoma formation, cells were injected testicular or subcutaneously into nude mice and teratoma growth was monitored. To generate chimeric mice, HMSc2 ES cells were injected into C57BL/6 ED3.5 blastocysts, and transferred into pseudopregnant females. Percentage chimerism was determined by coat colour.

### Retroviral Infection of ES Cells and Inverse PCR

Virus was packaged in Platinum E cells. Selection for gene trap insertions was done using G418. The protocol for inverse PCR was adapted based on Carette et al. (Carette et al., 2011).

### Ricin Toxicity Screen

A mutated library of cells was exposed to a lethal dose of ricin for 3 weeks. DNA of surviving cells was purified, subjected to inverse PCR and analyzed by deep sequencing. For confirmation using shRNA knockdowns, cells were exposed to ricin for 48 hours and survival was quantified using FACS.

### Example 1

### Methods

### Example 1.1

### Parthenogenic Derivation of Haploid Stem Cells

C57BL/6×129 F1 female mice were super-ovulated using standard protocols and unfertilized oocytes were flushed and collected. For activation, oocytes were exposed to 5% ethanol or 25 mM SrCl₂ as described (Kaufman et al., 1983; Otaegui et al., 1999). Four hours post activation, viable oocytes were transferred into pseudopregnant 129 females, re-collected on embryonic day (ED) 3.5 and cells were derived according to established embryonic stem derivation protocols (Bryja et al., 2006). Parthenogenetically derived stem cells were initially maintained on feeder layers and subsequently adapted to feeder cell free culture conditions. Haploid cells were trained to grow under feeder free conditions by gradually reducing feeder cell density and eventually removing the feeder cells from the culture. Stem cell medium consisted of DMEM with 15% FCS (Gibco), supplemented with 2 mM L-Glutamate, 1 mM sodium pyruvate, 100 U penicillin/ml, 0.1 mg streptomycin/ml, 1× non essential aminoacids, 50 mM beta-mercoptoethanol (all Sigma) and ESGRO at 1,000 U/ml (Millipore).

### Example 1.2

### Genome Coverage Analysis and SNP Mapping

Genomic DNA preparations were sheared using a Covaris DNA sonicator, adaptor ligated, and subjected to Illumina sequencing (HiSeq) according to the manufacturers protocol. Reads were mapped using Bowtie (allowing for up to 3 mismatches and requiring that reads map to a single genomic position) and coverage was analysed as reads/50 kb window relative to coverage in the parental strains 129 and C57BL/6, considering only unique genomic coordinates. SNPs were retrieved that differ between C57BL/6 and 129 from Sanger (www.sanger.ac.uk/resources/mouse/genomes/) and mismatches observed during genome mapping of the deep sequencing reads against them were evaluated. Each SNP that was covered by the Solexa reads was assigned to C57BL/6 and 129 according to the majority of reads.

### Example 1.3

### Alkaline Phosphatase Activity, Immunohistochemistry, and Chromosome Spreads

Alkaline phosphatase activity was detected using VECTOR kit SK-5300. Chromosome spreads were performed following established protocols (Nagy et al., 2008). Immunofluorescence of cultured cells or embryoid body (EB) cultures, neural stem cell, and differentiated neural stem cell cultures was performed after fixation in 4% PFA for 1 h, blocking and permeabilization in PBS supplemented with 1% Glycine, 2% BSA, 0.2% Triton, and 5% FCS for 1 h. Cells were incubated with the primary antibody o/n at 4° C. (anti-Nestin, Abcam 6142, 1:300; anti-Gata4, Santa Cruz, 1:500; anti-Oct3/4, BD Transduction, 1:100; anti-Tuj1, Covance RB-435P, 1:1000; anti-cytokeratin 5, PRP160P-100, Covance; anti GFAP, DAKO, 1:200; anti-Sox2, Cell Signaling, L1D6A2 mouse mAB 1:100; anti-Nanog, Abcam ab80892, 1:100), washed, incubated with fluorescent labeled goat secondary antibodies (Molecular Probes) and visualized using a Zeiss Axioplan2 (neural stem cell differentiation), or Zeiss LSM700 confocal microscope (embryoid bodies). For analyses of in vivo differentiation, teratomas were collected, fixed o/n in 4% PFA, paraffin embedded, sectioned, and stained with haematoxylin and eosin (H&E), Alcian blue and nuclear fast red, or processed for immunodetection of Nestin and Cytokeratine 5. Primary Abs were detected using biotinylated secondary antibodies. Ab staining was visualized using streptavidin-HRP and DAB and sections counterstained using hematoxylin. Immunohistochemistry was performed using the Ventana automated system. Images were collected using Zeiss miraxscan. Of note, teratomas were assessed by a certified pathologist.

### Example 1.4

### Flow Cytometry

For FACS analyses, cells were trypsinized, washed, and then incubated with 10 μg/ml Hoechst33342 while pre-plating for 30 minutes. Subsequently, cells were collected by centrifugation, and FACS sorted for DNA content (as well as FSC-A and SSC-A) using BD FACSAriaIII. Intracellular staining (for FACS subsequent to trypsinization) was performed using the same primary and secondary antibodies as described for immunohistochemistry staining.

### Example 1.5

### Gene Expression Analyses

RNA was purified using QIAgen RNeasy Mini Kit. Reverse transcription, DNA labeling and microarray hybridization was done according to the manufacturers protocols (Agilent) using the x44K Mouse Genexpression Array DesignID 14868. For qPCR analyses, RNA was purified using QIAgen RNeasy Mini Kit and reverse transcribed using the iScript Kit (Biorad). Amplification was monitored with iQ SYBR Green supermix using the iQ5 Real Time PCR detection System (Biorad). Expression levels were calculated using the ΔΔct method with Gapdh as housekeeping gene. The following PCR primers were used:

| | | |
|---|---|---|
| nanog | GCAAGAACTCTCCTCCATT | forward |
| | (SEQ ID NO: 3) | |
| | ATGCGTTCACCAGATAGC | reverse |
| | (SEQ ID NO: 4) | |
| oct-4/ | TCACTCACATCGCCAATC | forward |
| pou5f1 | (SEQ ID NO: 5) | |
| | CCTGTAGCCTCATACTCTTC | reverse |
| | (SEQ ID NO: 6) | |
| sox2 | CTCGCAGACCTACATGAAC | forward |
| | (SEQ ID NO: 7) | |
| | CTCGGACTTGACCACAGA | reverse |
| | (SEQ ID NO: 8) | |
| klf4 | TCTCTCTTCTTCGGACTCC | forward |
| | (SEQ ID NO: 9) | |
| | CTGGACGCAGTGTCTTCT | reverse |
| | (SEQ ID NO: 10) | |
| c-myc | GTACCTCGTCCGATTCCA | forward |
| | (SEQ ID NO: 11) | |
| | CATCTTCTTGCTCTTCTTCAG | reverse |
| | (SEQ ID NO: 12) | |
| sall4 | AACTTCTCGTCTGCCAGT | forward |
| | (SEQ ID NO: 13) | |
| | GAGTCATGTAGTGTACCTTCA | reverse |
| | (SEQ ID NO: 14) | |
| kfl2 | CTCAGCGAGCCTATCTTG | forward |
| | (SEQ ID NO: 15) | |
| | AGAGGATGAAGTCCAACAC | reverse |
| | (SEQ ID NO: 16) | |
| gata-4 | GTGAGCCTGTATGTAATGC | forward |
| | (SEQ ID NO: 17) | |
| | CTGCTGGCGTCTTAGATT | reverse |
| | (SEQ ID NO: 18) | |
| hand1 | CCTTCAAGGCTGAACTCA | forward |
| | (SEQ ID NO: 19) | |
| | CGCCCTTTAATCCTCTTCT | reverse |
| | (SEQ ID NO: 20) | |
| gata-6 | CTCCTACTTCCTCTTCTTCTAA | forward |
| | (SEQ ID NO: 21) | |
| | CGTCTTGACCTGAATACTTG | reverse |
| | (SEQ ID NO: 22) | |
| foxa2 | GAGCCGTGAAGATGGAAG | forward |
| | (SEQ ID NO: 23) | |
| | GTGTTCATGCCATTCATCC | reverse |
| | (SEQ ID NO: 24) | |
| sox17 | GCCGATGAACGCCTTTA | forward |
| | (SEQ ID NO: 25) | |
| | CAACGCCTTCCAAGACTT | reverse |
| | (SEQ ID NO: 26) | |
| krt18 | TTGCCGCCGATGACTT | forward |
| | (SEQ ID NO: 27) | |
| | CAGCCTTGTGATGTTGGT | reverse |
| | (SEQ ID NO: 28) | |
| zfp42/ | CTGCCTCCAAGTGTTGTC | forward |
| rex1 | (SEQ ID NO: 29) | |
| | GAACAATGCCTATGACTCAC | reverse |
| | (SEQ ID NO: 30) | |
| drosha | CCAAGATGATCCAACTCCTT | forward |
| | (SEQ ID NO: 31) | |
| | GGTGCTGATTCTGAACAATG | reverse |
| | (SEQ ID NO: 32) | |
| rarg | CACCATTTGAGATGCTGAG | forward |
| | (SEQ ID NO: 33) | |
| | GGCTTATAGACCCGAGGA | reverse |
| | (SEQ ID NO: 34) | |

### Example 1.6

### Differentiation of Stem Cells, Teratoma Formation, and Chimeric Mice

For embryoid body (EB) formation, stem cells were trypsinized and cultured in absence of LIF either in hanging drops or in bacterial dishes. For retinoic acid (RA) induced differentiation, cells were grown in presence of 0.1 μM RA for 1 week, plated at density of 1 million per 10 cm dish and assayed 72 hours later. For myoblast differentiation, stem cells were cultured in hanging drops for 4.5 days in stem cell medium in absence of LIF and subsequently rinsed onto gelatinized cell culture dishes. Adhering cell aggregated were fed every 3^{rd} day by replacement of 8 ml/10 ml stem cell medium without LIF. Movies of beating myoblasts were recorded on days 11-13 at 36 shots/second using a Zeiss Axiovert 200M and a CoolSNAP HQ². Derivation of neuronal stem cells and further differentiation of neuronal stem cells into GFAP⁺astrocytes and Tuj1⁺neurons was performed as described (Pollard et al., 2006). For teratoma formation, cells were injected testicular or subcutaneously into nude mice and teratoma growth was monitored. To generate chimeric mice, the diploid fraction of HMSc2 stem cells was purified using flow cytometry, cultivated for 7 days, injected into C57BL/6 ED3.5 blastocysts, and transferred into pseudopregnant 129 females. Percentage chimerism was determined by coat colour.

### Example 1.7

### Retroviral Infection of Stem Cells

Oct4 enhanced gene trap retroviruses carrying a splice acceptor followed by a neomycin resistance gene in 3 reading frames and Oct4 binding sites to enhance transcription (Schnutgen et al., 2008) were packaged in Platinum E cells (Cell Biolabs), concentrated by centrifugation (25,000 rpm, 4° C., 4 h) and applied to stem cells with 2 μg polybrene per ml for 8 hours. Selection for gene trap insertions was done using G418 (Gibco) at 0.2 mg/ml. To estimate numbers of integrations 500,000 cells were plated on 15 cm dishes, selected for integrations using G418 selection and colonies counted after 10 days. For comparison, 5,000 cells were plated without selection.

### Example 1.8

### Inverse PCR

The protocol for inverse PCR was adapted based on Carette et al (Carette et al., 2011). In brief, genomic DNA preparations were digested using DpnII or MseI, purified using the QIAquick Gel Extraction Kit, and fragments ligated at a concentration of 3 μg/ml over night. The ligase was then heat inactivated and rings were re-digested in ligase buffer using the enzymes NheI and PvuII. Linearized fragments were purified using the QIAquick Gel Extraction Kit and subjected to PCR using Accuprime Taq polymerase (Invitrogen), primers FS Solexa upstream and FS Solexa downstream, and a BioRad Thermal Cycler. The program of 95° C. for 30 sec, 60° C. for 30 sec, and 68° C. for 105 sec was repeated 36 times. Amplicons were loaded on agarose gels, eluted and subjected to deep sequencing using an Illumina Genome Analyzer and primer FS flowcell. The following iPCR primers were used:

upstream primer
(SEQ ID NO: 35)
AATGATACGGCGACCACCGAGATCGCCAGTCCTCCGATTGA
downstream primer
(SEQ ID NO: 36)
CAAGCAGAAGACGGCATACGAGTTCCTATTCCGAAGTTCCTATTCTCTA
flowcell sequencing primer
(SEQ ID NO: 37)
TGATTGACTACCCGTCAGCGGGGGTCTTTCA

### Example 1.9

### Mapping of Viral Integration Sites

Solexa reads were mapped to the mouse genome using Bowtie and requiring a unique best match to the genome. ENSEMBL gene annotations were used to determine the fraction of integration sites in introns, exons, UTRs, promoters (defined as 2 kb upstream of the transcriptional start site), and the remaining intergenic regions. ENSEMBL transcripts were split into 10 bins according to their expression levels in our haploid stem cells as measured by their absolute signal on the Agilent array used for transcriptome analysis and the fraction of transcripts with viral integrations was assessed in each bin. For the equivalent gene-based analysis, the most highly expressed transcript was considered for each gene. The analysis was repeated to estimate the coverage of viral integrations with sub-samples of the total insertions sites.

For confirmation of mapped integration sites the following primers were used:

| | |
|---|---|
| madcam1-F | AGTCTCTCCTTTGCCCTGCTACTGG |
| | (SEQ ID NO: 38) |
| madcam1-R | CACAGGCATTGAACAGTTTTGTTGG |
| | (SEQ ID NO: 39) |
| drosha-F | TTCGAGTTATAGACTGTAATGAGCC |
| | (SEQ ID NO: 40) |
| drosha-R | CCTACACTCTCTAGCAACGGAAGCC |
| | (SEQ ID NO: 41) |
| RARG-F | GCTGTTGTCACCCTTGTGCAT AAGCC |
| | (SEQ ID NO: 42) |
| RARG-R | AGATGCTGGGAATGGAACCCTGGTCC |
| | (SEQ ID NO: 43) |
| Ap4s1-F | GTAGCTTAGAAACTCTGGCCACTGG |
| | (SEQ ID NO: 44) |
| Ap4s1-R | CAGTGAAGTCTGAATACAGAGAATGG |
| | (SEQ ID NO: 45) |
| Arap1-F | GTCCATGCAGGTTTGAGTGACTCC |
| | (SEQ ID NO: 46) |
| Arap1-R | GACCTCCAGCTACAGAGGACAGAGCC |
| | (SEQ ID NO: 47) |
| Evx1-F | TGTCAAGGGCAAGAGCTGCGAAGG |
| | (SEQ ID NO: 48) |
| Evx1-R | CCAATGTCAAACCGGAAGGGAGAAGG |
| | (SEQ ID NO: 49) |
| Bc1211-F | GAGTTACAGATGACTGCGAGCTGCC |
| | (SEQ ID NO: 50) |
| Bc1211-R | GAAGCATTGAGTAGCTTTACCTGCC |
| | (SEQ ID NO: 51) |
| 2210012G12Rik-F | GTAGACCTGACTTGACTGGCTTGG |
| | (SEQ ID NO: 52) |
| 2210012G12Rik-R | GATGCTCATCTTACCAAACGCATCTC |
| | (SEQ ID NO: 53) |
| Tit-F | CTTCGACCGTCTGGTCCTCAAGAGG |
| | (SEQ ID NO: 54) |
| Tit-R | GAAACCAGCCTGATCTACATAGTGG |
| | (SEQ ID NO: 55) |
| chr2:50928851-F | ACTTCCGACAAGAT TCTCAGTCC |
| | (SEQ ID NO: 56) |
| chr2:50928851-R | CGTGACCTTTGGGTGTGTAATGCC |
| | (SEQ ID NO: 57) |

### Example 1.10

### Protein Quantification and Differentiation in Single Stem Cells

Differentiation analysis was carried out using a modification of high content screening (HCS) protocols we have previously published analysing the loss of Oct4 expression as a measure of differentiation (Walker et al., 2010; Walker et al., 2007). All HCS experiments were carried out with the feeder free subclone HMSc2-27 at >50 passages. Cells were cultured in parallel in separate rows of 96-well tissue-culture plates (Greiner). Cells were trypsinized to a single-cell suspension and plated at 6000 cells/well into wells that had been pre-coated with a fibronectin/gelatin mixture (12.5 μg/mL fibronectin, Sigma and 0.02% gelatin in water, Millipore). Cells were fixed at 24 h, 48 h, and 72 h time-points with 4% paraformaldehyde for 15 min at room temperature. Immunostaining for Oct4 was carried out as follows: fixed cells were blocked for 30 min at room temperature, permeabilized with 0.1% Triton X-100/PBS for 1 h at 4 C, washed once with permeabilization buffer (PB, 5% FBS and 0.3% Triton X-100 in PBS), then incubated with mouse monoclonal anti-Oct4 antibody (BD, 1:100) for 1 h at room temperature. Then, cells were washed four times with PBS and incubated with anti-mouse IgG1 AlexaFluor488 (Invitrogen, 1:100) for 1 h in a 1:5000 dilution of DAPI in PB. Plates were then imaged on a ThermoFisher Cellomics ArrayScan VTi automated fluorescence microscope. Data were acquired using a minimum of 9 wells for each time point and condition. An algorithm was designed in the R language based upon nuclear size and DAPI intensity (DNA content) to distinguish haploid cells from diploid cells within mixed cultures of the haploid cell line HMSc2-27. To ensure the fidelity of our haploid vs. diploid analyses, the R algorithm called cells “haploid” only if their DAPI intensity was below the mean DAPI intensity of the haploid controls and called cells “diploid” only if their DAPI intensity was above the mean DAPI intensity of the diploid controls (see FIG. 9).

### Example 1.11

### Reversion of the Splice Acceptor Element in the Retrovirus

Clones carrying the gene trap vector were transiently transfected with a plasmid encoding for Cre recombinase as well as GFP, FACS sorted for GFP positive cells, plated at clonal density, picked, expanded and analysed for inversion using PCR analysis.

### Example 1.12

### MicroRNA Sensor Experiments

The pSIN-TRE-dsRedmiR30/shRNA-PGK-Venus-shRNA target site vector was used. This vector is a derivative of pSENSOR that enables fluorescence (dsRed)-based monitoring of shRNA expressing cells (Fellmann et al., 2011). Two variants of this vector harboring a potent shRNA targeting Firefly Luciferase (shLuc.1309) with or without its specific target site in the 3′UTR of Venus were each cotransfected with MSCV-rtTA3-PGK-Puro into control wild type ES cells as well as ES cells with antisense (AS) and sense (S) integrations of the mutagenesis vector using Lipofectamine 2000 according to manufacturers protocols. 8 hours after transfection, transfected cells were treated with doxycyline (1 μg/ml) to induce shRNA expression, and after 48 hours, shRNA expressing (dsRed+) cells were analysed for Venus reporter expression level on a FACS-Aria-III flow cytomter (BD).

### Example 1.13

### Ricin Screen

Ricin crude extracts in cell culture medium was generated as in (Simmons and Russell, 1985) and concentration was titrated to kill all cells efficiently within 3-4 days. In order to identify genes involved in ricin toxicity, we plated 25 million cells of the mutagenized library described above (FIG. 13) in five 15 cm dishes. The library used had a complexity of about 7.5 million different, genetically independent mutations. On a sixth 15 cm plate, 5 million non-mutagenized cells of subclone HMSc2-27 were plated. Cells were maintained in stem cell culture medium in presence of ricin for 2 weeks. At this point, several hundred distinct colonies had appeared on otherwise empty plates while the control plate was completely free of colonies with typical stem cell morphology. To purify ricin insensitive cells further, all cells were trypsinized and replated on an equal surface. Ricin selection in control and library was extended by 1 week. While control plates were entirely free of colonies now, library plates begun to overgrow. We lysed all cells in one pool, purified DNA and subjected the DNA to inverse PCR and deep sequencing (described above) to retrieve all viral integration sites in cells that remained after 3 weeks of constant ricin treatment.

### Example 1.14

### Gpr107 Knockdown

A shRNA targeting Gpr107 was designed (TGCTGTTGACAGTGAGCGCAACTAGCTTATTCATAGCCAATAGTGAAGCCACAGATGTATTGGCTATGAATAAGCTAGTTTTGCCTACTGCCTCGGA, SEQ ID NO: 58) and cloned into LMN (MSCV-miR30-PGK-NeoR-IRES-GFP) as described (Zuber et al., 2010; Zuber et al., 2011). A retroviral vector expressing GPF and shRNA to knock down Gpr107 or a scrambled control shRNA were packaged in Platinum E cells and HMSc2-27 and NIH3T3 cells were transfected in triplicate. After 72 hours, cells were split onto 2 10 cm plates, one plate was left untreated and one plate was treated with ricin (1:250 of crude extract for NIH 3T3, 1:2000 of crude extract for HMSc2-27). Analysis of survival was performed after 48 hours following addition of ricin. Cells were analysed by flow cytometry for absolute number, eGFP expression and viability (propidium iodine staining) using an LSR Fortessa (BD).

### Example 2

### Derivation of Haploid Cell Lines from Parthenogenic Murine Blastocysts

Parthenogenetic embryos develop from haploid oocytes and thus contain only the maternal genome. However, all reported cell lines derived from parthenogenetic embryos carry a diploid set of chromosomes (Kaufman et al., 1983). It was previously not known that haploid cells might still be present in parthenogenetic early embryos and that haploid stem cells could be derived from such blastocysts. To accomplish this, oocytes from superovulated C57BL/6×129 F1 females were activated by exposure to 5% ethanol or 25 mM SrCl₂. Activated oocytes were then transferred into pseudo-pregnant recipients (FIG. 1A). At embryonic day (ED) 3.5, compacted morulae and blastocysts were harvested and cultivated under conditions used to derive stem cells. FACS analysis showed that a small number of the parthenogenetically derived cells indeed displayed a reduced DNA content (FIG. 8A). Several rounds of FACS purification of this population and subsequent expansion resulted in two independent cell lines derived from two distinct blastocysts, hereafter termed HMSc1 and HMSc2, with a 1n chromosome set in the G1 phase and a 2n chromosome set in the G2 phase of the cell cycle (FIG. 1B, FIG. 8A). Chromosome spreads showed that both cell lines carry a haploid set of 20 chromosomes (FIG. 1C, FIG. 8B). Of note, both cell lines have now been passaged for >50 times without any signs of proliferative crisis. Thus, exploiting activation of meiotic oocytes and parthenogenetic derivation of blastocysts allowed to establish mouse cells with a haploid chromosome set.

### Example 3

### Genome Integrity

To genetically characterize the established haploid cells, their genomes were compared to the genomes of the parental mouse strains C57BL/6 and 129 using deep sequencing and a discriminative coverage analysis was performed. Briefly, the counts of sequencing reads not counting duplicates that map to unique genomic positions in a sliding window of 50 kb (10 kb offset) were assessed, normalized to the total number of reads that mapped to the genome in each library. As expected, it was found that the strains differed at many positions (documentation in the web atwww.starklab.org/data/elling_CSC_{—}2011/). Therefore deep sequencing reads from genomic DNA of both the haploid cell lines to both parental strains individually were compared and in a combined fashion in which focus was on deviations with respect to both parental strain (FIGS. 1D, E). In total, it was found that HMSc1 differed from the parental strains by more than 2 fold (multiple-testing corrected p-value≦10⁻³) in 1553 overlapping windows corresponding to 219 non-overlapping regions, HMSc2 had 568 windows (113 regions) with increased and 61 windows (10 regions) with decreased read-counts (2-fold; multiple-testing corrected p-value≦10⁻³). Of note, 1155 windows with increased and 99 windows with decreased read-counts overlapped between HMSc1 and HMSc2, while only 397 and 4 were distinct, respectively. The inventors do not know the origin of this observation but these shared copy number variations (CNVs) might stem from biases of the sequencing procedure, either during DNA preparation from different cell-types (ES cells vs. adult kidney) or constitute a batch effect during sequencing. All primary sequencing data are available at www.ncbi.nlm.nih.gov/geo/ and at www.starklab.org/data/elling_CSC_{—}2011/ formatted for uploading to the UCSC genome browser (genome.ucsc.edu/). Thus, taken together, the haploid cell lines display a limited number of defined CNVs compared to the parent strains, suggesting that they likely harbor small duplications and deletions similar to previous reports using established ES cell (Baker et al., 2007) or iPS cell lines (Hussein et al., 2011).

To confirm that both haploid cell lines are indeed distinct given the similarity in copy number analysis, the fact that they were derived from oocytes of C57BL/6×129 intercrosses was used, such that meiotic recombination should result in distinct haplotype structures. The deep sequencing reads to SNPs obtained from the Sanger Institute small nucleotide polymorphisms (SNP) release were compared that differed between both mouse strains. Our sequences covered 1.5 million (HMSc2) and 1.7 million (HMSc1) distinct SNPs and allowed a unique assignment of the corresponding genomic regions to one of both parents. Comparing the SNPs present in HMSc1 versus HMSc2 cells indeed confirmed that two independent haploid clones were derived (FIG. 8C). The data are available for download at www.ncbi.nlm.nih.gov/geo/ and www.starklab.org/data/elling_CSC_{—}2011/. In summary, the CNV analysis together with FACS and chromosome spreads show that both cell lines are derived independently and exclude that HMSc1 and HMSc2 have substantial parts of the genome or individual chromosomes deleted or duplicated.

### Example 4

### Expression of Prototypic Stem Cell Markers

It was next tested whether the haploid cell lines express prototypic embryonic stem cell markers, i.e. whether the haploid cells are indeed stem cells. Both parthenogenetically derived haploid HMSc1 and HMSc2 lines exhibited typical morphologies of ES cells and stained positive for the ES cell marker alkaline phosphatase (FIG. 2A). Immunolabelling to detect Oct4, Nanog, and Sox2 (FIG. 2B) confirmed that HMSc1 and HMSc2 cells express prototypic ES cell markers. Transcriptome analysis showed that the expression profile for both haploid cell lines closely resembled that of the diploid ES cell line IB10/C (FIG. 9A). To focus on genes with the highest discriminatory value, a set of 100 genes was analysed for maximum difference in gene expression in either direction between mouse embryonic fibroblasts (MEFs) and diploid ES cells, amongst them Nanog, Oct4 and Klf4 (Chambers et al., 2003). Analysis of this gene set showed that both HMSc1 and HMSc2 cell lines exhibit an expression signature that closely resembles that of the bona fide diploid ES cell line IB10/C (FIG. 9B). Quantitative PCR analyses confirmed that both HMSc1 and HMSc2 cells express prototypic ES cell markers (FIG. 2C) (Elling et al., 2006; Takahashi and Yamanaka, 2006). Taken together, global transcriptome profiling and expression analysis of prototypic stem cell markers confirmed the stem cell nature of both haploid cell lines.

### Example 5

### Differentiation Potential of Haploid Stem Cells in Vitro

To test the differentiation potential of our haploid ES cells, we first assayed for embryoid body (EB) formation. Both haploid cell lines readily underwent EB formation and EB cells expressed the endodermal marker Gata4 (FIG. 2D). Differentiation was further confirmed using real time PCR. The prototypic ES cell markers Nanog, Rex1, Oct4, Sox2, Klf4, Sall4, and Kfl2 (Chambers et al., 2003; Takahashi and Yamanaka, 2006) were down-regulated in EBs while mRNA expression of the lineage commitment markers Hand1 (mesoderm, trophectoderm), Nkx2-5 and Brachyury (both mesoderm), Nestin (neural), Gata4, Gata6, Foxa2 (all early endoderm), Sox17 (endoderm and mesoderm), Cxcr4R (endoderm), and Keratin18 (ectoderm) was upregulated relative to the parental haploid ES cells (FIG. 2E, FIG. 9C). These results indicate that the haploid ES cells are capable of differentiation into several lineages of all three germ-layers.

### Example 6

### In Vivo Differentiation Potential

To evaluate the ability of the established haploid ES cell lines to contribute to adult mice, cells from the Agouti⁺ ES line HMSc2 were injected into ED 3.5 blastocysts. To assure competitive growth and thus efficient contribution, diploid cells derived from haploid HMSc2 were used. Coat colour chimerism was observed in 6 animals out of 25 mice born (FIG. 3A). To analyse contribution of the entirely maternal derived cells to various organs as previously reported for parthenogenotes (Thomson and Solter, 1988), a distinguishing PCR was performed and detected HMSc2 derived cells in multiple tissues (FIG. 10A). To test the intrinsic differentiation potential of our haploid stem cells, in vivo teratoma assays were performed. Similar to diploid ES cell controls, injection of both HMSc1 and HMSc2 cells always resulted in the formation of teratomas within 4-8 weeks.

In teratomas derived from both haploid stem cell lines mesoderm derived muscle cells, endoderm derived alcian blue positive epithelial tissues that produce mucin, neuroectoderm derived Tuj1⁺neurons, as well as ectoderm-derived Cytokeratin 5⁺epithelial tissues were observed (FIG. 3B). In addition, bona fide cartilage tissue, fat, keratinized multilayered epithelium, pigmented epithelium, sebaceous sweat glands, glandular and neuronal tubules, or ciliated respiratory epithelium were observed (FIG. 10B-I). These data show that haploid stem cell derived cells have the potential to contribute to chimeric mice and that they can differentiate in vivo into cells of all three germ layers.

### Example 7

### The Ability of Stable Growth and Differentiation is Intrinsic to Haploid Stem Cells

To assess whether the haploid stem cells have the intrinsic ability for stable growth, several individual cell clones were established by plating single haploid cells directly after FACS purification. These subclones were established in feeder free conditions and derived from both HMSc1 and HMSc2 parental lines that were previously cultured for more than 30 passages. All derived subclones expressed the stem cell markers Oct4 and Sox2 (FIG. 4A, FIG. 11) and formed EBs that contained Gata4⁺endodermal cells and Tuj1⁺neurons (FIG. 4A, FIG. 12). The haploid subclone HMSc2-27 was chosen for further studies based on its growth rates and numbers of stable haploid cells (FIGS. 13A,B).

Typical stem cell morphologies, protein expression of Oct4, Nanog, and Sox2, and a haploid set of chromosomes were confirmed for the HMSc2-27 subclone (FIG. 13C-F). The growth rates of HMSc2-27 cells at different haploid:diploid seeding ratios were comparable to that of purely diploid HMSc2-27 cells (FIG. 4B). Of note, these growth rates are comparable to that of previously established ES cell lines. Kinetic studies on diploid versus haploid cell ratios in cultures of HMSc2-27 cells showed that a large fraction of these cells maintains haploidy for a period of 7 passages (FIG. 4C). Differentiation of HMSc2-27 stem cells into EBs followed by lineage specific differentiation protocols showed that these cells have the capability to form Gata4⁺endoderm, Tuj1⁺neuronal lineage (FIG. 4A), and mesodermal “beating” myoblasts (FIG. 4D, for synchronous contractions see Suppl. Movie 1 and 2). Moreover, in in vivo teratoma assays HMSc2-27 cells can differentiate into cells of all germ layers (not shown). To confirm the subcloning experiment, i.e. to make sure that cloning from a single haploid stem cell indeed works, GFP positive subclones were generated. All cells from the established subclones expressed GFP, irrespective if they were at a stage of haploidy or diploidy (FIG. 4E). Since diploid cells cannot become haploid, these experiments confirm that all cells of such clones must have been derived from a single haploid cell.

The HMSc2-27 subclone allowed us to examine the differentiation potential of these cells at a haploid versus diploid state. Haploid HMSc2-27 stem cells were indeed able to down-regulate Oct4 and form Tuj1⁺neurons and Gata4⁺endodermal cells in EB cultures (FIG. 4F). It was next attempted to differentiate haploid HMSc27 cells directly (without formation of EBs) into neural and astrocyte lineages (Pollard et al., 2006). HMSc2-27 cells were able to differentiate into neural stem cells (NSCs) as defined by Nestin expression. Moreover, upon further differentiation GFAP⁺astrocytes and Tuj1⁺neurons were observed (FIG. 5A). It was next tested whether differentiation and a state of haploidy are mutual exclusive, i.e. whether haploid cells need to become diploid before they differentiate. We therefore gated for the lineage markers Oct4 and Nestin in embryonic stem cells, neural stem cells, and differentiated neural stem cell cultures and assayed cells for DNA content. Whereas nearly all Oct4⁺cells remained haploid under all culture conditions, haploid as well as diploid Nestin⁺neural progenitor cells were observed. However, when such neural progenitors were further differentiated towards astrocytes and neurons, all Nestin positive cells became diploid within 4 days (FIG. 5B).

The differentiation capacity of haploid cells was further assessed using high content screening analysis of Oct4 expression (Walker et al., 2007), DAPI intensity and nuclear area, enabling automated determination of the differentiation state of haploid and diploid HMSc2-27 cells (FIG. 14). Stem cells cultured in presence of LIF for 72 h maintained high levels of Oct4 as depicted by mean Oct4 expression intensity (FIG. 5C, left panel). Distribution analysis showed that HMSc2-27 cells that became diploid expressed a slightly higher level of Oct4 than haploid cells, consistent with the larger nucleus. Upon LIF withdrawal, Oct4 expression substantially decreased in haploid and diploid HMSc2-27 cells (FIG. 5C, middle panel). Moreover, differentiation in response to 0.5 μM retinoic acid dramatically reduced Oct4 expression in haploid and diploid HMSc2-27 cells to background levels (FIG. 5C, right panel), similar to results obtained using 0.1 μM retinoic acid, indicating efficient differentiation. These data show that haploid stem cells can differentiate at similar kinetics as diploid ES cells and, importantly, that haploid stem cells can maintain haploidy even upon initiation of differentiation.

### Example 8

### Retroviral Mutagenesis

The idea to establish haploid stem cells was to create a tool for forward and reverse genetics at the genomic scale. To demonstrate the power of mutagenesis in haploid mouse stem cells, 5×10⁸ cells of a freshly FACS purified haploid culture of HMSc2-27 were infected with a previously reported retrovirus containing a reversible gene trap (Schnutgen et al., 2008). This vector also contains removable Oct4 binding sites (Schnutgen et al., 2008) which allow for insertions into genes that show minimal or none detectable expression in stem cells. After infection, 7.5×10⁶ independent genomic insertions were generated as estimated from colony formation assays.

Stem cell colonies were then pooled and 10 μg of genomic DNA corresponding to 3 million cells were analysed to map the viral insertion sites by inverse PCR and deep sequencing. We could unambiguously identify 176, 178 insertions. About half of the insertions were mapped to intergenic regions and ˜51% of insertions occurred in promoter regions and intragenic regions encompassing 8203 different genes (5′ and 3′ UTR, 1^{st} intronic, other intronic, and coding regions) (FIG. 6A). Among the intragenic insertions, approximately half (53%) were in sense, half in anti-sense direction. Of note, frequent insertions into the 1^{st} intron were observed that most likely will result in complete disruption of gene expression/function. To analyse gene trap efficacy genes were divided into 10 bins based on their expression levels in HMSc2 cells (0-10% equals lowest expression, 90-100% equals highest expressed genes). As expected, more highly expressed genes were more often hit (up to 67%). Importantly, due to the engineered Oct4 binding sites (Schnutgen et al., 2008) we were able to obtain frequent (31%) insertions into genes that show minimal or none detectable expression in stem cells (FIG. 6B). We next analysed the numbers of genes that are trapped by all 176, 178 insertions or fractions of the total insertions (FIG. 6B; all insertions are set to 100% at the X-axis). This analysis shows that mutagenesis has not reached saturation, indicating that higher numbers of insertions will increase the numbers of targeted genes. Considering that the used library consists of 40× more (7.5×10⁶) independent integrations, the mutagenesis protocol has, in principle, the power to disrupt most genes.

### Example 9

### Rapid and Near-Genome Wide Screens Using Repairable Mutagenesis Vectors

The following approaches for insertional mutagenesis were used: Vectors sharing a transcriptional terminating cassette, i.e. a splice acceptor (SA) “catching” a gene transcript if inserted into an intron of a defined gene followed by a polyA signal leading to a premature truncation of the nascent mRNA (FIG. 15A). Integrated gene traps were selected using an antibiotic resistance cassette transcribed from the promoter of the trapped gene. We use such a cassette in a retroviral backbone (FIG. 15B) and cloned it onto in a sleeping beauty transposon backbone (FIG. 15C). This method requires transcription of the locus and is therefore limited to genes transcribed in the respective cell type. Gene trapping has been recently improved by the addition of an embryo stem-like cell-specific enhancer element (FIG. 15D) (Schnuetgen et al. 2008). Thereby, genes weakly expressed in embryo stem-like cell are being activated to levels that allow for sufficient selection. PolyA trapping cassettes insert with their own promoter (light blue arrow in FIG. 15E), but the mRNA remains unstable due to missing polyA signals unless inserted upstream of a genomic polyadenylation signal and thus within a transcript. Therefore, this system does not require any transcription of the targeted gene in embryo stem-like cells.

For delivery of the gene trap cassettes we used retroviral and lentiviral systems as well as several transposable elements, i.e. sleeping beauty, Tol2, and piggyBac (FIG. 15B-E). Delivery systems, virus titers, or numbers of haploid stem cells being used can be optimized for each system in careful titration experiments. The reason for testing all these different mutagenesis strategies is, that all of these systems have their hotspots of integration. Importantly, all our vectors contain the double flex system for inversion of the cassette. This is accomplished by an array of incompatible loxP/lox5171 and FRT/F3 sites (FIG. 15F). This revertible system establishes causality of the identified genes since we can compare mutated and “repaired” clones side-by-side and even revert the insertions twice depending on the question we want to ask. Moreover, we can generate loss of function mutants from antisense insertions and thereby study the function of otherwise lethal house-keeping genes.

Most delivery systems for gene trap cassettes display a bias towards integration sites. However, although these delivery strategies are widely used, surprisingly no systematic comparative study of all methods using large datasets has ever been undertaken. We therefore used our different lentiviral, retroviral, and transposon systems to perform large scale mutagenesis screens in our haploid stem cells. It then is possible to map the tens of millions of independent mutations generated in to the genome. This, for the first time ever, allowed us to systematically and side-by-side compare the insertion behaviour of our mutagenesis vectors. From such datasets we were able to derive multiple parameters such as: 1. Comparing mutagenesis saturation behaviour of each system as well as combinations thereof to set-up the optimal mutagenesis systems for our loss-of-function genetics. 2. By independently analyzing biological replicates of the identical vectors we were able to assess the probability of independent integrations at the identical genomic site. 3. Comparing the probability of each system to hit each gene identifies common hot spots, common cold spots, as well as differentially targeted loci in the genome. 4. Analysis of gene sets showing similar targeting behaviour (e.g. hot spot) allows to correlate common hot/cold spots to methylation status, DNAse accessibility, or the transcriptional state of the cells.

### Example 10

### Haploid Murine Stem Cells as a Tool for High Throughput Reverse Genetics

Using the retroviral mutagenesis set-up we next picked individual clones, identified the insertion sites of about 1000 cell lines, and selected 10 clones with sense or antisense insertions for further analysis. PCR analysis with site specific primers confirmed that our sequencing approach identified the correct target sites in all 10 cases (FIG. 6C). Most importantly these data also show that all 10 clones carry homozygous insertions (FIG. 6D), indicating that mutagenesis has occurred in haploid cells and that this approach is indeed feasible for recessive genetics.

Two clones carrying insertions in the genes encoding the retinoic acid receptor gamma (Rarg) and Drosha were functionally validated using parental wild type (Wt) HMSc2-27 cells, or stem cells clones that carry the retroviral vector in antisense orientation. By transient Cre expression we then converted the alleles to sense integrations in which the splice acceptor disrupts gene expression; this approach allows for immediate confirmation of the candidate gene and excludes potential background mutations. Indeed, sense integrations of the splice acceptor results in a near complete absence of Rarg mRNA expression (FIG. 6E). Functionally, whereas stem cells carrying the wild type (Wt) allele or the splice acceptors in antisense orientation undergo rapid differentiation and cell death upon retinoic acid treatment, disruption of Rarg expression renders stem cells insensitive to such retinoic acid effect (FIG. 6F).

The RNase III Drosha catalyzes the conversion of pri-miRNA transcripts into pre-miRNA stem-loop precursors in the nucleus (Lee et al., 2003). Due to this pivotal role in the initial step of miRNA processing, homozygous Drosha inactivation is predicted to severely impair miRNA biogenesis. While a conditional Drosha knock-out mouse has been published previously (Chong et al., 2008), no viable Drosha knock-out cell line has been reported yet. We were indeed able to generate a Drosha mutant stem cell clone following Cre mediated inversion of the splice acceptor (FIG. 6G). As reported for ES cells with mutations in the pasha orthologue Dgcr8 (Wang et al., 2007), which together with Drosha is part of a protein complex called the Microprocessor complex, drosha mutant stem cell cannot form cystic embryoid bodies (FIG. 6H). To evaluate primary miRNA processing in ES cells harboring the disrupted Drosha allele, we monitored the effects of a potent miR30-based shRNA (shmiR.Luc1309) on expression of a transcript harboring a Luc1309 specific shRNA target site (target) in the 3′UTR of a sequence encoding the Venus reporter protein. While expression of shmiR.Luc1309 strongly suppressed Venus expression in normal ES cells, our Drosha deficient ES cell clone did not show shRNA-mediated reporter suppression (FIG. 6I), indicating a dysfunctional miRNA pathway. These data show that haploid stem cells can be indeed efficiently used for reverse genetics creating reversible and homozygous mutations.

Our analysis of integration behavior of retroviral gene trap vectors showed that about 50% of mapped integrations are in anti-sense to a given gene and consequently non-disruptive. As a result, any integration into an essential gene will be recovered in its anti-sense orientation as the sense orientation results in death and its depletion from the pool of surviving cells after retroviral gene trap mutagenesis. As an example, using our revertable mutagenesis strategy we were the first to describe a viable homozygous knockout stem cell line for Drosha, an enzyme that plays a pivotal role in the initial step of miRNA processing. By transient Cre expression we then converted the antisense to a sense insertion in which the splice acceptor disrupts gene expression. Indeed, sense, but not antisense, integration resulted in a near complete absence of Drosha mRNA expression, and functionally, Drosha mutant stem cells could not form cystic embryoid bodies (FIG. 16).

### Example 11

### A Genome Wide Screen for Genes Involved in Ricin Toxicity

Finally, we set out to perform a recessive forward genetic screen at the genome level using the haploid stem cell system. The naturally occurring ricin toxin from the castor oil plant Ricinus communis is highly poisonous. At the molecular level, ricin binds to N-acetyl galactosamine or beta-1,4-linked galactose residues and mannose receptors on the cell surface and ricin molecules are thought to follow retrograde transport via the Golgi apparatus to enter the lumen of the endoplasmic reticulum (ER) where they escape into the cytosol to inactivate ribosomes. Since we found that ricin is highly toxic to mouse ES cells, we used the mutagenized haploid HMSc2-27 cell library and challenged the cells with a lethal dose of ricin. Whereas ricin killed all control stem cells, we observed growth of multiple stem cell colonies from the mutagenized haploid HMSc2-27 cells (FIG. 7A). These clones where then pooled and deep sequenced to determine the integration sites. As expected from previous studies, multiple enzymes involved in sugar metabolism were found, i.e. beta-1,4-galactosyltransferase 1 (B4galt1), N-acetyllactosaminide beta-1,6-N-acetylglucosaminyl-transferase (Gcnt2), polypeptide N-acetylgalactosaminyltransferase2 (Galnt2), glycoprotein galactosyltransferase alpha 1,3 (Ggta1), and polypeptide N-acetylgalactosaminyltransferase3 (B4galnt3) (FIGS. 7B, C). Multiple disruptive mutations in the alpha(1,3)-fucosyltransferase Fut9 were also obtained and the GDP-fucose transporter) Slc35c1 was hit (FIGS. 7B, C). The fucosylation pathway has until now never been associated with ricin toxicity.

Intriguingly, 49 different integrations in the GPCR Gpr107 (LUSTR1) were observed (FIG. 7B), suggesting that Gpr107 is a key molecule involved in ricin toxicity. Knockdown of Gpr107 expression in MHSc2-27 cells using shRNA technology in HMSc2 confirmed the central role of this GPCR in ricin toxicity. The key role of Gpr107 in ricin-induced cell death was further confirmed in a different cell type, i.e. NIH 3T3 cells (FIGS. 7D, E). Of note, our shRNA data are shown as ratios of viable cells recovered from the plates with ricin vs. without ricin treatment (% survival): around 1% of untransfected, control hairpin transfected, or eGFP negative control cells remained still viable after 48 hours of ricin treatment, as compared to their respective non-treated cells; shRNA mediated knockdown of Gpr107 significantly enhanced survival of ricin exposed HMSc2-27 as well as NIH 3T3 cells, but did not fully protect these cells from ricin toxicity. By contrast, genetic ablation of Gpr107 in haploid HMSc2-27 stem cells as performed for the screen resulted in cells that survived ricin application for 3 weeks and were able to expand and form colonies starting from single, mutated cells. Thus, forward genetic screens are feasible and efficient in our haploid stem cells.

Mutation experiments have been repeated with the following resultant resistance gene identification (Table 1):

TABLE 1

| Gene | DI | I | P(intrD) | P(DIvsI) | P(IvsIBG) | P(DIvsDIBG) | LOFScore |
|---|---|---|---|---|---|---|---|
| Mid1 | 129 | 341 | 0.645859 | 0.938816 | 9.42E−276 | 3.49E−100 | 3.27E−100 |
| Gpr107 | 87 | 98 | 2.95E−15 | 1.29E−15 | 8.17E−55 | 4.64E−68 | 6.00E−83 |
| Tcf7l1 | 49 | 60 | 4.03E−08 | 1.19E−10 | 9.86E−22 | 1.49E−30 | 1.77E−40 |
| B4galt1 | 28 | 35 | 5.59E−08 | 1.47E−14 | 2.93E−08 | 2.85E−21 | 4.20E−35 |
| Fut9 | 25 | 30 | 2.46E−05 | 0.0235193 | 1.44E−17 | 2.61E−17 | 6.14E−19 |
| Nmt1 | 17 | 22 | 0.00128841 | 0.000215327 | 1.08E−08 | 1.69E−11 | 3.65E−15 |
| Smad7 | 9 | 18 | 0.03125 | 9.35E−08 | 0.92129 | 7.68E−06 | 7.18E−13 |
| Gm17536 | 17 | 22 | 0.00359869 | 0.00121683 | 2.79E−06 | 1.53E−08 | 1.86E−11 |
| Ggta1 | 13 | 14 | 0.000244141 | 0.000248549 | 0.000788627 | 8.64E−07 | 2.15E−10 |
| 2010012O05Rik | 6 | 7 | 0.0625 | 0.0055929 | 3.24E−06 | 5.97E−08 | 3.34E−10 |
| Nup62cl | 8 | 13 | 0.0898438 | 0.0170441 | 1.17E−06 | 7.27E−07 | 1.24E−08 |
| Vps54 | 10 | 10 | 0.000976563 | 0.000228339 | 0.0257488 | 6.50E−05 | 1.49E−08 |
| Gm13826 | 3 | 3 | 0.125 | 0.0109739 | 0.000138002 | 1.60E−06 | 1.76E−08 |
| Nuak1 | 11 | 15 | 0.0112305 | 0.00118108 | 0.005344 | 2.08E−05 | 2.45E−08 |
| Trim71 | 13 | 24 | 0.0192871 | 0.00064213 | 0.0501043 | 0.00012247 | 7.86E−08 |
| Gm15247 | 8 | 89 | 0.171875 | 0.827595 | 6.01E−78 | 1.59E−07 | 1.32E−07 |
| Mgat2 | 2 | 2 | 1 | 0.00081633 | 0.172688 | 0.0002252 | 1.84E−07 |
| Cetn4 | 2 | 3 | 1 | 0.00038738 | 0.533114 | 0.00050311 | 1.95E−07 |
| Gm8097 | 3 | 3 | 1 | 0.00059843 | 0.296909 | 0.00060909 | 3.64E−07 |
| Rbpj | 9 | 56 | 0.133423 | 0.00266086 | 0.00071083 | 0.00014549 | 3.87E−07 |
| E130311K13Rik | 3 | 3 | 0.25 | 0.0182242 | 0.00119915 | 2.44E−05 | 4.45E−07 |
| Zbtb2 | 10 | 11 | 0.00585938 | 0.028711 | 0.000331245 | 2.09E−05 | 6.01E−07 |
| Gm17642 | 6 | 7 | 0.0625 | 0.0211243 | 0.000989351 | 4.64E−05 | 9.81E−07 |

I number of insertions
DI number of disruptive insertions
p value for intronic directionality (only sense mutations are disruptive in introns)
p value for disruptive mutations over all mutations P(DI vs I)
p value for integrations and disruptive integrations over back-ground (BG)
LOF loss of function score, a p value

By identifying around 3 million independent insertion sites in the mutated batch of cells we can compare the obtained insertions to the insertions prior to the selection. The LOF score multiplies two p values with one another: for relevant genes, disruptive insertions are expected to be enriched both over background probability P(DIvsDIBG) as well as to be enriched over non disruptive insertions in the gene after selection P(DIvsI) the multiplication of those P values estimates the probability for the genes to be true hits.

To verify hits at the single gene level, we isolated hundreds of single cell clones out of the pool of cells and treated these clones with equal amounts of two retroviruses in a 96-well format. One retrovirus was engineered to expresses GFP and the other encodes the red fluorophore mCherry together with Cre recombinase. The infected cells are selected via a Puromycin resistance encoded by both viruses. If cells harbour a gene trap in sense within an intron that is directly related to the resistant phenotype, Cre-recombinase expression renders the insertion non-disruptive and the mCherry-expressing population will lose the selected phenotype: they regain the wild type function of the mutagenized gene, become again sensitive to the toxin and all red cells will be lost in a population (FIGS. 17A, B). If resistance was caused by an unrelated mutation or integration of the gene trap other than intronic, ratios of GFP to mCherry-Cre expressing cells remain unchanged. This method allows for efficient validation of hundreds of mutations in parallel and distinguishes true hits from “background noise”.

To individually confirm the involvement of three of the top hits of the ricin screen, namely Gpr107, Slc35c1, and Fut9, the two latter being involved in fucosylation, we derived mutant haploid stem cell clones directly isolated directly from the screen and verified them with the GFP/ChemyRed-Cre validation system described above (FIG. 17 B, D). Moreover, we performed gene targeting of Grp107 via homologous recombination. This confirmed the results of the initial screen. The haploid chromosomal state allowed the direct derivation of total knock out lines that properly integrated the targeting cassette using simple PCR-based screening for absence of the wild type locus. These Grp107 targeted cells, together with mutant clones for Slc35c1 and Fut9, were used to genetically confirm the essential role of these genes in ricin resistance. In addition, we will overexpress a tagged version of Gpr107 to regain ricin sensitivity in our knock out cells and perform pull-down experiments combined with mass spectrometry to identify interaction partners of Gpr107.

### Gpr107 Pull Down:

To identify potential binding partners or a function of Gpr107, we performed pull down experiments. A C-terminally tagged (mCherry, Ha, myc) version of the protein was overexpressed in the knock out haploid stem cell background. The modified protein allowed functional reconstitution in our cells as shown by regain of sensitivity to ricin toxin. With this expression construct we performed anti-HA pulldown experiments of Gpr107 in whole cell lysates. Through mass spectrometry of the eluted proteins, we identified several potential interaction partners of Gpr107 that are now under further investigation.

TABLE 2
Further ricin resistance gene targets: An HA-tagged
version of Gpr107 was expressed in a knock out embryonic stem
cell background; pulldown experiments were performed to identify
potential binding partners through mass spectrometry analysis.

| | | peptides | peptides | |
|---|---|---|---|---|
| | protein name | control | sample | function |
| GPR107 | G protein coupled | 0 | 141 | |
| | receptor 107 | | | |
| Mtdh | LYRIC | 0 | 46 | Metadherin, |
| | | | | metastatic adhesion |
| | | | | protein, tight junctions |
| Gnai2 | Guanine nucleotide- | 0 | 19 | Guanine nucleotide- |
| | binding protein G(i) | | | binding protein |
| | subunit alpha-2 | | | G(i), α-2 subunit, |
| | | | | heart, cytokines |
| Ppp1r9a | Neurabin-1 | 0 | 13 | Neural tissue-specific |
| | | | | F-actin-binding |
| | | | | protein I, regulatory |
| | | | | subunit 9A |
| | | | | of protein phosphatase |
| | | | | I, actin |
| | | | | cytoskeleton |
| | | | | reorganization |
| Ppp1r9b | Neurabin-2, | 8 | 136 | Actin filament-binding |
| | Spinophilin | | | protein, cell- |
| | | | | cell adhesion sites, |
| | | | | blocks arrestin |
| | | | | at G protein-coupled |
| | | | | receptors |
| Slc2a3 | Solute carrier family | 0 | 5 | GLUT3 |
| | 2, facilitated glucose | | | |
| | transporter member 3 | | | |
| Canx1 | Calnexin | 3 | 7 | Lectin, |
| | | | | Chaperone in ER |
| Rpn1 | Dolichyl-diphos- | 3 | 8 | ER: N-oligosaccharyl |
| | phooligosaccharide-- | | | transferase |
| | protein | | | |
| | glycosyltransferase | | | |
| | SU 1 | | | |

As shown further below, inhibition of genes of table 1 and 2 are not only responsible for ricin resistance, but resistance to ricin-like toxins as well, especially ribosome inhibitory toxins or AB5 toxins, such as pseudomonas exotoxin A.

The naturally occurring ricin toxin from the castor oil plant Ricinus communis is one of the most dangerous poisons and gained notoriety for its potential use in bioterrorism. Since Ricin efficiently kills our haploid mouse stem cells, we performed a forward genetic screen for ricin toxicity. This screen identified the GPCR Gpr107 as a top candidate required for ricin-induced killing. Since no effective anti-toxins are available for treatment of ricin poisoning, molecular inhibition of the genes of FIG. 7, tables 1 and 2, such as Gpr107 are useful candidates to alleviate ricin toxicity in vivo.

### Example 12

### Pseudomonas Exotoxin A Treatment

Equal numbers of Gpr107 wildtype, knock out and reconstituted cells were treated with different concentrations of pseudomonas exotoxin A for 3 days. The viability was assayed afterwards using alamar blue cell viability assay. Gpr107 knock out cells showed increased survival under the toxin as compared to cells expressing the receptor (FIG. 18F).

### Example 13

### Toxin Resistance for Therapies

### Fucosylation Inhibitor:

As fucosylation was shown to be a major pathway in ricin toxicity in our haploid stem cell screen. Fucosylation is of great interest, because this pathway has never been implicated in ricin toxicity. We tested small molecules that serve as substrates but at the same time inhibit fucosylatransferases intracellularly on human cells. HL-60 cells were treated with 2F-Peracetyl-Fucose (compound 2 in Rillahan et al., 2012, see supplement for synthesis information) for 3 days.

### Fucosyltransferase Inhibitor:

Staining for the fucose bearing epitope SSEA-1 and subsequent FACS analysis did indeed show dosage dependent loss of fucosylation in our cells. The pretreated cells were then grown in the presence of both ricin and the inhibitor for 4 days, and their viability and cell number was examined afterwards. Cells that were pretreated with the fucose analog showed markedly increase viability under the toxin stress, confirming the role of fucosylation in ricin toxicity also in human cells. Various molecules and compounds (e.g. 2-deoxy-D-galactose) have been previously shown to specifically inhibit different mammalian fucosyl-transferases FucTIII, V, VI, and VII (Büchsel et al., 1980, Burkart et al., 2000; Lee et al., 2003, Hosoguchi et al., 2010; Rillahan et al., 2012) and can be used to reduce the effects of ricin toxicity.

### Example 14

### Semicloning

Genetic screening in a haploid background allowed the analysis of thousands of genes simultaneously. However, to evaluate the outcome of a screen and further study the function of a set of genes in vivo, animal models serve as a valuable tool and were generated. Recently, different parthenogenetic and androgenetic murine haploid ESC have been shown to contribute to the germline even after genetic manipulation (Li et al. Nature 2012). This raises the possibility of directly using our targeted murine haploid stem cells for in vivo phenotypic studies. Apart from the conventional blastocyst injections for the production of chimeric mice, haploid stem cells can be used to directly fertilize meiose II oocytes by intracytoplasmatic injection (semicloning). This allows for transmission of the genetic material of the donor haploid ESC to the offspring. Appropriate conditions to maintain or regain an optimal epigenetic state of pluripotency of our haploid stem cells could be achieved by chemical inhibition of GSK3 and MEK kinase activity (2i medium). Semicloning allows the use of knockout lines or available stem cell lines to not only identify novel genes using haploid stem cell genetics as described herein, but to also directly “move” these mutant clones to in vivo mouse experiments.

### REFERENCES

Burkart et al. (2000) Bioorganic & Medicinal Chemistry 8, 1937-1946.
Büchsel et al. (1980) Eur J Biochem 111, 445-453.
Bryja et al. (2006) Nat Protoc 1, 2082-2087.
Carette et al. (2009) Science 326, 1231-1235.
Carette et al. (2011) Nat Biotechnol 29, 542-546.
Carette et al. (2011) Nature 477, 340-343.
Cavaleri and Scholer (2003) Cell 113, 551.
Evans et al. (1981) Nature 292: 154-156.
Fellmann et al. (2011) Mol Cell 41, 733-746.
Hartwell et al. (1974) Science 183, 46-51.
Hosoguchi et al. (2010) J. Med. Chem. 53, 5607-5619.
Kaufman et al. (1983) J Embryol Exp Morphol 73, 249-261.
Krotz et al. (2010) J Assist Reprod Genet. 27(12):743-50.
Latham et al. (2002) Biol Reprod 67, 386-392.
Lee et al., (2003) J Am Chem Soc, 125, 9588-9589
Leeb, M., Wutz, A. (2011) Nature 479 (7371): 131-134.
Li et al. (2012) Nature 490(7420):407-11.
Lorthongpanich et al. (2010) Int J Dev Biol. 54(11-12):1631-40.
Martin (1981) Proc. Natl. Acad. Sci. USA 78:7634-7638.
Mitsui et al. (2003) Cell 113, 643.
Nagy et al. (2008) CSH Protoc 2008, pdb prot4706.
Otaegui et al. (1999) Cloning 1, 111-117.
Otto et al. (2001) Science 292, 2441-2443.
Pollard et al. (2006) Methods Enzymol 418, 151-169.
Schnutgen et al. (2008) Nucleic Acids Res 36, e133.
Ready M. et al. (1984) J Biol Chem 259(24): 15252-15256.
Rillahan et al. (2012) Nature Chemical Biology (8): 661-668
Simmons et al. (1985) Anal Biochem 146, 206-210.
Walker et al. (2010) Cell Stem Cell 6, 153-166.
Walker et al. (2007) Cell Stem Cell 1, 71-86.
Wu et al. (2011) Stem Cells 29(5):755-63
Zuber et al. (2010) Nat Biotechnol 29, 79-83.
Zuber et al. (2011) Nature 478, 524-528
Elling et al. (2006) Proc Natl Acad Sci USA 103, 16319-16324.
Takahashi et al. (2006) Cell 126, 663-676.
Chambers et al. (2003) Cell 113, 643-655.
Thomson et al. (1988) Genes Dev 2, 1344-1351.
Thomson et al. (1995) Proc. Natl. Acad. Sci. USA 92:7844-7848.
Chong et al. (2008) J Exp Med 205, 2005-2017.
Wang et al. (2007) Nat Genet. 39, 380-385.
Yan et al. (2000) Nature 403, 723-724.
Yi et al. (2009) Science 326, 430-433.
U.S. Pat. No. 6,235,970
U.S. Pat. No. 7,592,175
WO 2008/33469 A1
WO 2001/32015 A1
WO 2001/30978 A1
WO 2006/56615 A1
WO 2006/56617 A1
WO 2002/88353 A2
WO 2001/29208 A1

### Post-Priority Documents

Elling et al., Cell Stem Cell 9 (6) (2011): 563-574.
Schimenti, Cell Stem Cell 9 (6) (2011): 488-489.
WO 2012/117254 A1

References are incorporated herein by reference. A citation of any reference is not an acknowledgement of prior art.

1-40. (canceled)

41. A method of generating a mammal cell line of haploid cells comprising obtaining a plurality of haploid cells in an aggregated embryonic stage of development, preferably a blastocyst stage, isolating cells of said plurality, expanding isolated cells of said plurality, selecting and isolating one or more single cells with a haploid genome from said expanded cells, thereby obtaining a cell of a cell line of stably haploid cells, wherein the haploid cells are maintained or grown in feeder cell free culture conditions.

42. The method of claim 41 further comprising the steps of obtaining a haploid parthenogenic oozyte or embryonic cell of said mammal, transferring said oozyte or embryonic cell into a pseudopregnant female, allowing said oozyte or embryonic cell to grow to a multicellular stage, optionally culturing cell of said multicellular stage to a blastocyst stage, thereby providing said plurality of haploid cells in an aggregated embryonic stage of development.

43. The method of claim 41 further comprising culturing said cells of the cell culture and isolating haploid cells from said cell culture after up to 50 passages and expanding said isolated haploid cells as new continued cell culture.

44. The method of claim 41 wherein the parental cell is heterozygous or homozygous, preferably heterozygous.

45. A method for generating a pluripotent haploid embryonic stem cell line derived from a parthenogenetic morula or blastocyst, the method comprising:
(a) activating non-fertilized oocytes from a female subject in vitro to induce parthenogenetic development;
(b) culturing said activated oocytes of step (a) to generate morulae and/or blastocysts;
(c) isolating embryonic stem cells from said morulae and/or blastocysts of step (b) and, optionally, transferring said embryonic stem cells to a cell culture medium that inhibits differentiation of said embryonic stem cells;
(d) subjecting the embryonic stem cells of step (c) to FACS analysis and identifying and/or enriching embryonic stem cells displaying a haploid DNA content; and
(e) optionally, repeated FACS purification of the embryonic stem cells displaying a haploid DNA content and expansion of said embryonic stem cells,
thereby generating a haploid embryonic stem cell line,
wherein the cells are maintained or grown in feeder cell free culture conditions.

46. The method of claim 45, wherein said non-fertilized oocytes of step (a) are in vitro matured oocytes.

47. The method of claim 45, wherein the oocytes of step (a) are activated by exposure of the oocytes to ethanol (preferably 5%) or strontium chloride (SrCl₂) (preferably 25 mM).

48. The method of claim 45, wherein steps (d) and/or (e) is/are repeated at least 5 times.

49. The method of claim 41, wherein the female subject is or the cells are of a mammalian, preferably selected from a human, a non-human primate such as macaca (e.g. Macaca fascicularis, Macaca mulatta) or the genus calithrix or great apes (e.g. gorillas, chimpanzees, and orangutans), a mouse, rat, goat, cat, dog, sheep or camel.

50. The method of any of claim 41, wherein the haploid embryonic stem cells of said cell line are characterized by having a 1n chromosome set in the G1 phase and a 2n chromosome set in the G2 phase of the cell cycle.

51. The method of claim 41, wherein at least 60% of the generated embryonic stem cells of said cell line are haploid.

52. The method of any of claim 41, wherein the haploid embryonic stem cells of said cell line are characterized by expression of one or more of the embryonic stem cell markers Alkaline Phosphatase, Oct4, Sox2, Nanog, Klf4, Rex1, Klf2, cMyc Sall4, and/or SSEA-1.

53. The method of claim 41, wherein the haploid embryonic stem cells of said cell line show stable growth for at least 50 passages.

54. The method of claim 41, wherein the haploid embryonic stem cells of said cell line maintain haploidy for at least 7 passages.

55. The method of claim 41, further comprising differentiating the haploid embryonic stem cells of said cell line.

56. The method of claim 41, further comprising genetically modifying the haploid embryonic stem cells of said cell line.

57. The method of claim 41, wherein comprising maintaining the cells by culturing inner cell mass, cells derived from the inner cell mass or trophoblasts or blastocyst outgrowths, or wherein isolating cells of said plurality comprises individualizing cells of the multicellular stage, e.g. the entire blastocyst, preferably by trypsinization of the multicellular stage, preferably blastocyst.

58. The method of claim 41, wherein an individual cell is proliferated and grown into a cell culture (subcloning).

59. The method of claim 41, wherein the cells are maintained or grown in feeder cell free culture conditions after being maintained on a feeder layer.

60. Morula- or blastocyst-derived embryonic stem-like cell line generated by the method of claim 41, which are capable of being maintained or grown in feeder cell free culture conditions.

61. Morula- or blastocyst-derived embryonic stem-like cell line comprising at least 60% haploid embryonic stem cells, which are capable of being maintained or grown in feeder cell free culture conditions.

62. A test system or kit comprising the morula- or blastocyst-derived embryonic stem-cell line of claim 60.

63. Method of claim 41, wherein the blastocyst-derived embryonic stem-cells are derived from the inner cell mass (ICM) of the blastocyst.

64. Method of claim 41, wherein the blastocyst-derived embryonic stem cells are derived from the trophoblast, preferably Oct4 overexpres trophoblast.

65. A cell culture of cells with a haploid genome, which are capable of being maintained or grown in feeder cell free culture conditions, obtainable by a method of claim 41.

66. Method for mutation analysis comprising providing a cell with a haploid genome, preferably a mammal cell, comprising introducing a mutation into a genetic locus of interest in said cell and observing modified activity of the cell related to said genetic locus.

67. Method according to claim 66, wherein the cell is stable in haploid form for at least 10 passages, preferably a cell obtainable or obtained by a method according to any one of claim 1 to 16.

68. A cell of a cell culture according to claim 65, wherein one or more genes of interest have been inactivated.

69. A haploid mammal cell comprising a knock-out of the gene Drosha and/or Rarg, preferably wherein said cell is of a cell culture according to claim 65.

70. Method of generating cells comprising a phenotype of interest, comprising randomly mutating a plurality of cells with a haploid genome, preferably mammal cells, especially preferred cells obtainable or obtained by a method according to claim 41, and selecting cells having the phenotype of interest.

71. Method of claim 70, wherein the phenotype of interest is or cell survival or cell growth when said cells are contacted with a toxin or growth inhibitor.

72. Method of claim 71, where said toxin is ricin.

73. Method of claim 70, wherein the plurality of cells comprises at least 1000 cells.

74. A haploid cell having a resistance to a toxin, preferably ricin, obtainable by a method of claim 70.

75. A method of screening a cell for genetic targets having activity in toxin resistance comprising generating cells with a toxin resistance according to claim 71 further comprising identifying a mutation in said cells having toxin resistance as compared to cells without said random mutation.

76. A method of identifying a therapeutic agent against a toxin, comprising identifying a genetic target according to claim 35 and contacting a therapeutic candidate molecule with said genetic target or the gene product of said target, preferably in an isolated cell, and identifying binding events of the candidate with the genetic target or gene product or modified resistance of the cell to the toxin.

77. The method of claim 76, wherein the toxin is ricin and preferably the genetic target is selected from Gpr107, Fut9, Tcf711, Slc35c1, Fgfr2, Galnt2, Mid1, B4galt1, B4galnt3, Plcd3, Ror2, Samd4b, Gcnt2 or Ggta1.

78. The method of treating an AB5 toxin poisoning, preferably ricin poisoning, in a subject or inducing an AB5 toxin resistance ricin resistance in a subject comprising inhibiting any one of the genes of table 1, 2, preferably Gpr107, Fut9, Tcf711, Slc35c1, Fgfr2, Galnt2, Mid1, B4galt1, B4galnt3, Plcd3, Ror2, Samd4b, Gcnt2 or Ggta1 to said subject, preferably wherein inhibiting is by administration of an inhibitor, especially an antibody specific for any one of the gene products of the genes of table 1 or 2, preferably Gpr107, Fut9, Tcf711, Slc35c1, Fgfr2, Galnt2, Mid1, B4galt1, B4galnt3, Plcd3, Ror2, Samd4b, Gcnt2 or Ggta1, or an inhibitory nucleic acid, especially comprising or encoding a siRNA or shRNA, especially preferred an inhibitory nucleic acid that reduces mRNA of said genes by at least 20%, especially by at least 50%, in a cell.

79. A modified cell with increased resistance to an AB5 toxin, especially ricin, comprising a reduced expression in any one of genes of table 1 or 2, preferably Gpr107, Fut9, Tcf711, Slc35c1, Fgfr2, Galnt2, Mid1, B4galt1, B4galnt3, Plcd3, Ror2, Samd4b, Gcnt2, Ggta1, or combinations thereof, as compared to an unmodified cell without increased resistance to the toxin.

80. The method of treating an AB5 toxin poisoning, preferably ricin or pseudomonas exotoxin A poisoning, comprising administering a fucosylation inhibitor, preferably a small molecule fucosylation inhibitor, especially preferred wherein said inhibitor inhibits Fut9 and/or Slc35c1.


SEQUENCE LISTING (58 sequences)

SEQ ID NO: 1 - DNA, 27 bp, Artificial Sequence
  Synthetic oligonucleotide primer
gaatgtgagcgcacagggtgatgtgcc

SEQ ID NO: 2 - DNA, 25 bp, Artificial Sequence
  Synthetic oligonucleotide primer
cccacagaacacagtcacagggtcc

SEQ ID NO: 3 - DNA, 19 bp, Artificial Sequence
  Synthetic oligonucleotide primer
gcaagaactctcctccatt

SEQ ID NO: 4 - DNA, 18 bp, Artificial Sequence
  Synthetic oligonucleotide primer
atgcgttcaccagatagc

SEQ ID NO: 5 - DNA, 18 bp, Artificial Sequence
  Synthetic oligonucleotide primer
tcactcacatcgccaatc

SEQ ID NO: 6 - DNA, 20 bp, Artificial Sequence
  Synthetic oligonucleotide primer
cctgtagcctcatactcttc

SEQ ID NO: 7 - DNA, 19 bp, Artificial Sequence
  Synthetic oligonucleotide primer
ctcgcagacctacatgaac

SEQ ID NO: 8 - DNA, 18 bp, Artificial Sequence
  Synthetic oligonucleotide primer
ctcggacttgaccacaga

SEQ ID NO: 9 - DNA, 19 bp, Artificial Sequence
  Synthetic oligonucleotide primer
tctctcttcttcggactcc

SEQ ID NO: 10 - DNA, 18 bp, Artificial Sequence
  Synthetic oligonucleotide primer
ctggacgcagtgtcttct

SEQ ID NO: 11 - DNA, 18 bp, Artificial Sequence
  Synthetic oligonucleotide primer
gtacctcgtccgattcca

SEQ ID NO: 12 - DNA, 21 bp, Artificial Sequence
  Synthetic oligonucleotide primer
catcttcttgctcttcttcag

SEQ ID NO: 13 - DNA, 18 bp, Artificial Sequence
  Synthetic oligonucleotide primer
aacttctcgtctgccagt

SEQ ID NO: 14 - DNA, 21 bp, Artificial Sequence
  Synthetic oligonucleotide primer
gagtcatgtagtgtaccttca

SEQ ID NO: 15 - DNA, 18 bp, Artificial Sequence
  Synthetic oligonucleotide primer
ctcagcgagcctatcttg

SEQ ID NO: 16 - DNA, 19 bp, Artificial Sequence
  Synthetic oligonucleotide primer
agaggatgaagtccaacac

SEQ ID NO: 17 - DNA, 19 bp, Artificial Sequence
  Synthetic oligonucleotide primer
gtgagcctgtatgtaatgc

SEQ ID NO: 18 - DNA, 18 bp, Artificial Sequence
  Synthetic oligonucleotide primer
ctgctggcgtcttagatt

SEQ ID NO: 19 - DNA, 18 bp, Artificial Sequence
  Synthetic oligonucleotide primer
ccttcaaggctgaactca

SEQ ID NO: 20 - DNA, 19 bp, Artificial Sequence
  Synthetic oligonucleotide primer
cgccctttaatcctcttct

SEQ ID NO: 21 - DNA, 22 bp, Artificial Sequence
  Synthetic oligonucleotide primer
ctcctacttcctcttcttctaa

SEQ ID NO: 22 - DNA, 20 bp, Artificial Sequence
  Synthetic oligonucleotide primer
cgtcttgacctgaatacttg

SEQ ID NO: 23 - DNA, 18 bp, Artificial Sequence
  Synthetic oligonucleotide primer
gagccgtgaagatggaag

SEQ ID NO: 24 - DNA, 19 bp, Artificial Sequence
  Synthetic oligonucleotide primer
gtgttcatgccattcatcc

SEQ ID NO: 25 - DNA, 17 bp, Artificial Sequence
  Synthetic oligonucleotide primer
gccgatgaacgccttta

SEQ ID NO: 26 - DNA, 18 bp, Artificial Sequence
  Synthetic oligonucleotide primer
caacgccttccaagactt

SEQ ID NO: 27 - DNA, 16 bp, Artificial Sequence
  Synthetic oligonucleotide primer
ttgccgccgatgactt

SEQ ID NO: 28 - DNA, 18 bp, Artificial Sequence
  Synthetic oligonucleotide primer
cagccttgtgatgttggt

SEQ ID NO: 29 - DNA, 18 bp, Artificial Sequence
  Synthetic oligonucleotide primer
ctgcctccaagtgttgtc

SEQ ID NO: 30 - DNA, 20 bp, Artificial Sequence
  Synthetic oligonucleotide primer
gaacaatgcctatgactcac

SEQ ID NO: 31 - DNA, 20 bp, Artificial Sequence
  Synthetic oligonucleotide primer
ccaagatgatccaactcctt

SEQ ID NO: 32 - DNA, 20 bp, Artificial Sequence
  Synthetic oligonucleotide primer
ggtgctgattctgaacaatg

SEQ ID NO: 33 - DNA, 19 bp, Artificial Sequence
  Synthetic oligonucleotide primer
caccatttgagatgctgag

SEQ ID NO: 34 - DNA, 18 bp, Artificial Sequence
  Synthetic oligonucleotide primer
ggcttatagacccgagga

SEQ ID NO: 35 - DNA, 41 bp, Artificial Sequence
  Synthetic oligonucleotide primer
aatgatacggcgaccaccgagatcgccagtcctccgattga

SEQ ID NO: 36 - DNA, 49 bp, Artificial Sequence
  Synthetic oligonucleotide primer
caagcagaagacggcatacgagttcctattccgaagttcctattctcta

SEQ ID NO: 37 - DNA, 31 bp, Artificial Sequence
  Synthetic oligonucleotide primer
tgattgactacccgtcagcgggggtctttca

SEQ ID NO: 38 - DNA, 25 bp, Artificial Sequence
  Synthetic oligonucleotide primer
agtctctcctttgccctgctactgg

SEQ ID NO: 39 - DNA, 25 bp, Artificial Sequence
  Synthetic oligonucleotide primer
cacaggcattgaacagttttgttgg

SEQ ID NO: 40 - DNA, 25 bp, Artificial Sequence
  Synthetic oligonucleotide primer
ttcgagttatagactgtaatgagcc

SEQ ID NO: 41 - DNA, 25 bp, Artificial Sequence
  Synthetic oligonucleotide primer
cctacactctctagcaacggaagcc

SEQ ID NO: 42 - DNA, 26 bp, Artificial Sequence
  Synthetic oligonucleotide primer
gctgttgtcacccttgtgcataagcc

SEQ ID NO: 43 - DNA, 26 bp, Artificial Sequence
  Synthetic oligonucleotide primer
agatgctgggaatggaaccctggtcc

SEQ ID NO: 44 - DNA, 25 bp, Artificial Sequence
  Synthetic oligonucleotide primer
gtagcttagaaactctggccactgg

SEQ ID NO: 45 - DNA, 26 bp, Artificial Sequence
  Synthetic oligonucleotide primer
cagtgaagtctgaatacagagaatgg

SEQ ID NO: 46 - DNA, 24 bp, Artificial Sequence
  Synthetic oligonucleotide primer
gtccatgcaggtttgagtgactcc

SEQ ID NO: 47 - DNA, 26 bp, Artificial Sequence
  Synthetic oligonucleotide primer
gacctccagctacagaggacagagcc

SEQ ID NO: 48 - DNA, 24 bp, Artificial Sequence
  Synthetic oligonucleotide primer
tgtcaagggcaagagctgcgaagg

SEQ ID NO: 49 - DNA, 26 bp, Artificial Sequence
  Synthetic oligonucleotide primer
ccaatgtcaaaccggaagggagaagg

SEQ ID NO: 50 - DNA, 25 bp, Artificial Sequence
  Synthetic oligonucleotide primer
gagttacagatgactgcgagctgcc

SEQ ID NO: 51 - DNA, 25 bp, Artificial Sequence
  Synthetic oligonucleotide primer
gaagcattgagtagctttacctgcc

SEQ ID NO: 52 - DNA, 24 bp, Artificial Sequence
  Synthetic oligonucleotide primer
gtagacctgacttgactggcttgg

SEQ ID NO: 53 - DNA, 26 bp, Artificial Sequence
  Synthetic oligonucleotide primer
gatgctcatcttaccaaacgcatctc

SEQ ID NO: 54 - DNA, 25 bp, Artificial Sequence
  Synthetic oligonucleotide primer
cttcgaccgtctggtcctcaagagg

SEQ ID NO: 55 - DNA, 25 bp, Artificial Sequence
  Synthetic oligonucleotide primer
gaaaccagcctgatctacatagtgg

SEQ ID NO: 56 - DNA, 23 bp, Artificial Sequence
  Synthetic oligonucleotide primer
acttccgacaagattctcagtcc

SEQ ID NO: 57 - DNA, 24 bp, Artificial Sequence
  Synthetic oligonucleotide primer
cgtgacctttgggtgtgtaatgcc

SEQ ID NO: 58 - DNA, 97 bp, Artificial Sequence
  Synthetic oligonucleotide primer
tgctgttgacagtgagcgcaactagcttattcatagccaatagtgaagccacagatgtattggctatgaataagctagttttgcctactgcctcgga